(19) United States
(12) Patent Application Publication
(10) Pub. No.: US 2011/0262442 A1
(43) Pub. Date: Oct. 27, 2011

(54) COMPOSITIONS FOR TREATING CNS DISORDERS

(75) Inventors: Charles P. Hamilton, Ithaca, NY (US); Nathan Dean Jorgensen, New York, NY (US)

(73) Assignee: Adenios, Inc., Ithaca, NY (US)

(21) Appl. No.: 12/941,515

(22) Filed: Nov. 8, 2010

Related U.S. Application Data
(60) Provisional application No. 61/258,815, filed on Nov. 6, 2009.
(60) Provisional application No. 61/383,678, filed on Sep. 16, 2010.

(51) Int. Cl. A61K 39/395; A61P 25/28; A61K 31/721; A61K 38/16; A61K 31/7076; A61K 31/519

(52) U.S. Cl. 424/139.1; 514/46; 514/267; 514/59; 514/17.8

(57) ABSTRACT

The present invention provides combination therapies for treating a disease, disorder, or condition, and methods thereof.

### CROSS-REFERENCE TO RELATED APPLICATIONS

This application claims priority under 35 U.S.C. §119(e) to U.S. Provisional Applications 61/258,815 and 61/383,678, filed on Nov. 6, 2010 and Sep. 16, 2010, respectively, the entirety of each of which is hereby incorporated by reference.

### BACKGROUND OF THE INVENTION

The capillaries that supply blood to the tissues of the brain constitute the blood brain barrier (BBB) (Goldstein et al., “The Blood-Brain Barrier,” Scientific American 255:74-83 (1986); Pardridge, “Receptor-Mediated Peptide Transport Through the Blood-Brain Barrier,” Endocrin. Rev. 7:314-330 (1986)). The endothelial cells which form the brain capillaries are different from those found in other tissues in the body. Brain capillary endothelial cells are joined together by tight intercellular junctions which form a continuous wall against the passive diffusion of molecules from the blood to the brain and other parts of the central nervous system (CNS). These cells are also different in that they have few pinocytic vesicles which in other tissues allow somewhat unselective transport across the capillary wall. Also lacking are continuous gaps or channels running between the cells which would allow unrestricted passage.

The blood-brain barrier functions to ensure that the environment of the brain is constantly controlled. The levels of various substances in the blood, such as hormones, amino acids and ions, undergo frequent small fluctuations which can be brought about by activities such as eating and exercise (Goldstein et al., “The Blood-Brain Barrier,” Scientific American 255:74-83 (1986); Pardridge, “Receptor-Mediated Peptide Transport Through the Blood-Brain Barrier,” Endocrin. Rev. 7:314-330 (1986)). If the brain was not protected by the blood brain barrier from these variations in serum composition, the result could be uncontrolled neural activity.

The isolation of the brain from the bloodstream is not complete. If it were, the brain would be unable to function properly due to a lack of nutrients and an inability to exchange chemicals with the rest of the body. The presence of specific transport systems within the capillary endothelial cells assures that the brain receives, in a controlled manner, all of the compounds required for normal growth and function. In many instances, these transport systems consist of membrane-associated proteins, which selectively bind and transport certain molecules across the barrier membranes. These transporter proteins are known as solute carrier transporters.

Although it is believed that the BBB serves a protective function under normal conditions by protecting the CNS from exposure to potentially toxic compounds, in CNS disease, the BBB may thwart therapeutic efforts by hindering the entry of therapeutic compounds into the CNS. For example, although many bacterial and fungal infections may be readily treated where the site of the infection is outside the CNS, such infections in the CNS are often very dangerous and very difficult to treat due to the inability to deliver effective doses of drugs to the site of the infection. Similarly, the action of the BBB makes treatment of cancer of the brain more difficult than treatment of cancers located outside the CNS. Even where it may be possible to deliver an effective dose of drug into the CNS by administering very large amounts of drug outside of the CNS, the drug levels outside the CNS (such as in the blood) are then often so high as to reach toxic levels deleterious to the kidneys, liver, and other vital organs. Accordingly, there is need in the art for methods to improve the delivery of compounds into the CNS.

### BRIEF DESCRIPTION OF THE DRAWINGS

FIG. 1 shows a graph demonstrating cd73^{-/-} mice are resistant to Experimental Autoimmune Encephalomyelitis (“EAE”). EAE was induced, disease activity was monitored daily, and the mean EAE score was calculated for cd73−/− (open diamonds, n=11) and wild type (cd73+/+) (closed squares, n=13) mice. The results shown are representative of 11 separate experiments.

FIGS. 2A-D show cd73−/− T cells produce elevated levels of IL-1β and IL-17 and mediate EAE susceptibility when transferred to cd73+/+tcrα−/− mice. FIG. 2A shows the CD4 and FoxP3 expression measured on splenocytes from naïve and day 13 post-EAE induced cd73−/− and wild type mice. FIG. 2B shows splenocytes from naïve and day 13 post-MOG immunized wild type mice which were analyzed for CD4 and CD73 cell surface expression by flow cytometry. FIG. 2C shows sorted cells from immunized wild type or cd73−/− mice which were cultured with 1×104 irradiated splenocytes and 0 or 10 μM MOG peptide. Supernatants were taken at 18 hours and run on a cytokine Bio-plex assay. Results represent the fold change in cytokine levels between the 0 and 10 μM MOG peptide groups. Samples were pooled from 4 mice and are representative of one out of three similar experiments. FIG. 2D shows CD4+ T cells from the spleen and lymph nodes from MOG immunized cd73−/− (open diamonds, n=5) or wild type (closed squares, n=5) mice which were adoptively transferred into T cell deficient cd73+/+tcrα−/− mice. EAE was induced and disease progression was monitored daily. Results are representative of two separate experiments.

FIGS. 3A-L show cd73−/− mice which display little or no CNS lymphocyte infiltration following EAE induction; donor cd73−/− T cells infiltrate the CNS of cd73+/+tcrα−/− recipient mice following EAE induction. Frozen tissue sections from day 13 post-EAE induction wild type (FIGS. 3A-C) and cd73−/− (FIGS. 3D-F) mice were labeled with a CD4 antibody. FIG. 3G shows the mean number of CD4+ infiltrating lymphocytes in the brain and spinal cord quantified per field in frozen tissue sections from day 13 post-EAE induction wild type and cd73−/− mice. Eight anatomically similar fields per brain and 4 fields per spinal cord per mouse were analyzed at 10× magnification (n=5 mice/group). Error bars represent the standard error of the mean. FIGS. 3H-L show frozen tissue sections of hippocampus (FIGS. 3H, 3I, and 3K) and cerebellum (FIGS. 3J and 3L) labeled with a CD4 antibody from EAE-induced tcrα−/− mice that received CD4+ cells from wild type (FIGS. 3H-J) or cd73−/− (FIGS. 3K-L) mice at day 12 (FIG. 3K), 18 (FIGS. 3H and 3L), or 22 (FIGS. 3I and 3J) post-EAE induction. Immunoreactivity was detected with HRP anti-rat Ig plus AEC (red) against a hemotoxylin stained nuclear background (blue). Arrows indicate sites of lymphocyte infiltration. Scale bars represent 500 μm.

FIGS. 4A-K show cd73−/− mice which display little or no CNS lymphocyte infiltration following EAE induction; cd73−/− T cells infiltrate the CNS after transfer to cd73+/+tcrα−/− mice and EAE induction. Frozen tissue sections from day 13 post-EAE induction wild type (FIGS. 4A-C) and cd73−/− (FIGS. 4D-F) mice were labeled with a CD45 antibody. Frozen tissue sections of hippocampus (FIGS. 4G, 4H, and 4J) and cerebellum (FIGS. 4I and 4K) labeled with a CD45 antibody from EAE-induced tcrα−/− mice that received CD4+ cells from wild type (FIG. 4G-I) or cd73−/− (FIGS. 4J-K) mice at day 12 (FIG. 4J), day 18 (FIGS. 4G and 4K), or day 22 (FIGS. 4H and 4I) post EAE induction. Immunoreactivity was detected with HRP anti-rat Ig plus AEC (red) against a hemotoxylin stained nuclear background (blue). Arrows indicate sites of lymphocyte infiltration. Scale bars represent 500 mm.

FIGS. 5A-C show myelin specific T cells do not efficiently enter the brain of cd73−/− mice following EAE induction. Vβ11+ T cells from MOG35-55 immunized transgenic 2d2 mice, which express TCRs specific for MOG35-55, were isolated from the spleen and lymph nodes and adoptively transferred into wild type or cd73−/− mice with concomitant EAE induction. At days 1, 3, 8, and 15 post transfer and EAE induction, spleens (FIG. 5A), lymph nodes (FIG. 5B), and brains (FIG. 5C) were removed and cells were harvested. Cells were analyzed for CD45 and Vβ11 expression by flow cytometry. The data represent the relative fold change (RFC) in the percentage of Vβ11+ cells in the CD45+ population for each organ on each given day. Values were normalized to the percentage of cells found in each organ at 1 day post transfer/EAE induction, with 1.0 equaling the baseline value.

FIGS. 6A-D show adoptively transferred CD73+ T cells from wild type mice can confer EAE susceptibility to cd73−/− mice. FIG. 6A shows CD4+ T cells from the spleen and lymph nodes of MOG immunized wild type mice were enriched and adoptively transferred into wild type (closed squares, n=5) or cd73−/− (open diamonds, n=5) mice followed by concomitant EAE induction. Results are shown from one of two independent experiments. FIG. 6B shows T cells from the spleen and lymph nodes of previously immunized wild type and cd73−/− mice were sorted based on CD4 and CD73 expression and adoptively transferred into cd73−/− mice followed by concomitant EAE induction (n=5/each group). Closed squares represent donor cells from wild type mice that express CD73; open squares represent donor cells from wild type mice that lack CD73 expression; open diamonds represent donor cells from cd73−/− mice. FIG. 6C-D show frozen tissue sections of the CNS choroid plexus from naïve wild type (FIG. 6C, left) and cd73−/− (FIG. 6C, right) mice and wild type mice day 12 post-EAE induction (FIG. 6D) were stained with a CD73 (FIG. 6C) or CD45 (FIG. 6D) specific antibody. Immunoreactivity was detected with HRP anti-rat Ig plus AEC (red) against a hemotoxylin stained nuclear background (blue). Brackets indicate CD73 staining. Arrows indicate CD45 lymphocyte staining Scale bars represent 500 μm.

FIGS. 7A-D show adenosine receptor blockade protects mice from EAE development. FIG. 7A shows mean EAE scores where EAE was induced, disease activity was monitored daily, and the mean EAE score was calculated in wild type (squares) and cd73−/− (diamonds) mice given either drinking water (closed shape) alone or drinking water supplemented with 0.6 g/ml of the broad spectrum adenosine receptor antagonist caffeine (open shape). Results are from one experiment (n=5 mice per group). FIG. 7B shows adenosine receptor mRNA expression levels relative to the GAPDH housekeeping gene in the Z310 murine choroid plexus cell line. Samples were run in triplicate; error bars represent the standard error of the mean. FIG. 7C shows results after mice were treated with the A2A adenosine receptor antagonist SCH58261 at 2 mg/kg (1 mg/kg s.c. and 1 mg/kg i.p.) in 45% DMSO (closed squares, n=4 mice/group) or 45% DMSO alone (open squares, n=5 mice/group) 1 day prior to and daily up to day 30 following EAE induction. These results are representative of two experiments. FIG. 7D shows the mean number of CD4+ infiltrating lymphocytes in the brain and spinal cord quantified per field in frozen tissue sections from day 15 post-EAE induction in SCH58261- and DMSO-treated mice are shown. Eight anatomically similar fields per brain and 4 fields per spinal cord per mouse were analyzed at 10× magnification (n=4 mice). Error bars represent the standard error of the mean.

FIG. 8 shows the A2A adenosine receptor antagonist SCH58261 prevents ICAM-1 upregulation on the choroid plexus following EAE induction. Mice were treated with the A2A adenosine receptor antagonist SCH58261 2 mg/kg (1 mg/kg given s.c. and 1 mg/kg given i.p.) in DMSO (n=4 mice/group) or DMSO alone (n=5 mice/group) 1 day prior to and daily up to day 30 following EAE induction. These results are from one experiment. Frozen tissue sections from day 15 post-EAE induction in SCH58261 and DMSO treated mice were examined for ICAM-1 expression at the choroid plexus. WT treated DMSO (left) or SCH58261 (right) and stained for ICAM-1 (red staining, white arrows) and DAPI (blue, nuclei) at 40× magnification. Images are from 4 separate mice.

FIGS. 9A-B demonstrate that CD73−/− mice, which lack extracellular adenosine and thus cannot adequately signal through adenosine receptors, were treated with NECA, resulting in an almost five fold increase in dye migration vs. the PBS control (FIG. 9A). WT mice treated with NECA also show an increase over control mice (FIG. 9B). Pertussis was used as a positive control, as it is known to induce blood brain barrier leakiness in the mouse EAE model.

FIG. 10 shows adenosine receptor expression on the human endothelial cell line hCMEC/D3.

FIG. 11 shows results after hCMEC/D3 cells were seeded onto transwell membranes and allowed to grow to confluencey; 2×106 Jurkat cells were added to the upper chamber with or without NECA (general adenosine receptor [AR] agonist), CCPA (A1AR agonist), CGS 21860 (A2AAR agonist), or DMSO vehicle; and migrated cells were counted after 24 hours.

FIG. 12 shows results after transwell membranes were seeded with Z310 cells and allowed to grow to confluencey; 2×106 Jurkat cells were added to the upper chamber with or with out NECA (n=1, general AR agonist), CCPA (n=1, A1AR agonist), CGS 21860 (n=1, A2AAR agonist), or DMSO vehicle (n=1); and migrated cells were counted after 24 hours.

FIG. 13 shows results after hCMEC/D3 cells were grown to confluencey on 24 well plates; cells were treated with or without various concentrations of NECA (general AR agonist), CCPA (A1AR agonist), CGS 21860 (A2AAR agonist), DMSO vehicle, or Forksolin (induces cAMP); lysis buffer was added after 15 minutes and the cells were frozen at −80C to stop the reaction; and cAMP levels were assayed using a cAMP Screen kit (Applied Biosystems, Foster City, Calif.).

FIG. 14 shows results of female A1 adenosine receptor knockout (A1ARKO, n=5) and wild type (WT, n=5) mice that were immunized with CFA/MOG35-55+PTX on Dec. 2, 2008 and scored daily for 41 days.

FIGS. 15A-B show brains of wild type mice fed caffeine and brains from CD73−/− mice fed caffeine, as measured by FITC-Dextran extravasation through the brain endothelium.

FIG. 16 shows results in graph form of FITC-Dextran extravasation across the blood brain barrier of wild type mice treated with adenosine receptor agonist, NECA, while SCH58261, the adenosine receptor antagonist inhibit FITC-Dextran extravasation.

FIG. 17 shows results of Evans Blue dye extravasation across the blood brain barrier, as measured by a BioTex spectrophotometer at 620 nm, after mice were treated with adenosine receptor agonist NECA.

FIG. 18 shows results in graphical form that demonstrate PEGylated adenosine deaminase (“PEG-ADA”) treatment inhibits the development of EAE in wild-type mice. EAE was induced, disease activity was monitored daily, and mean EAE score was calculated in wild-type mice given either control PBS vehicle alone or 15 units/kg body weight of PEG-ADA i.p. every 4 days. Closed squares represent wild-type mice given PBS vehicle (n=3); open squares represent wild-type mice given PEG-ADA (n=3). These results are from one experiment. These results demonstrate that adenosine deaminase treatment and adenosine receptor blockade protect wild type mice against EAE induction.

FIGS. 19A-B show results in a graph form of dose-dependent increases in 10,000 Da (FIG. 19A) and 70,000 Da (FIG. 19B) dextrans into WT mouse brain 3 h after i.v. administration of NECA or vehicle (DMSO/PBS) as measured by fluorimetry. n=3 mice/treatment group. Inset is splined scatter plot of data points. Statistics indicate significant differences from vehicle, *P≦0.05 by Mann-Whitney. Data are mean±s.e.m. These results demonstrate that i.v.-administered NECA increases BBB permeability to high molecular weight dextrans.

FIGS. 20A-B show results in graphical form of NECA-mediated increase in BBB permeability. FIG. 20A shows extravasation of 10,000 Da FITC-dextran into WT mouse brain when co-administered with NECA or vehicle (DMSO/PBS). Gray bars=vehicle, black bars=NECA. FIG. 20B shows the results of Extravasation of 10,000 Da Texas Red-dextran into WT mouse brain tissue when injected at indicated times after NECA or vehicle administration. Gray bars=vehicle, black bars=NECA. Insets are splined scatter plots with scaled time on the x-axis; diamonds=vehicle, squares=NECA. n=3 mice/treatment group. Statistics indicate significant differences from vehicle (*), P≦0.05 by Mann-Whitney. Data are mean±s.e.m. These results demonstrate that NECA-mediated increase in BBB permeability is temporally discrete and reversible.

FIGS. 21A-F illustrate that increased BBB permeability depends on A1 and A2A adenosine receptors. FIG. 21A shows relative expression of adenosine receptor subtypes on cultured mouse brain endothelial cells (bEnd.3). Levels of 10,000 Da FITC-dextran in WT and A1 (FIG. 21B) and A2A (FIG. 21B) AR knock-out mouse brain 3 h after i.v. administration of NECA or vehicle (DMSO/PBS), as measured by fluorimetry. Gray bars=vehicle, black bars=NECA. Also shown are ose-dependent entry of 10,000 Da FITC-dextran into WT brain tissue 3 h after i.v. co-administration of the specific A2A AR agonist CGS 21860 (FIG. 21D) or the specific A1 AR agonist CCPA (FIG. 21E), as measured by fluorimetry. (FIG. 21F) Levels of 10,000 Da FITC-dextran in WT mouse brain tissue 3 h after i.v. administration of vehicle, NECA, CCPA, CGS 21680 and in combination. n=3 mice/treatment group. Statistics indicate significant differences from vehicle (*) or from NECA (#), P≦0.05 by Mann-Whitney. Data are mean±s.e.m.

FIGS. 22A-D show results in graphical form demonstrating that the A2A agonist Lexiscan increases BBB permeability to 10,000 Da dextrans. FIG. 22A shows results in graphical form that demonstrate Lexiscan administration increases BBB permeability in mice. Data bars before the axis break represent groups that received 3 Lexiscan injections. The bar after the axis break represents a group that received a single Lexiscan injection. n=3-4 mice/treatment group. FIG. 22B shows Lexiscan increases BBB permeability in rats. n=3-4 rats/treatment group. (c) i.p. administered SCH 58261 decreases BBB permeability to 10,000 Da FITC-dextran in mice. FIG. 22D shows the results in graphical form of BBB permeability in rates to FITC-dextran administered simultaneously with 1 mg [Margaret—should this be mg?] of Lexiscan at 5 minutes. Statistics indicate significant differences from vehicle (*) or from 0.01 μg Lexiscan (**), P≦0.05 by Mann-Whitney. Data are mean±s.e.m.

FIG. 23 shows results in graphical form demonstrating that i.v.-administered antibody to β-amyloid antibody crosses BBB and labels β-amyloid plaques in transgenic mouse brains after single dose of NECA. FIG. 23A shows immunofluorescent microscopic images of hippocamppi of transgenic AD (APP/PSEN) and WT mice treated with i.v.-administered antibody to β-amyloid (Covance 6E10) or not and with 0.8 μg i.v. NECA (left panels) or vehicle (right panels). In mice that did not receive 6E10 i.v., 6E10 was used as a primary antibody to control for the presence of plaques. Blue=DAPI, red=Cy5-antibody labeling 6E10-labeled β-amyloid plaque. Scale bar=50 μm. FIG. 23B is a graph showing the quantification of 6E10-labeled amyloid plaques/slice in transgenic mice treated with NECA or vehicle alone.

FIG. 24 is a schematic showing a model of adenosine receptor signaling and modulation of the BBB. (i) Basal conditions favor a tight barrier. (ii) Antagonism of A2A receptor signaling decreases barrier permeability. (iii) Activation of the A1 or A2AAR results in increased BBB permeability. (iv) Activation of both A1 and A2A ARs results in even more permeability than observed after activation of either receptor alone.

FIG. 25 shows images of actin stress fibers after treatment of brain endothelial cells with agents to either agonize A1 (agonized with CCPA) and A2A (agonized with Lexiscan) adenosine receptors. The images show the induction of actin stress fibers upon A1 and A2A agonist treatment (i.e., treatment with CCPA and Lexiscan, respectively) as compared to treatment with vehicle or media alone. These results demonstrate that adenosine receptor signaling results in changes in transendothelial cell resistance and actomyosine stress fiber formation.

### DETAILED DESCRIPTION OF CERTAIN EMBODIMENTS OF THE INVENTION

The barriers to blood entering the central nervous system (“CNS”) are herein collectively referred to as the blood brain barrier (“BBB”). The BBB is a tremendously tight-knit layer of endothelial cells that coats 400 miles of capillaries and blood vessels in the brain (Ransohoff et al., “Three or More Routes for Leukocyte Migration Into the Central Nervous System,” Nature Rev. Immun. 3:569-581 (2003)). The blood-brain barrier (BBB) is comprised of brain endothelial cells, which form the lumen of the brain microvasculature (see Abbott et al., “Structure and Function of the Blood-Brain Barrier,” Neurobiol. Dis. 37:13-25 (2010)). The barrier function is achieved through tight junctions between endothelial cells that regulate the extravasation of molecules and cells into and out of the central nervous system (CNS) (see Abbott et al., “Structure and Function of the Blood-Brain Barrier,” Neurobiol. Dis. 37:13-25 (2010)). The nearly impermeable junctions between BBB cells are formed by the interdigitation of about 20 different types of proteins. Molecules must enter a BBB cell through membrane-embedded protein transporters or by slipping directly through its waxy outer membrane. Once inside, foreign compounds must avoid a high concentration of metabolic enzymes and a variety of promiscuous protein pumps primed to eliminate foreign substances. Having avoided these obstacles, foreign molecules must then pass through the inner membrane of a BBB cell to finally reach the brain. These elaborate defenses allow the BBB to sequester the brain from potential harm, but the BBB also obstructs delivery of neurological drugs to a site of disease in the brain. Researchers in academia and the biotech and pharmaceutical industries are learning to bypass the BBB or allow it to let potential drugs into the brain. They are designing small drugs that can passively diffuse through the BBB or travel on nutrient transporters to get inside the brain. Others are attaching potential therapeutics designed so that the brain will unwittingly engulf them.

The endothelial cells which form the brain capillaries are different from those found in other tissues in the body (Goldstein et al., “The Blood-Brain Barrier,” Scientific American 255:74-83 (1986); Pardridge, “Receptor-Mediated Peptide Transport Through the Blood-Brain Barrier,” Endocrin. Rev. 7:314-330 (1986)). Brain capillary endothelial cells are joined together by tight intercellular junctions which form a continuous wall against the passive diffusion of molecules from the blood to the brain and other parts of the CNS. These cells are also different in that they have few pinocytic vesicles which in other tissues allow somewhat unselective transport across the capillary wall. Also lacking are continuous gaps or channels running between the cells which would allow unrestricted passage.

The blood-brain barrier functions to ensure that the environment of the brain is constantly controlled. The levels of various substances in the blood, such as hormones, amino acids, and ions, undergo frequent small fluctuations which can be brought about by activities such as eating and exercise (Goldstein et al., “The Blood-Brain Barrier,” Scientific American 255:74-83 (1986); Pardridge, “Receptor-Mediated Peptide Transport Through the Blood-Brain Barrier,” Endocrin. Rev. 7:314-330 (1986)). If the brain was not protected by the blood brain barrier from these variations in serum composition, the result could be uncontrolled neural activity.

The isolation of the brain from the bloodstream is not complete. If this were the case, the brain would be unable to function properly due to a lack of nutrients and because of the need to exchange chemicals with the rest of the body. The presence of specific transport systems within the capillary endothelial cells assures that the brain receives, in a controlled manner, all of the compounds required for normal growth and function. In many instances, these transport systems consist of membrane-associated proteins, which selectively bind and transport certain molecules across the barrier membranes. These transporter proteins are known as solute carrier transporters.

Although the BBB serves to restrict the entry of potentially toxic substances into the CNS, it poses a tremendous hurdle to the delivery of therapeutic drugs into the CNS. It has been estimated that more than 98% of small-molecule drugs less than 500 Da in size do not cross the BBB (See Pardridge, “Brain drug targeting: the future of brain drug development,” Cambridge University Press, Cambridge, UK (2001) and Pardridge, “The Blood-Brain Barrier: Bottleneck in Brain Drug Development,” NeuroRx 2:3-14 (2005)). Current approaches aimed at altering the BBB to permit the entry of therapeutics are either too invasive, painful, can result in permanent brain damage or result in loss of drug efficacy (See Broadwell et al., “Morphologic Effect of Dimethyl Sulfoxide on the Blood-Brain Barrier,” Science 217:164-6 (1982); Hanig et al., “Ethanol Enhancement of Blood-Brain Barrier Permeability to Catecholamines in Chicksm,” Eur. J. Pharmacol. 18:79-82 (1972); Rapoport, “Advances in Osmotic Opening of the Blood-Brain Barrier to Enhance CNS Chemotherapy,” Expert Opin. Investig. Drugs 10:1809-18 (2001); Bidros et al., “Novel Drug Delivery Strategies in Neuro-Oncology,” Neurotherapeutics 6: 539-46 (2009); and Hynynen, “MRI-guided Focused Ultrasound Treatments,” Ultrasonics 50:221-9 (2010)). There is a monumental need to modulate the BBB to facilitate the entry of therapeutic drugs into the CNS. Determining how to safely and effectively do this could affect a very broad range of neurological diseases, such as Alzheimer's disease (AD), Parkinson's disease, multiple sclerosis, neurological manifestations of HIV-AIDS, CNS tumors and many more. Promising therapies are available to treat some of these disorders, but their potential cannot be fully realized due to the tremendous impediment posed by the BBB. Accordingly, there is need in the art for methods to improve the delivery of compounds into the CNS.

In addition, patients suffering from edema, brain traumas, stroke and multiple sclerosis exhibit a breakdown of the BBB near the site of primary insults. The level of breakdown can have profound effects on the clinical outcome of these diseases. For instance, the degree of BBB breakdown in patients suffering from multiple sclerosis (“MS”) is correlated to the severity of the disease. It has been shown using Magnetic Resonance Imaging (“MRI”) that, when a person is undergoing an MS “attack,” the blood-brain barrier has broken down in a section of the brain or spinal cord, allowing white blood cells called T lymphocytes to cross over and destroy the myelin.

In certain embodiments, the present invention provides combination therapies for treating central nervous system diseases and/or disorders. In some embodiments, such diseases and/or disorders are localized within the brain, i.e., within the blood brain barrier. Such combination therapies comprise (a) an agent for increasing blood brain barrier permeability in a subject; and (b) a pharmaceutical agent for treating the disease and/or disorder. Such combination therapies comprise an agent which increases adenosine level and/or bioavailability, modulates adenosine receptors, and/or increases CD73 level and/or activity under conditions effective to increase blood brain barrier permeability in the subject.

It will be understood by those of skill in the art that the barrier between the blood and central nervous system is made up of the endothelial cells of the blood capillaries (blood-brain barrier (“BBB”)) and by the epithelial cells of the choroid plexus (“CP”) that separate the blood from the cerebrospinal fluid (“CSF”) of the central nervous system (“CNS”). Together these structures function as the CNS barrier.

In some embodiments, provided compositions and methods are useful for increasing permeability across the choroid plexus. In some embodiments, A₁ agonism increases permeability of the choroid plexus. In other embodiments, A₁ antagonism increases permeability of the choroid plexus. In some embodiments, the present invention provides a method for administering a therapeutic agent across the choroid plexus of a subject comprising administering to the subject (a) the therapeutic agent; and (b) an agent for increasing the permeability of the choroid plexus in a subject. In some embodiments, the agent for increasing the permeability of the choroid plexus is an A₁ antagonist. In other embodiments, the agent for increasing the permeability of the choroid plexus is an A₁ agonist.

In certain embodiments, the present invention provides a composition comprising (a) an agent for increasing blood brain barrier permeability in a subject; and (b) a pharmaceutical agent for treating the disease and/or disorder. In other embodiments, the present invention provides a method for administering to a patient (a) an agent for increasing blood brain barrier permeability in a subject, in combination with (b) a pharmaceutical agent for treating the disease and/or disorder. In still other embodiments, the present invention provides a method for inhibiting a disease or disorder in a biological sample comprising contacting said biological sample with (a) an agent for increasing blood brain barrier permeability in a subject, in combination with (b) a pharmaceutical agent for treating the disease and/or disorder. In still other embodiments, the present invention provides a method for inhibiting a disease or disorder in a patient comprising administering (a) an agent for increasing blood brain barrier permeability in a subject, in combination with (b) a pharmaceutical agent for treating the disease and/or disorder.

In certain embodiments, provided combination therapies are useful in the treatment of metabolic disorders, such as acid lipase disease, Fabry disease or Wernicke-Korsakoff syndrome.

In certain embodiments, provided combinations are useful in the treatment of behavioral disorders, such as ADHD.

In certain embodiments, provided combinations are useful in the treatment of personality disorders, including anxiety disorders, borderline personality disorders, bipolar disorders, depression, eating disorders, obsessive-compulsive disorders, and schizophrenia.

In certain embodiments, provided combinations are useful in the treatment of dementia, including Alzheimer's disease and Lewy Body disease.

In certain embodiments, provided combinations are useful in the treatment of genetic disorders, including Barth syndrome and Tourette's syndrome.

In certain embodiments, provided combinations are useful in the treatment of cancer, including brain and spinal cancers.

In certain embodiments, provided combinations are useful in the treatment of neurodegenerative and/or neuromuscular diseases or disorders, including Canavan disease, Hallervorden-Spatz disease, Huntington's disease, Lewy Body disease, Lou Gehrig's disease, Machado-Joseph disease, Parkinson's disease, and Restless Leg syndrome.

In certain embodiments, provided combinations are useful in the treatment of pain, including neuropathic pain, central pain syndrome, somatic pain, visceral pain, and headache.

In certain embodiments, provided combinations are useful in the treatment of viral infections, including HIV and Dawson disease.

In certain embodiments, provided combinations are useful in the treatment of sleep disorders, including insomnia, narcolepsy, sleep deprivation and Restless Leg syndrome.

In certain embodiments, provided combinations are useful in the treatment of seizure disorders, including epilepsy.

In certain embodiments, the present invention relates to a method for increasing blood brain barrier permeability in a subject. This method involves administering to the subject an agent which activates both of A1 and A2A adenosine receptors.

In certain embodiments, the present invention also relates to a method for increasing blood brain barrier permeability in a subject. This method involves administering to said subject an A1 adenosine receptor agonist and an A2A adenosine receptor agonist.

In certain embodiments, the present invention further relates to a composition. The composition includes an A1 adenosine receptor agonist and an A2A adenosine receptor agonist, and a pharmaceutically acceptable carrier, excipient, or vehicle.

In certain embodiments, the present invention also relates to a method for delivering a macromolecular therapeutic agent to the brain of a subject. This method includes administering to the subject an agent which activates both of A1 and A2A adenosine receptors and the macromolecular therapeutic agent.

In certain embodiments, the present invention also relates to a method for treating a CNS disease, disorder, or condition in a subject. This method involves administering to the subject at least one agent which activates both of A1 and A2A adenosine receptors and a therapeutic agent.

In certain embodiments, the present invention also relates to a method for treating a CNS disease, disorder, or condition in a subject. This method involves administering to the subject an A1 adenosine receptor agonist, an A2A receptor agonist, and a therapeutic agent.

In certain embodiments, the present invention further relates to a method of temporarily increasing the permeability of the blood brain barrier of a subject. The method comprises selecting a subject in need of a temporary increase in permeability of the blood brain barrier, providing an agent which activates either the A1 or the A2A adenosine receptor, and administering to the selected subject either the A1 or the A2A adenosine receptor agonist under conditions effective to temporarily increase the permeability of the blood brain barrier.

In certain embodiments, the present invention also relates to a method for decreasing blood brain barrier permeability in a subject. This method involves administering to said patient an agent which blocks or inhibits A2A signaling.

In certain embodiments, the present invention also relates to a method of remodeling an actin cytoskeleton of a blood brain barrier endothelial cell. This method involves contacting said endothelial cell with an agent which activates both of A1 and A2A adenosine receptors.

Methods and agents of the present invention provide for an improved treatment of subjects with disorders affecting the blood brain barrier. In addition, the present invention provides improved methods of controlling the blood brain barrier to enhance therapeutic treatment of such patients.

### DEFINITIONS

The expression “dosage form” refers to means by which a formulation is stored and/or administered to a subject. For example, the formulation may be stored in a vial or syringe. The formulation may also be stored in a container which protects the formulation from light (e.g., UV light). Alternatively a container or vial which itself is not necessarily protective from light may be stored in a secondary storage container (e.g., an outer box, bag, etc.) which protects the formulation from light.

The terms “effective amount” and “therapeutically effective amount,” as used herein, refer to the amount of a compound or combination that, when administered to an individual, is effective to treat, prevent, delay, or reduce the severity of a condition from which the patient is suffering. In particular, a therapeutically effective amount in accordance with the present invention is an amount sufficient to treat, prevent, delay onset of, or otherwise ameliorate at least one symptom of a central nervous system disease and/or disorder.

As used herein, an “effective amount” of a compound or pharmaceutically acceptable formulation can achieve a desired therapeutic and/or prophylactic effect. In some embodiments, an “effective amount” is at least a minimal amount of a compound, or formulation containing a compound, which is sufficient for treating one or more symptoms of a disease and/or disorder of the brain and/or central nervous system.

The term “pharmaceutically acceptable salts” or “pharmaceutically acceptable salt” refers to salts derived from treating a compound containing a basic nitrogen with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Alternatively, the term “pharmaceutically acceptable salts” or “pharmaceutically acceptable salt” refers to salts derived from treating a compound containing an acidic moiety with an organic or inorganic base.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

The term “patient,” as used herein, refers to a mammal. In certain embodiments, the term “patient” refers to a human.

The terms “suffer” or “suffering” as used herein refers to one or more conditions that a patient has been diagnosed with, or is suspected to have.

The term “subject”, as used herein, means a mammal and includes human and animal subjects, such as domestic animals (e.g., horses, dogs, cats, etc.).

The terms “treat” or “treating,” as used herein, refers to partially or completely alleviating, inhibiting, delaying onset of, reducing the incidence of, ameliorating and/or relieving a disorder or condition, or one or more symptoms of the disorder, disease or condition.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

“Therapeutically active agent” or “active agent” refers to a substance, including a biologically active substance, that is useful for therapy (e.g., human therapy, veterinary therapy), including prophylactic and therapeutic treatment. Therapeutically active agents include organic molecules that are drug compounds, peptides, proteins, carbohydrates, monosaccharides, oligosaccharides, polysaccharides, nucleoprotein, mucoprotein, lipoprotein, synthetic polypeptide or protein, small molecules linked to a protein, glycoprotein, steroid, nucleic acid, DNA, RNA, nucleotide, nucleoside, oligonucleotides, antisense oligonucleotides, lipid, hormone, and vitamin. Therapeutically active agents include any substance used as a medicine for treatment, prevention, delay, reduction or amelioration of a disease, condition, or disorder. Further detailed description of compounds useful as therapeutically active agents is provided below. A therapeutically active agent includes a compound that increases the effect or effectiveness of a second compound, for example, by enhancing potency or reducing adverse effects of a second compound.

The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism will depend upon a variety of factors including the disorder being treated and the severity of the disorder; activity of specific active agent employed; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### 1. Agents Which Increase the Permeability of the Blood Brain Barrier

As described generally above, in certain embodiments, the present invention provides combination therapies comprising (a) an agent for increasing blood brain barrier permeability in a subject; and (b) a pharmaceutical agent for treating the disease and/or disorder. Agents for increasing blood brain barrier permeability in a subject are described in detail herein and in WO 2009/114533 published Sep. 17, 2009, the entirety of which is hereby incorporated by reference.

Without wishing to be bound by any particular theory, it is believed that extracellular adenosine regulates the entry of immune cells into the central nervous system. Accordingly, BBB permeability is mediated by local adenosine concentration and/or activity of adenosine receptors. Extracellular adenosine is generated by enzymatic activity of cell surface molecule CD73. CD73 (ecto-5′-nucleotidase) is a 70-kD glycosyl-phosphatidylinositol-anchored cell surface molecule with ecto-enzymatic activity. It is abundantly expressed on many cell types including subsets of lymphocytes (Yamashita et al., “CD73 Expression and Fyn-Dependent Signaling on Murine Lymphocytes,” Eur. J. Immunol. 28:2981-2990 (1998), which is hereby incorporated by reference in its entirety), endothelial cells (Yamashita et al., “CD73 Expression and Fyn-Dependent Signaling on Murine Lymphocytes,” Eur. J. Immunol. 28:2981-2990 (1998), which is hereby incorporated by reference in its entirety), and epithelial cells (Strohmeier et al., “Surface Expression, Polarization, and Functional Significance of CD73 in Human Intestinal Epithelia,” J. Clin. Invest. 99:2588-2601 (1997), which is hereby incorporated by reference in its entirety). It is further believed to be part of a purine salvage pathway by degrading nucleoside-5′-monophosphates (AMP and IMP) into nucleotides like adenosine and inosine. Thus, increasing adenosine availability increases BBB permeability. Alternatively or additionally, increasing CD73 level or activity produces additional local adenosine, thereby increasing BBB permeability.

In certain embodiments, agents which increase BBB permeability, for use in a provided combination therapy, are those which increase adenosine levels and/or bioavailability (either directly or indirectly), modulate adenosine receptors, and/or increase CD73 levels and/or activity.

In certain embodiments, agents which increase BBB permeability are agents which increase CD73 levels or activity. Such agents are known in the art and include recombinant CD73 protein, cytokines or other factors capable of inducing endothelial CD73 expression, or by a combination of both therapies as described in U.S. Patent Application Publication No. 2006/0198821, which is hereby incorporated by reference in its entirety. More specifically, suitable agents to be used in this invention include cytokines or other factors that directly or indirectly upregulate transcription of the CD73 gene. In some embodiments, a cytokine suitable for use in this invention is an interferon or an interleukin. When a cytokine is an interferon, the interferon may be alpha-, beta-, gamma-, omega-, or any other interferon, including any of the subtypes of the aforementioned interferons. In some embodiments an interferon is an alpha- or beta-interferon. In some embodiments, an interleukin is capable of inducing endothelial CD73 expression. Examples of such interleukins include, but are not limited to, IL-4, IL-10, IL-13 and IL-20.

In one embodiment, the administration of recombinant CD73 protein, a cytokine, or a combination thereof, is combined with administration of adenosine monophosphate (“AMP”) in order to safeguard the source for adenosine to be produced as a result of the elevated CD73 level, obtained by elevated expression or by direct administering of the recombinant CD73 protein.

Exemplary agents which increase CD73 levels or activity are IFN-Beta, CD38, Indomethacin, T3, Dexamethasone, Lovastatin and Carvedilol.

In some embodiments, agents which increase adenosine levels and/or bioavailability are adenylate kinase inhibitors, which prevent the conversion of AMP to adenosine diphosphate (“ADP”) or adenosine triphosphate (“ATP”), thereby promoting the conversion of AMP into adenosine by CD73. In one embodiment, the administration of recombinant CD73 protein, a cytokine or both may be combined with the administration of an adenylate kinase inhibitor to prevent the conversion of adenosine produced by CD73 into ADP and/or ATP. In another embodiment, the administration of recombinant CD73 protein, a cytokine or both may be combined with the administration of AMP and an adenylate kinase inhibitor.

In some embodiments, agents which increase adenosine levels and/or bioavailability are adenosine deaminase inhibitors, which prevent the decomposition of adenosine. In one embodiment, the administration of recombinant CD73 protein, a cytokine, or a combination thereof, is combined with administration of an adenosine deaminase inhibitor. In another embodiment, administration of recombinant CD73 protein, a cytokine, or combination thereof, is combined with administration of both AMP and an adenosine deaminase inhibitor. In yet another embodiment, administration of recombinant CD73 protein, a cytokine, or a combination thereof, is combined with administration of an adenylate kinase inhibitor in combination with AMP and an adenosine deaminase inhibitor.

Exemplary agents which increase adenosine levels and/or bioavailability are Adenosine, Dipyridamole (Persantine), Formycin A, N-ethylcarboxamide-adenosine, (NECA), Triciribine (TCN), Thio-Cl-IB-MECA, Coformycin, Erythro 9-(2-hydroxy-3-nonyl) adenine hydrochloride, 2′-deoxycoformycin, p-Nitrobenzylthionosine, Colchicine, Phenethylalcohol, Papaverine, Nucleosides and related analogs, ICA riboside, AICA ribotide, 1-β-D-ribofuranosyl-1H-1,2,4-triazole-3-carboxamide and Ribavirin monophosphate.

In certain embodiments, agents which modulate adenosine receptors are capable of increasing BBB permeability through their affinity for the four different adenosine receptor subtypes in specific cell types. In some embodiments, agents which either activate the A₂ₐ adenosine receptor or deactivate the A₁ receptor increase the BBB permeability.

In some embodiments, exemplary adenosine receptor A₂ₐ activators are A₂ₐ agonists, which are well known in the art (Press et al., “Therapeutic Potential of Adenosine Receptor Antagonists and Agonists,” Expert Opin. Ther. Patents 17(8): 1-16 (2007), which is hereby incorporated by reference in its entirety). Other A₂ₐ adenosine receptor agonists include those described in U.S. Pat. No. 6,232,297 and in U.S. Published Patent Application Nos. 2003/0186926, 2005/0054605, 2006/0040888, 2006/0040889, 2006/0100169 and 2008/0064653, which are hereby incorporated by reference in their entirety. Such compounds may be synthesized as described in: U.S. Pat. Nos. 5,140,015, 5,278,150, 5,593,975 and 4,956,345; Hutchinson et al., “CGS 21680C, an A2 Selective Adenosine Receptor Agonist with Preferential Hypotensive Activity,” J. Pharmacol. Exp. Ther., 251: 47-55 (1989); Olsson et al, “N6-Substituted N-alkyladenosine-5′-uronamides: Bifunctional Ligands Having Recognition Groups for A1 and A2 Adenosine Receptors,” J. Med. Chem., 29: 1683-1689 (1986); Bridges et al., “N6-[2-(3,5-dimethoxyphenyl)-2-(2-methylphenyl)ethyl]adenosine and its Uronamide Derivatives: Novel Adenosine Agonists With Both High Affinity and High Selectivity for the Adenosine A2 Receptor,” J. Med. Chem. 31: 1282 (1988); Hutchinson et al., J. Med. Chem., 33:1919 (1990); Ukeeda et al., “2-Alkoxyadenosines: Potent and Selective Agonists at the Coronary Artery A2 Adenosine Receptor,” J. Med. Chem. 34: 1334 (1991); Francis et al., “Highly Selective Adenosine A2 Receptor Agonists in a Series of N-alkylated 2-aminoadenosines,” J. Med. Chem. 34: 2570-2579 (1991); Yoneyama et al, “Vasodepressor Mechanisms of 2-(1-octynyl)-adenosine (YT-146), a Selective Adenosine A2 Receptor Agonist, Involve the Opening of Glibenclamide-sensitive K+ Channels,” Eur. J. Pharmacol. 213(2):199-204 (1992); Peet et al., “Conformationally Restrained, Chiral (phenylisopropyl)amino-substituted pyrazolo[3,4-d]pyrimidines and Purines with Selectivity for Adenosine A1 and A2 Receptors,” J. Med. Chem., 35: 3263-3269 (1992); and Cristalli et al. “2-Alkynyl Derivatives of Adenosine and Adenosine-5′-N-ethyluronamide as Selective Agonists at A2 Adenosine Receptors,” J. Med. Chem. 35(13): 2363-2368 (1992), which are hereby incorporated by reference in their entirety. Additional examples of adenosine A2A receptor agonists are disclosed in U.S. Patent Application Publication 2004/0809916, which is hereby incorporated by reference in its entirety. Particularly suitable A2A adenosine receptor agonists include 4-[2-[[6-Amino-9-(N-ethyl-b-D-ribofuranuronamidosyl)-9H-purin-2-yl]amino]ethyl]benzenepropanoic acid (“CGS 21680”) and Lexiscan®. These adenosine A₂ₐ receptor agonists are intended to be illustrative and not limiting.

Exemplary adenosine A₂ receptor agonists, for use in a combination therapy of the present invention, are apadenoson (BMS068645 or ATL146e), binodenoson, NECA (5′-N-ethylcarboxamidoadenosine), CGS-21680 (2-[p-(2-carboxyethyl)phenylethylamino]-5′-N-ethyl-carboxamidoadenosine), ATL313, MRE0094, GW328267, UK371,104, UK432,097, DPMA (N⁶-(2-(3,5-dimethoxyphenyl)-2-(2-methylphenyl)ethyl)adenosine), CVT3146, binodenoson (MRE0470 or WRC0470), and regadenoson.

Suitable A1 adenosine receptor activators are A₁ adenosine receptor agonists. A₁ adenosine receptor agonists are known to those of skill in the art and include, for example, those described in U.S. Patent Application Publication No. 2005/0054605 A₁ to Zablocki et al., which is hereby incorporated by reference in its entirety. Suitable A₁ adenosine receptor agonists also include, for example, 2-chloro-N⁶-cyclopentyladenosine (“CCPA”), 8-cyclopentyl-1,3-dipropylxanthine (“DPCPX”), R-phenylisopropyl-adenosine, N6-Cyclopentyladenosine, N(6)-cyclohexyladenosine, or combinations thereof.

Without wishing to be bound by any particular theory, it is believed that an agent that inhibits adenosine A₁ receptors (i.e., A₁ receptor antagonists), alone or in combination with an agent that activates A₂ₐ receptors (i.e., an A₂ₐ receptor agonist) increase the permeability of the choroid plexus barrier. Thus, according to one embodiment, the present invention provides a method for increasing the permeability of the choroid plexus in a subject comprising administering to the subject an agent that inhibits A₁ receptors and, optionally, an agent that activates A₂ₐ receptors. In a further embodiment, the present invention provides a method for administering a therapeutic agent across the choroid plexus of a subject comprising administering to the subject (a) the therapeutic agent; and (b) and agent for increasing the permeability of the choroid plexus in a subject.

The A₁ receptor is activated at low adenosine concentration (high affinity). Blocking or deactivation of the A₁ adenosine receptor increases BBB permeability. In certain embodiments, adenosine receptor A₁ deactivators are adenosine receptor A₁ antagonists, which are well known in the art (Press et al., “Therapeutic Potential of Adenosine Receptor Antagonists and Agonists,” Expert Opin. Ther. Patents 17(8):1-16 (2007), which is hereby incorporated by reference in its entirety). Exemplary adenosine receptor A₁ antagonists include, but are not limited to, those described in U.S. Patent Application Publication No. 2008/0027082, U.S. Pat. Nos. 5,446,046 and 5,668,139, 6,117,998 and 7,247,639, which are hereby incorporated by reference in their entirety.

Exemplary adenosine A₁ receptor antagonists are caffeine, theophylline, 8-cyclopentyl-1,3-dimethylxanthine, 8-cyclopentyl-1,3-dipropylxanthine (DPCPX), 8-phenyl-1,3-dipropylxanthine, bamifylline, BG-9719, BG-9928, FK-453, FK-838, rolofylline (KW-3902), N-0861, CGS-15943 (9-chloro-2-(2-furanyl)-[1,2,4]-triazolo[1,5-c]-quinazolin-5-amine, and PSB 36 (1-butyl-8-(hexahydro-2,5-methanopentalen-3a-(1H)-yl-3,7-dihydro-3-(3-hydroxypropyl)-1H-purine-2,6-dione).

Suitable A1-selective receptor agonists according to the present invention include 2-chloro-N⁶-cyclopentyladenosine (“CCPA”), N6-Cyclopentyladenosine, N(6)-cyclohexyladenosine, 8-cyclopentyl-1,3-dipropylxanthine (“DPCPX”), R-phenylisopropyl-adenosine, or combinations thereof.

According to one embodiment of the present invention, activating both the A₁ and A_{2A} adenosine receptors is synergistic as compared to the level of BBB permeability when activating either the A₁ adenosine receptor or A_{2A} adenosine receptor alone. In this context, if the effect of activating the two receptors together (at a given concentration) is greater than the sum of the effects when each receptor is activated individually (at the same concentration), then the activation of both the A₁ and the A_{2A} receptors is considered to be synergistic.

According to certain embodiments of the present invention, the activation of both the A₁ and the A_{2A} receptors is additive. In this context, if the effect of activating the two receptors together (at a given concentration) is equivalent to the sum of the effects when each receptor is activated individually (at the same concentration), then the activation of both the A₁ and the A_{2A} receptors together is considered to be additive.

In one embodiment according to the present invention, the increase in BBB permeability lasts up to 18 hours. In further embodiments, the increase in BBB permeability lasts up to about 17 hours, 16 hours, 15 hours, 14 hours, 13 hours, 12 hours, 11 hours, 10 hours, 9 hours, 8 hours, 6 hours, 4 hours, 3 hours, 2 hours, 1 hour, 30 minutes, 15 minutes, 10 minutes, or 5 minutes.

Another aspect of the present invention relates to increasing blood brain barrier permeability in a subject. This method includes administering to the subject an A₁ adenosine receptor agonist and an A_{2A} adenosine receptor agonist.

In one embodiment, the A₁ adenosine receptor agonist and/or the A_{2A} adenosine receptor agonist are selective agonists. As used herein, “selective” means having an activation preference for a specific receptor over other receptors which can be quantified based upon whole cell, tissue, or organism assays which demonstrate receptor activity.

In one embodiment, the A₁ adenosine receptor agonist and the A_{2A} adenosine receptor agonist may be administered simultaneously. In another embodiment according to the present invention, the A₁ adenosine receptor agonist and the A_{2A} adenosine receptor agonist may be administered sequentially.

In certain embodiments, the A₁ adenosine receptor agonist and the A_{2A} adenosine receptor agonist are formulated in a single unit dosage form. Dosage and formulations according to the present invention are described in further detail below.

In one embodiment, this method further includes the administration of a therapeutic agent. The therapeutic agent may be administered together with one or both of the A₁ adenosine receptor agonist and the A_{2A} adenosine receptor agonist, or may be administered following administration of the A₁ adenosine receptor agonist and/or the A_{2A} adenosine receptor agonist. Suitable therapeutic agents are described in further detail below. In certain embodiments, the agonists may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent.

Another aspect of the present invention relates to a composition. The composition includes an A₁ adenosine receptor agonist, an A_{2A} adenosine receptor agonist, and a pharmaceutically acceptable carrier, excipient, or vehicle.

In one embodiment according to this aspect of the present invention, the A₁ adenosine receptor agonist and/or the A_{2A} adenosine receptor agonist are selective agonists.

### 2. Therapeutic Agents

Therapeutic agents for use in a provided combination therapy include those that treat a variety of CNS diseases. Such therapeutic agents are well known in the art and many are common and typically prescribed agents for the relevant disorder. Dosage ranges for such agents are known to one of ordinary skill in the art and are often found in the accompanying prescription information pamphlet (often referred to as the “label”).

Representative therapeutic agents include cholinesterase inhibitors, NMDA antagonists, beta-secretase inhibitors, amyloid precursor protein inhibitors, kinase inhibitors, angiogenesis inhibitors, selective serotonin reuptake inhibitors, MAO inhibitors, norepinephrine reuptake inhibitors, protein kinase C inhibitors, topoisomerase inhibitors, dopamine agonists, LRRK2 inhibitors, COMT inhibitors, dopa carboxylase inhibitors, alpha-synuclein inhibitors, antibiotics, hormones, enzymes and antivirals.

Exemplary therapeutic agents are set forth in Table 1, below.

TABLE 1
Exemplary Therapeutic Agents

| | THERAPEUTIC | | | |
|---|---|---|---|---|
| | MECHANISM/ | | | |
| DISEASE/ | POTENTIAL | | | |
| DISORDER | THERAPY | MEDICATIONS | DOSE | |
| Acid Lipase | Delivery of | | | |
| Disease | lypolytic enzyme | | | |
| ADHD | | Methylphenidate (Ritalin ®) | 5-20 | mg tablets |
| | | Dexmethylphenidate | 2.5-10 | mg tablets |
| | | (Focalin ®) | | |
| | | Amphetamine- | 3.13-18.8 | mg |
| | | Dextroamphetamine | | tablets |
| | | (Adderall ®) | | |
| | | Lisdexamfetamine | 20-70 | mg tablets |
| | | (Vyvanse ®) | | |
| Alzheimer's | Cholinesterase | Donepezil (Aricept ®) | 5-10 | mg tablets |
| Disease | inhibitors | Galantamine (Razadyne ®) | 8-24 | mg tablets |
| | | Rivastigmine (Exelon ®) | 1.5-6 | mg tablets |
| | NMDA antagonist | Memantine (Namenda ®) | 5-10 | mg tablets |
| | Inhibition/ | | | |
| | metabolism of | | | |
| | Amyloid Precursor | | | |
| | Protein (APP) | | | |
| | Regulation of | | | |
| | Presenilin 1 | | | |
| | Regulation of | | | |
| | Presenilin 2 | | | |
| | Regulation of | | | |
| | BACE | | | |
| | Delivery of EPOE | | | |
| Anxiety | | Alprazolam (Xanax ®) | 0.25-2 | mg tablets |
| Disorders | | Clonazepam (Klonopin ®) | 0.5-2 | mg tablets |
| | | Diazepam (Valium ®) | 2-10 | mg tablets |
| | | Escitalopram (Lexapro ®) | 5-20 | mg tablets |
| | | Fluoxetine (Prozac ®) | 10-40 | mg tablets |
| | | Gabapentin (Neurontin ®) | 100-800 | mg tablets |
| | | Hydroxyzine | 10-50 | mg tablets |
| | | Imipramine (Tofranil ®) | 10-50 | mg tablets |
| | | Paroxetine (Paxil ®) | 10-40 | mg tablets |
| | | Phenelzine (Nardil ®) | 15 | mg tablets |
| | | Piperazines | | |
| | | Pregabalin (Lyrica ®) | 25-300 | mg |
| | | | | capsules |
| | | Sertraline (Zoloft ®) | 25-100 | mg tablets |
| | | Tranylcypromine (Parnate ®) | 10 | mg tablets |
| | | Venlafaxine (Effexor ®) | 25-100 | mg tablets |
| Barth | Delivering | | | |
| Syndrome | acyltransferase | | | |
| Bipolar | | Lithium carbonate (Eskalith ®) | 450 | mg tablets |
| Disorder | | Lamotrigine (Lamictal ®) | 25-200 | mg tablets |
| | | Sodium Valproate | 250-500 | mg tablets |
| | | (Depakote ®) | | |
| | | Carbamazepine (Tegretol ®) | 100-400 | mg tablets |
| | | Quetiapine (Seroquel ®) | 25-400 | mg tablets |
| | | Chlorpromazine (Thorazine ®) | 10-200 | mg tablets |
| | | Topiramate (Topomax ®) | 25-200 | mg tablets |
| Brain Cancer | | Interferons | | |
| | | Vesicular Stomatitis Virus | | |
| | | Trastuzumab (Herceptin ®) | 21 | mg/mL solution |
| | | Rituximab (Rituxan ®) | 10 | mg/mL solution |
| | Regulating | | | |
| | hormones | | | |
| | Angiogenesis | Bevacizumab (Avastin ®) | 25 | mg/mL solution |
| | inhibitor | | | |
| | Kinase inhibitor | Imatinib (Gleevec ®) | 100-400 | mg tablets |
| | | Temozolomide (Temodar ®) | 5-250 | mg capsules |
| | | Gefitinib (Iressa ®) | 250 | mg tablets |
| | Alkylating | Cisplatin | 1 | mg/mL solution |
| | antineoplastic agent | Carboplatin (Paraplatin ®) | 10 | mg/mL |
| | | Oxaplatin | | |
| | | Mechlorethamine | 1 | mg/mL solution |
| | | (Mustargen ®) | | |
| | | Cyclophosphamide | 25-50 | mg tablets |
| | | (Cytoxan ®) | | |
| | | Chlorambucil (Leukeran ®) | 2 | mg tablets |
| | | Ifosfamide (Ifex ®) | 1000-3000 | mg/mL |
| | | | | solution |
| | Metabolism | Azathioprine (Azasan ®) | 75-100 | mg tablets |
| | inhibition | Mercaptopurine (Purinethol ®) | 50 | mg tablets |
| | | Vinca alkaloids | | |
| | | Taxanes | | |
| | | Podophyllotoxin | | |
| | Topoisomerase | Irinotecan (Camptosar ®) | 20 | mg/mL solution |
| | Inhibitor | Topotecan (Hycamtin ®) | 1 | mg capsules |
| | | Amsacrine | | |
| | | Etoposide (VePesid ®) | 50 | mg capsules |
| | Antitumor | Dactinomycin (Cosmegen ®) | 0.5 | mg/vial |
| | Antibiotics | Doxorubicin (Adriamycin ®) | 2 | mg/mL solution |
| | | Epirubicin (Ellence ®) | 2 | mg/mL solution |
| | | Bleomycin (Blenoxane ®) | solution | |
| Borderline | | Atypical antipsychotic | | |
| Personality | | medications | | |
| Disorder | | Antipsychotic medications | | |
| | | Olanzapine (Zyprexa ®) | 2.5-20 | mg tablets |
| | | Clozapine (Clozaril ®) | 25-200 | mg tablets |
| | | Quetiapine (Seroquel ®) | 25-400 | mg tablets |
| | | Risperidone (Risperdal ®) | 0.25-4 | mg tablets |
| | | Lithium carbonate (Eskalith ®) | 450 | mg tablets |
| | | Lamotrigine (Lamictal ®) | 25-200 | mg tablets |
| Canavan | Delivering | | | |
| Disease | aspartoacyclase | | | |
| | enzyme | | | |
| Dawson | Measles virus | Antivirals | | |
| Disease | | | | |
| Depression | SSRIs | Escitalopram (Lexapro ®) | 5-20 | mg tablets |
| | | Fluoxetine (Prozac ®) | 10-40 | mg tablets |
| | | Paroxetine (Paxil ®) | 10-40 | mg tablets |
| | | Citalopram (Celexa ®) | 10-40 | mg tablets |
| | | Bupropion (Wellbutrin ®) | 75-100 | mg tablets |
| | | Venlafaxine (Effexor ®) | 25-100 | mg tablets |
| | MAO Inhibition | Selegiline (Eldepryl ®) | 5 | mg capsule |
| | | Rasagiline (Azilect ®) | 0.5-1 | mg tablets |
| | | Protriptyline (Vivactil ®) | 5-10 | mg tablets |
| | | Imipramine (Tofranil ®) | 10-50 | mg tablets |
| | | Clomipramine (Anafranil ®) | 25-75 | mg tablets |
| Eating | | Fluoxetine (Prozac ®) | 10-90 | mg tablets |
| Disorders | | Paroxetine (Paxil ®) | 10-40 | mg tablets |
| Fabry Disease | Delivering alpha- | | | |
| | galactosidase A | | | |
| Hallervorden- | Delivering | | | |
| Spatz Disease | Pantothenate | | | |
| | Kinase 2 | | | |
| Headache | | Acetaminophen/Paracetamol | 120-650 | mg tablets |
| | | Acetylsalicylic acid/Aspirin | 352 | mg tablets |
| | | Diclofenac (Voltaren ®) | 75 | mg tablets |
| | | Ibuprofen | 200 | mg tablets |
| HIV | | Nelfinavir (Viracept ®) | 250-625 | mg tablets |
| Huntington's | | Tetrabenazine (Xenazine ®) | 12.5-25 | mg tablets |
| Disease | | Valproic acid (Stavzor ®, | 125-500 | mg tablets |
| | | Depakene ®) | | or capsules |
| | | SSRIs | | |
| | | Atypical Antipsychotics | | |
| | | Amantadine (Symmetrel ®) | 100 | mg tablets |
| | | Remacemide | | |
| Lewy Body | Inhibition of | Donepezil (Aricept ®) | 5-10 | mg tablets |
| Disease | Cholinesterases | Rivastigmine (Exelon ®) | 1.5-6 | mg tablets |
| | | Galantamine (Razadyne ®) | 8-24 | mg tablets |
| | | Carbodopa-Levodopa | 10/100-25/250 | mg |
| | | (Sinemet ®) | | tablets |
| | | Clonazepam (Klonopin ®) | 0.5-2 | mg tablets |
| | | Methylphenidate (Ritalin ®) | 5-20 | mg tablets |
| | | Modafinil (Provigil ®) | 100-200 | mg tablets |
| | | Riluzole (Rilutek ®) | 50 | mg tablets |
| Lou Gehrig's | Blocking Ion | | | |
| Disease (ALS) | Channels | | | |
| | Inhibiting Protein | Arimoclomol | | |
| | Kinase C | IGF-1 (Increlex ®) | 10 | mg/mL solution |
| | | Minocycline (Minocin ®) | 50-100 | mg |
| | | | | capsules |
| | | KNS-760704 | | |
| Machado- | | Baclofen (Kemstro ®) | 10-20 | mg tablets |
| Joseph Disease | | Levodopa | 10-250 | mg tablets |
| Narcolepsy | Norepinephrine | Atomoxetine (Strattera ®) | 10-100 | mg |
| | Ruptake Inhibitors | | | capsules |
| | | Clomipramine (Anafranil ®) | 25-75 | mg capsules |
| | | Codeine | 15-60 | mg tablets |
| | | Dextroamphetaine (Adderall ®) | 3.13-18.8 | mg |
| | | | | tablets |
| | | Gamma Hydroxybutyrate | | |
| | | Imipramine | 10-50 | mg tablets |
| | | Methamphetamine | 5 | mg tablets |
| | | (Desoxyn ®) | | |
| | | Methylphenidate (Ritalin ®) | 5-20 | mg tablets |
| | | Modafinil (Provigil ®) | 100-200 | mg tablets |
| | | Protriptyline (Vivactil ®) | 5-10 | mg tablets |
| | | Selegiline (Eldepryl ®) | 5 | mg capsules |
| | | Tricyclic Antidepressants | | |
| Obsessive- | | Atypical Antidepressants | | |
| Compulsive | | Benzodiazepines | | |
| Disorder | | Carbamazepine (Tegretol ®) | 100-400 | mg tablets |
| | | Chlorpromazine (Thorazine ®) | 10-200 | mg tablets |
| | | Clomipramine (Anafranil ®) | 25-75 | mg capsules |
| | | Escitalopram (Lexapro ®) | 5-20 | mg tablets |
| | | Fluoxetine (Prozac ®) | 10-90 | mg tablets |
| | | Lamotrigine (Lamictal ®) | 25-200 | mg tablets |
| | | N-Acetylcysteine | 100-200 | mg/mL |
| | | | | solution |
| | | Olanzapine (Zyprexa ®) | 2.5-20 | mg tablets |
| | | Paroxetine (Paxil ®) | 10-40 | mg tablets |
| | | Quetiapine (Seroquel ®) | 25-400 | mg tablets |
| | | Topiramate | 15-200 | mg tablets |
| | | Tricyclic Antidepressants | | |
| | SSRI | | | |
| Pain | | NSAIDs | | |
| | | COX-2 Inhibitors | | |
| | | Morphine (Avinza ®) | 20-120 | mg |
| | | | | capsules |
| | | Codeine | 15-60 | mg tablets |
| | | Hydrocodone | 5-10 | mg tablets |
| | | Diamorphine | | |
| | | Meperidine/Pethidine | 50-100 | mg tablets |
| | | (Demerol ®) | | |
| | | Tramadol (Ultracet ®) | 37.5-300 | mg |
| | | | | tablets |
| | | Buprenorphine (Buprenex ®) | 0.325 | mg/mL |
| | | | | solution |
| | | Amitriptyline (Elavil ®) | 10-150 | mg tablets |
| | | Paracetamol | 120-650 | mg tablets |
| | | Ibuprofen | 200 | mg tablets |
| | | Naproxen | 200 | mg tablets |
| | | Opiates | | |
| Parkinson's | Dopa Carboxylase | Carbidopa (Lodosyn ®) | 25 | mg tablets |
| Disease | Inhibitors | Benserazide | | |
| | | Cardidopa-Levodopa | 10/100-25/250 | mg |
| | | (Sinemet ®, Parcopa ®) | | tablets |
| | | Benserazide/Levodopa | | |
| | | (Madopar ®) | | |
| | COMT Inhibition | Tolcapone (Tasmar ®) | 100-200 | mg tablets |
| | Dopamine Agonist | Bromocriptine (Parlodel ®) | 2.5 mg tablets; 5 mg | |
| | | | capsules | |
| | | Pergolide (Permax ®) | 0.05-1 | mg tablets |
| | | Pramipexole (Mirapex ®) | 0.125-1.5 | mg |
| | | | | tablets |
| | | Ropinirole (Requip ®) | 0.25-5 | mg tablets |
| | | Piribedil | | |
| | | Cabergoline (Dostinex ®) | 0.5 | mg tablets |
| | | Apomorphine (Apokyn ®) | 10 | mg/mL solution |
| | | Lisuride | | |
| | MAO-B Inhibition | Selegine | | |
| | | Rasagiline (Azilect ®) | 0.5-1 | mg tablets |
| | | Amantadine (Symmetrel ®) | 100 | mg tablets |
| | | Benztropine (Cogentin ®) | 1 | mg/mL solution |
| | | Trihexyphenidyl (Artane ®) | 2-5 | mg tablets |
| | | Selegiline/Deprenyl | 1.25-5 | mg tablets |
| | | (Eldepryl ®) | | |
| | | Entacapone (Comtan ®) | 200 | mg tablets |
| | Inhibition of alpha- | | | |
| | synuclein | | | |
| | Inhibition of | | | |
| | LRRK2 | | | |
| | Inhibition of DJ-1 | | | |
| | Delivery of Parkin | | | |
| | Delivery of Pink1 | | | |
| Restless leg | | Ropinirole (Requip ®) | 0.25-5 | mg tablets |
| Syndrome | | Pramipexole (Mirapex ®) | 0.125-1.5 | mg |
| | | | | tablets |
| | | Rotigotine (Neupro ®) | 2-6 | mg/24 h |
| | | Opioids | | |
| | | Benzodiazepines | | |
| Schizophrenia | | Amisulpride (Solian ®) | 50-1200 | mg tablets |
| | | Aripiprazole (Abilify ®) | 2-30 | mg tablets |
| | | Asenapine (Saphris ®) | 5-10 | mg tablets |
| | | Chlorpromazine (Thorazine ®) | 10-200 | mg tablets |
| | | Chlorprothixene | 100-200 | mg tablets |
| | | Clozapine (Clozaril ®) | 25-200 | mg tablets |
| | | Droperidol (Droleptan ®) | 2.5 | mg/mL |
| | | Flupenthixol | | |
| | | Fluphenazine (Prolixin ®) | 1-10 | mg tablets |
| | | Haloperidol (Haldol ®) | 0.5-20 | mg injection |
| | | Iloperidone (Fanapt ®) | 1-12 | mg tablets |
| | | Levomepromazine (Nozinan ®) | | |
| | | Mesoridazine | | |
| | | Olanzapine (Zyprexa ®) | 2.5-20 | mg tablets |
| | | Paliperidone (Invega ®) | 1.5-9 | mg tablets |
| | | Periciazine | | |
| | | Perphenazine | 2-16 | mg tablets |
| | | Pimozide (Orap ®) | 1-2 | mg tablets |
| | | Prochlorperazine | 5-10 | mg capsules |
| | | (Compazine ®) | | |
| | | Promazine | 10-200 | mg tablets |
| | | Promethazine (Phenergan ®) | 12.5-50 | mg tablets |
| | | Quetiapine (Seroquel ®) | 25-400 | mg tablets |
| | | Risperidone (Risperdal ®) | 0.25-4 | mg tablets |
| | | Sertindole | | |
| | | Thioridazine (Mellaril ®) | 10-100 | mg tablets |
| | | Thiothixene (Navane ®) | 1-10 | mg capsules |
| | | Trifluoperazine (Stelazine ®) | 1-10 | mg tablets |
| | | Triflupromazine | | |
| | | Ziprasidone (Geodon ®) | 20-80 | mg capsules |
| | | Zotepine | 75-150 | mg tablets |
| | | Zuclopenthixol | 10-40 | mg tablets |
| | mGluR2 Agonism | | | |
| Seizure | | Beclamide | | |
| Disorder | | Brivaracetam | | |
| | | Carbamazepine (Carbatrol ®, | 100-400 | mg tablets |
| | | Tegretol ®) | | |
| | | Clobazam (Frisium ®) | | |
| | | Diastat ® | 2-10 | mg gel |
| | | Ethosuximide(Zarontin ®) | 250 | mg tablets |
| | | Felbamate (Felbatol ®) | 400-600 | mg tablets |
| | | Fosphenytoin (Cerebyx ®) | 50 | mg/mL |
| | | Gabapentin (Neurontin ®) | 100-800 | mg tablets |
| | | Hydantoins | | |
| | | Rufinamide (Inovelon ®, | 200-400 | mg tablets |
| | | Banzel ®) | | |
| | | Lamotrigine (Lamictal ®) | 2-25 | mg tablets |
| | | Levetiracetam (Keppra ®) | 250-1000 | mg |
| | | | | tablets |
| | | Mesuximide (Celontin ®) | 150-300 | mg tablets |
| | | Neurotrin | | |
| | | Nitrazepam | 5-10 | mg tablets |
| | | Phenacemide | | |
| | | Pheneturide | | |
| | | Phenobarbitol (Luminal ®) | 15-100 | mg tablets |
| | | Phenytoin (Dilantin, | 100 | mg capsules |
| | | Phenytek ®) | | |
| | | Pregabalin (Lyrica ®) | 50-225 | mg |
| | | | | capsules |
| | | Primidone (Mysoline ®) | 250 | mg tablets |
| | | Pyrimidinediones | | |
| | | Vigabatrin (Sabril ®) | 500 | mg oral |
| | | | | solution |
| | | Stiripentol (Diacomit ®) | 250-500 | mg tablets |
| | | Temazepam (Restoril ®) | 7.5-30 | mg capsules |
| | | Tiagabine (Gabitril ®) | 2-16 | mg tablets |
| | | Topiramate (Topamax ®) | 15-200 | mg tablets |
| | | Trileptal (Oxcarbazepine ®) | 150-600 | mg tablets |
| | | Valnoctamide | | |
| | | Valproic Acid (Depakene ®) | 125-600 | mg tablets |
| | | Valproamide (Depamide ®) | | |
| | | Lacosamide (Vimpat ®) | 400-600 | mg tablets |
| | | Zonisamide (Zonegran ®) | 25-100 | mg |
| | | | | capsules |
| Tourette's | | Typical Antipsychotics | | |
| Syndrome | | Atypical Psychotics | | |
| | | Fluphenazine (Prolixin ®) | 1-10 | mg tablets |
| | | Haloperidol (Haldol ®) | 0.5-20 | mg injection |
| | | Pimozide (Orap ®) | 1-2 | mg tablets |
| | | Risperidone (Risperdal ®) | 0.25-4 | mg tablets |
| | | Ziprasidone (Geodon ®) | 20-80 | mg capsules |
| Viral Infection | | Tenofovir (Viread ®) | 300 | mg tablets |
| | | Zanamivir (Relenza ®) | 5 | mg powder |
| | | Oseltamivir (Tamiflu ®) | 30-75 | mg capsules |
| | | Valaciclovir (Valtrex ®) | 500-1000 | mg |
| | | | | capsules |
| Wernicke- | Thiamine Delivery | | | |
| Korsakoff | | | | |
| Syndrome | | | | |

In some embodiments, the pharmaceutical agent is a macromolecular therapeutic agent.

### 3. Pharmaceutically Acceptable Compositions

As generally described above, in certain embodiments, the present invention provides a combination therapy comprising administering to a patient suffering from a CNS disease and/or disorder (a) one or more agents for increasing blood brain barrier permeability in a subject; and (b) one or more pharmaceutical agents for treating the disease and/or disorder. In some embodiments, a combination therapy of the present invention is provided in a composition. In certain embodiments, the present invention provides a composition comprising (a) one or more agents for increasing blood brain barrier permeability in a subject; and (b) one or more pharmaceutical agents for treating the disease and/or disorder. In some embodiments, the present invention provides a composition comprising (a) one or more agents for increasing blood brain barrier permeability in a subject; and (b) one or more pharmaceutical agents for treating the disease and/or disorder, and a pharmaceutically acceptable adjuvant, carrier, or vehicle.

The amount of therapeutic agent in a provided composition is such that it is effective to alleviate or lessen the severity of one or more symptoms associated with diseases and/or disorders as described herein. In certain embodiments, a provided composition is formulated for administration to a patient in need thereof. In some embodiments, a provided composition is formulated for oral administration to a patient. In other embodiments, a provided composition is formulated for parenteral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of provided compositions of this invention may be aqueous or oleaginous suspension. Such suspensions may be formulated according to techniques known in the art using suitable dispersing or wetting agents and suspending agents. A sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### 4. Pharmaceutically Acceptable Formulations

Useful carriers for use in inventive pharmaceutical formulations are compatible with the other ingredients in the composition. According to the present invention, agents for increasing blood brain barrier permeability may be administered with therapeutic agents in a single pharmaceutical formulation, or in multiple formulations. Where multiple formulations are employed, each may include both the agent for increasing blood brain barrier permeability and the therapeutic agent, or alternatively, each may include only one.

While it is possible for active agent of a provided combination to be administered as the raw chemical, it is often desirable to present them in the context of one or more pharmaceutical formulations. Pharmaceutical formulations according to the present invention comprise a combination according to the invention together with one or more pharmaceutically acceptable carriers or excipients and optionally other therapeutic agents.

An inventive combination of one or more agents for increasing blood brain barrier permeability and one or more therapeutic agents may conveniently be presented as a pharmaceutical formulation in a unitary dosage form. A convenient unitary dosage formulation contains the active ingredients in amounts from 0.1 mg to 1 g each, for example 5 mg to 500 mg. Typical unit doses may, for example, contain about 0.5 to about 500 mg, or about 1 mg to about 500 mg of an agent for increasing blood brain barrier permeability. Other suitable dosages are set forth in Table 1, above.

According to the present invention, combinations of one or more agents for increasing blood brain barrier permeability and one or more therapeutic agents may be formulated for any mode of delivery including, for example, oral, rectal, nasal, topical (including transdermal, buccal and sublingual), vaginal or parenteral (including subcutaneous, intramuscular, intravenous and intradermal) administration. The formulations may be prepared by any methods well known in the art of pharmacy, for example, using methods such as those described in Gennaro et al., Remington's Pharmaceutical Sciences (18th ed., Mack Publishing Company, 1990, see especially Part 8: Pharmaceutical Preparations and their Manufacture). Such methods typically include a step of bringing into association the active ingredient(s) with the carrier which constitutes one or more accessory ingredients. Such accessory ingredients include, for example, fillers, binders, diluents, disintegrants, lubricants, colorants, flavouring agents and wetting agents.

Formulations suitable for oral administration may be presented, for example, as discrete units such as pills, tablets or capsules each containing a predetermined amount of active ingredient; as a powder or granules; as a solution or suspension. The active ingredient may also be present as a bolus or paste, or may be contained within liposomes.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Therapeutic agents and combinations of the present invention can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Formulations suitable for oral administration may alternatively be presented, for example, as liquids. Liquid formulations may be particularly useful for administration to children. In general, when preparing liquid formulations for administration to children, it is desirable to avoid or minimize use of alcohol in the formulation. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Formulations for rectal administration may be presented, for example, as a suppository or enema. Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

For parenteral administration, suitable formulations include aqueous and non-aqueous sterile injection. The formulations may be presented in unit-dose or multi-dose containers, for example, sealed vials and ampoules, and may be stored in a freeze dried (lyophilized) condition requiring only the addition of the sterile liquid carrier, for example, water prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound utilized in a provided combination therapy, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Dosage forms for topical or transdermal administration of a compound for use in combination therapy of the present invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Formulations suitable for administration by nasal inhalation include, for example, fine dusts or mists which may be generated by means such as metered dose pressurized aerosols, nebulisers or insufflators.

According to the present invention, pharmaceutical formulations may be prepared as “patient packs” containing the whole course of treatment in a single package, for example a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the inventive combination by means of a single patient pack, or patient packs of each formulation, with a package insert directing the patient to the correct use of the invention is a desirable additional feature of this invention.

According to a further aspect of the invention, there is provided a patient pack comprising at least one active ingredient of the combination of the invention and an information insert containing directions on the use of the combination of the invention. In other embodiments, the present invention provides a patient pack comprising both active ingredients of the combination of the invention for simultaneous or sequential administration to a patient, and further comprising an information insert containing directions on the use of the combination of the invention. In certain embodiments, the present invention provides a patient pack comprising both active ingredients of the combination of the invention formulated into a single unit dosage form for administration to a patient, and further comprising an information insert containing directions on the use of the combination of the invention.

### 5. Combination Products and Combined Administration

It will also be appreciated that provided agents which increase blood brain barrier permeability can be administered concurrently with, prior to, or subsequent to, one or more other desired therapeutics or medical procedures. Particular combination therapies (therapeutics or procedures) to employ in a combination regimen will take into account compatibility of the desired therapeutics and/or procedures and the desired therapeutic effect to be achieved. It will also be appreciated that therapies employed may achieve a desired effect for the same disorder (for example, a formulation may be administered concurrently with another compound used to treat the same disorder), or they may achieve different effects (e.g., control of any adverse effects). As used herein, additional therapeutic compounds which are normally administered to treat or prevent a particular disease, or condition, are known as “appropriate for the disease, or condition, being treated”.

An agent which increases blood brain barrier permeability and the therapeutic agent may be administered simultaneously, in the same or different pharmaceutical formulation, or sequentially. The timing of the sequential administration should preserve the advantageous effects of the combination and said timing can be determined by a skilled practitioner. In other embodiments, the combinations are combined in a single unit dosage form.

A therapeutically effective amount of the combination will be understood to be an amount which treats, inhibits, prevents or ameliorates one or more symptoms of the CNS disorder or episode in question. In certain embodiments of the invention, the combination will show improved efficacy than that achieved by administration of the same amount of the therapeutic agent alone. Furthermore, in certain embodiments the effective amount of the combination produces fewer side effects than are observed when the therapeutic agent is administered alone at a dose that achieves substantially similar therapeutic efficacy. Additionally, in certain embodiments, the effective amount of the combination results in increased therapeutic efficacy and a reduced effective dose of the therapeutic agent than is observed when the therapeutic agent is administered alone.

The dosages of each of the drugs in the combination may be determined by a physician and will often depend upon the specific disease or disorder, as well as the size, age and response pattern of the patient. Dosage guidelines are provided here. For the combination, the dosage guideline for each of the drugs of the combination would be considered.

In general, suitable doses of the agent which increases blood brain barrier permeability range from about 0.1 mg per day to about 1000 mg per day; in some embodiments from about 1 to about 500 mg per day.

A suitable dose of therapeutic agent may be in the range recommended by the manufacturer. In some embodiments of the invention, the therapeutic agent is used at the low end of the range recommended by the manufacturer, or even below the range, in light of the improved administration of therapeutic agent that can be achieved according to the present invention. Exemplary dosages for some therapeutic agents are provided as guidelines in Table 1.

As described above and herein, specific dosages of a provided combination can be based upon known and typical dosage ranges known for the particular agent utilized in the combination. However, and without wishing to be bound by any particular theory, it is believed that by administering a therapeutic agent for treating a CNS disease and/or disorder in combination with an agent that increases BBB permeability, in accordance with the present invention, the therapeutically effective amount of the agent will be lower than when administering the same therapeutic agent alone. In some embodiments, a therapeutically effective dosage of the therapeutic agent administered in a combination therapy of the present invention will be 90% of the typical dosage amount administered for the agent. In certain embodiments, a therapeutically effective dosage of the therapeutic agent administered in a combination therapy of the present invention will be 85, 80, 75, 70, 65, 60, 55, 50, 45, 40, 35, 30, 25, 20, 15, 10, or 5% of the typical dosage amount administered for the agent as compared with a therapeutically effective amount of the agent administered alone (i.e., not in a provided combination).

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebir®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

### 6. Uses

A combination therapy in accordance with the present invention comprises an agent to increase the BBB permeability and allow therapeutic agents to enter the CNS. The present invention provides a method of treating diseases and/or disorders of the CNS by administering to a subject (a) an agent for increasing blood brain barrier permeability, in combination with (b) a therapeutic agent for treating the disease or disorder. Such CNS disorders are described in greater detail below.

In certain embodiments, the present invention provides for a method of treating Acid Lipase disease, comprising administering one or more agents that increase BBB permeability in combination with one or more therapeutic agents. In one such embodiment, such therapeutic agent is lypolytic enzyme.

In certain embodiments, the present invention provides for a method of treating ADHD, comprising administering one or more agents that increase BBB permeability in combination with one or more therapeutic agents. Such therapeutic agents are selected from a group consisting of methylphenidate, dexmethylphenidate, amphetamine-dextroamphetamine, lisdexamfetamine, AFX 221, amfetamine, aripiprazole, AZD 1446, clonidine, eltoprazine, GTS 21, ispronicline, KRL 401, LY 2216684, MK 0249, ORG 26576, pozanicline, SGS 742, sofinicline and SPN 811.

In certain embodiments, the present invention provides for a method of treating Alzheimer's disease, comprising administering one or more agents that increase BBB permeability in combination with one or more therapeutic agents. In some embodiments, such therapeutic agents mediate Alzheimer's disease through the inhibition of cholinesterase or Amyloid Precursor Protein (APP), the regulation of Presenilin 1, Presenilin 2, and/or BACE. In other embodiments, such therapeutic agents are NMDA agonists. Exemplary cholinesterase inhibitors are selected from a group consisting of donepezil, galantamine, and rivastigmine. Exemplary NMDA antagonists include memantine. In some embodiments, such therapeutic agents are EPOE, ABT 126, Exebryl-1®, PeptiClere, ASP 0777, Atorvastatin, ¹⁸F-AV 1, ¹⁸F-V 45, AV 965, AVN 101, AZD 103, AZD 4694, Begacestat, Bisnorcymserine, BMS 708163, CERE 110, CHF 074, Conjugated estrogens, CX 717, Davunetide, DEBIO 9902, Dimebolin, Docosahexanoic acid, E 2012, EGb 761, ELND 006, EVP 0334, EVP 6124, HPP 854, Huperzine A, Immunoglobulin, Indolepropionic acid derivatives, LY 2811376, LY 451395, MABT 5102A, MCD 386, MEM 1003, MEM 1414, MEM 3454, MK 0249, NGX 267, NIC 515, Nicergoline, NSA 789, PF 04494700, PF 3654764, Phenserine, Pittsburgh compound B, Pozanicline, PRX 03140, PRX 07034, R-phenserine, AFX 929, RN 1219, RVX 208, SAM 531, SB 742457, Semagacestat, SGS 742, T 817MA and V 950.

In certain embodiments, the present invention provides for a method of treating anxiety disorders, comprising administering one or more agents that increase BBB permeability in combination with one or more therapeutic agents. In some embodiments, such therapeutic agents are selected from a group consisting of alprazolam, clonazepam, diazepam, escitalopram, fluoxetine, gabapentin, hydroxyzine, imipramine, paroxetine, phenelzine, piperazines, pregabalin, sertraline, tranylcypromine, venlafaxine, ADX 71149, AST 117, AZD 2327, AZD 7268, AZD 7325, KP 157, Emicerfont, GABA A receptor agonists, GSK 586529, GSK 588045, Midazolam, PH 94B, SPN 805 and YKP 3089.

In certain embodiments, the present invention provides for a method of treating Barth syndrome, comprising administering one or more agents that increase BBB permeability in combination with one or more therapeutic agents. In one such embodiment, such therapeutic agent is acyltransferase.

In certain embodiments, the present invention provides for a method of treating bipolar disorder, comprising administering one or more agents that increase BBB permeability in combination with one or more therapeutic agents. In some embodiments, such therapeutic agents are selected from a group consisting of lithium carbonate, lamotrigine, sodium valproate, carbamazepine, quetiapine, chlorpromazine, topiramate, armodafinil and PF 4455242.

In certain embodiments, the present invention provides for a method of treating cancer, comprising administering one or more agents that increase BBB permeability in combination with one or more therapeutic agents. In some embodiments, such therapeutic agents mediate brain cancer through the regulation of hormones, inhibition of angiogenesis, inhibition of kinases, inhibition of metabolism, inhibition of topoisomerases. In some embodiments, such therapeutic agents are alkylating antineoplastic agents or antitumor antibiotics. Exemplary angiogenesis inhibitors include bevacizumab. Exemplary kinase inhibitors include imatinib, temozolomide and gefitinib. Exemplary metabolism inhibitors include azathiopurine, mercaptopurine, yinca alkaloids, taxanes and podophyllotoxin. Exemplary topoisomerase inhibitors include irinotecan, topotecan, amsacrine and etoposide. Exemplary alkylating antineoplastic agents include cisplatin, carboplatin, oxaplatin, mechlorethamine, cyclophosphamide, chlorambucil and ifosamide. Exemplary antitumor antibiotics include dactinomycin, doxorubicin, epirubicin and bleomycin. In some embodiments, the therapeutic agent is selected from a group consisting of interferons, vesicular stomatitis virus, trastuzumab, rituximab 17-AAG, AFP-scan, AFX 9901, AGS 16M18, aldesleukin, ALT 801, AMG 479, antibody-drug conjugates, antineoplaston A10, antineoplaston AS2-1, arginine butyrate, ARRY 300, AVR 118, bleotecan, BIO 109, BIO 113, BLX 883, BMS188797, BMS 310705, BMS 663513, calcitriol, pDNA cancer vaccine, cancer vaccines, carbendazim, CLT 001, CNDO 101, CPI 613, CS 7017, CZ 112, docetaxel, E 7820, EC 0225, EC 20, ENMD 1198, epirubicin, etoposide, F 50035, GDC 0152, GI 6207, GSK 1059615, GSK 1120212, GSK 2126458, IC 83, IGN 301, IMC 18F1, In 111 DAC, ixabepilone, KW 2450, KX2 391, LBY 135, LR 103, LY 2157299, LY 2523355, milataxel, MK 0752, MK 4101, MK 8033, MK 2461, MLN 9708, monoclonal antibody 3F8, NRX 4204, OPB 31121, OSI 7904L, palifosfamide, PBI 05204, PCI-27483, PD 332991, PF 3084014, PF 337210, PF 3446962, PF 3732010, PF 4217903, PF 4554878, PF 562271, PHA 848125, prednimustine, PX 12, QBI 139, RDEA 119, SG 2000, sirolimus, SNX 5422, TAK 285, TAK 701, TRC 105, veglin, VTX 2337, XL 139, XL 184, XL 228, YM 155, ZYC 300, DM-CHOC-PEN, AEE 788, AT 101, banoxantrone, benzylguanine, bevacizumab, BIBW 2992, brain cancer vaccines, BSI 201, CC 8490, CDX 110, cilengitide, cintredekin besudotox, contusugene ladenovec, CT 322, enzastaurin, erlotinib, interleukin-4(38-37)-PE38 KDEL, lenalidomide, lonafarnib, monoclonal antibody TNT-1, motexafin gadolinium, MPC 6827, poly ICLC, sodium phenylbutyrate, tandutinib and vorinostat.

In certain embodiments, the present invention provides for a method of treating borderline personality disorder, comprising administering one or more agents that increase BBB permeability in combination with one or more therapeutic agents. In some embodiments, such therapeutic are selected from a group consisting of atypical antipsychotics, antipsychotics, olanzapine, clozapine, quetiapine, risperidone, lithium carbonate and lamotrigine.

In certain embodiments, the present invention provides for a method of treating Canavan disease, comprising administering one or more agents that increase BBB permeability in combination with one or more therapeutic agents. In one such embodiment, such therapeutic agent is aspartoacyclase enzyme.

In certain embodiments, the present invention provides for a method of treating Dawson disease, comprising administering one or more agents that increase BBB permeability in combination with one or more therapeutic agents. In some embodiments, such therapeutic agents are antiviral agents.

In certain embodiments, the present invention provides for a method of treating depression, comprising administering one or more agents that increase BBB permeability in combination with one or more therapeutic agents. In some embodiments, such therapeutic agents mediate depression through selective serotonin reuptake inhibition or MAO inhibition. Exemplary SSRIs include escitalopram, fluoxetine, paroxetine, citalopram, bupropion and venlafaxine. Exemplary MAO inhibitors include selegiline, rasagiline, protriptyline, imipramine and clomipramine 1n other embodiments, such therapeutic agents are selected from the group consisting of ADX N05, agomelatine, AZD 2327, AZD 6765, AZD 7268, buspirone/melatonin, cariprazine, calvulanic acid, CPI 300, CX 157, desvenlafaxine, duloxetine, emicerfont, GSK 586529, GSK 588045, lisdexamfetamine, LU AA 21004, LY 2216684, mifepristone, nefiracetam, nemifitide, omega-3 ethylester, ORG 26576, ORG 34517, orvepitant, pexacerfont, reboxetine, quetiapine, risperidone, SEP 225289, SEP 227162, SEP 228432, tasimelteon, TC 5214, TGBA01AD, traxoprodil, trazodone, venlafaxine, deuterated venlafaxine, verucerfont and vilazodone.

In certain embodiments, the present invention provides for a method of treating eating disorders, comprising administering one or more agents that increase BBB permeability in combination with one or more therapeutic agents. In some embodiments, such therapeutic agents include fluoxetine and paroxetine.

In certain embodiments, the present invention provides for a method of treating Fabry disease, comprising administering one or more agents that increase BBB permeability in combination with one or more therapeutic agents. In one such embodiment, such therapeutic is alpha-galactosidase A.

In certain embodiments, the present invention provides for a method of treating Hallervorden-Spatz disease, comprising administering one or more agents that increase BBB permeability in combination with one or more therapeutic agents. In one such embodiment, such therapeutic is pantothenate kinase 2.

In certain embodiments, the present invention provides for a method of treating headache, comprising administering one or more agents that increase BBB permeability in combination with one or more therapeutic agents. In some embodiments, such therapeutic agents are selected from a group consisting of acetaminophen, acetylsalicylic acid, diclofenac and ibuprofen.

In certain embodiments, the present invention provides for a method of treating HIV, comprising administering one or more agents that increase BBB permeability in combination with one or more therapeutic agents. In some embodiments, such therapeutic agents are selected from the group consisting of nelfinavir, ADVAX, AMZ 0026, anti-CCRS monoclonal antibodies, ATI 0917, BAY 504798, carbendazim, dapivirine, elvitegravir/emtricitabine/tenofovir disoproxil fumarate/GS 9350, elvucitabine, emtricitabine/rilpivirine/tenofovir disoproxil fumarate, GSK 1247303, GSK 1265744, HIV adrenovector serotype Ad35 vaccine, HIV combination vaccines, HIV DNA vaccines, rgp120(SF2)/MF59 vaccine, ibalizumab, INCB 15050, INCB 9471, interferon-alpha-3, interleukin-7, KP 1461, lexgenleucel-T, lopinavir/ritonavir, nonakine, PBS119, peginterferon alpha-2a, PRO2000, procaine, PSI 5004, SP 01A, SB-728-T, SPD 756, SPI 256, SPL 7013, thalidomide, TR 291144, UC 781, V 526 and VRC-HIVADV014-00-VP.

In certain embodiments, the present invention provides for a method of treating Huntington's disease, comprising administering one or more agents that increase BBB permeability in combination with one or more therapeutic agents. In some embodiments, such therapeutic agents are selected from a group consisting of tetrabenazine, valproic acid, SSRIs, atypical antipsychotics, amantadine and remacemide.

In certain embodiments, the present invention provides for a method of treating Lewy Body disease, comprising administering one or more BBB modulators in combination with one or more therapeutic agents. In some embodiments, such therapeutic agents mediate Lewy Body disease through inhibition of cholinesterases. Exemplary cholinesterase inhibitors include donepezil, rivastigmine, galantamine, Sinemet®, clonazepam, methylphenidate, modafinil and riluzole.

In certain embodiments, the present invention provides for a method of treating Lou Gehrig's disease, comprising administering one or more agents that increase BBB permeability in combination with one or more therapeutic agents. In some embodiments, such therapeutic agents mediate Lou Gehrig's disease through ion channel blocking or the inhibition of protein kinase C. Exemplary protein kinase C inhibitors include arimoclomol, IGF-1, minocycline and KNS-760704. Other exemplary therapeutic agents which treat Lou Gehrig's disease are selected from the group consisting of AEOL 10150, Arimoclomol, Creatine monohydrate, Mecasermin rinfabate, NEU 2000, Olesoxime, PYM 50018 and SB 509.

In certain embodiments, the present invention provides for a method of treating Machado-Joseph disease, comprising administering one or more agents that increase BBB permeability in combination with one or more therapeutic agents. In some embodiments, such therapeutic agents include baclofen or levodopa.

In certain embodiments, the present invention provides for a method of treating narcolepsy, comprising administering one or more agents that increase BBB permeability in combination with one or more therapeutic agents. In some embodiments, such therapeutic agents mediate narcolepsy through the inhibition of norepinephrine reuptake. Exemplary norepinephrine reuptake inhibitors include atomoxetine, clomipramine, codeine, dextroamphetamine, gamma hydroxybutyrate, imipramine, methamphetamine, methylphenidate, modafinil, protriptyline, selegiline, KRL 102, CX 717, melatonin or tricyclic antidepressants.

In certain embodiments, the present invention provides for a method of treating obsessive-compulsive disorder, comprising administering one or more agents that increase BBB permeability in combination with one or more therapeutic agents. In some embodiments, such therapeutic agents are selected from a group consisting of atypical antidepressants, benzodiazepines, carbamazepine, chlorpromazine, escitalopram, fluoxetine, lamotrigine, N-acetylcysteine, olanzapine, paroxetine, quetiapine, topiramate, cycloserine, elzasonan, NPL 2003, and tricyclic antidepressants.

In certain embodiments, the present invention provides for a method of treating pain, comprising administering one or more agents that increase BBB permeability in combination with one or more therapeutic agents. In some embodiments, such therapeutic agents are selected from a group consisting of NSAIDs, COX-2 inhibitors, morphine, codeine, hydrocodone, diamorphine, meperidine, tramadol, buprenorphine, amitriptyline, paracetamol, ibuprofen, naproxen, opiates, CJ 15161, dronabinol/cannabidiol, fentanyl, JNJ 42160443, ketamine, NP 2, pamidronic acid, sufentanil, tanezumab, ALKS 33, ecopipam, isovaleramide, pivagabine esters, botulinum toxin A, CGRP antagonists, COL 144, dihydroergotamine, donepezil, FHPC 01, gabapentin, lacosamide, loxapine, LY 466195, naproxen sodium/sumatriptan, NGX 426, olcegepant, prochlorperazine, sumatriptan, telecagepant, tezampanel, tonabersat, ABT 102, ADL 5747, AGN 203818, AGN 323, ALGRX 4975, AMG 379, AMG 403, aspirin/omeprazole, aspirin/phosphatidylcholine, AZD 1386, bupivacaine, buprenorphine, capsaicin, CEP 28190, CPL 7075, DDS, dexmedetomidine, diclofenac, DPI 125, hydromorphone, ibuprofen/famotidine, ICA 105665, JNJ 38488502, ketoprofen, L 791515, lidocaine. LPCN 1029, MGX 001, MK 4409, morphine-6-glucuronide, morphine/oxycodone, oxycodone, oxycodone/naltrexone, oxycodone/niacin, PF 4136309, PF 3557156, PF 4191834, PF 4457845, PF 4856880, PF 4856881, PLX 5568, pregabalin, PTI 202, PTI 721, radiprodil, recombinant clostriadial neurotoxin protease, REGN 475, RPI 70, SAB 378, SCP 1, sufentanil/triazolam, SYN 116, tapentadol, thalidomide, URG 301, vanilloid receptor antagonists, zucapsaicin and topiramate.

In certain embodiments, the present invention provides for a method of treating Parkinson's disease, comprising administering one or more agents that increase BBB permeability in combination with one or more therapeutic agents. In some embodiments, such therapeutic agents mediate Parkinson's disease through the inhibition of dopa carboxylase, inhibition of COMT, inhibition of MAO-B, inhibition of alpha-synuclein, inhibition of LRRK2, inhibition of DJ-1. In other embodiments, Parkinson's disease is mediated by dopamine agonists or by delivery of Parkin or Pink1. Exemplary dopa carboxylase inhibitors include carbidopa, benserazide, cardidopa/levodopa, and benserazide/levodopa. Exemplary COMT inhibitors include tolcapone. Exemplary MAO-B inhibitors include selegine, rasagiline, amantadine, benzotropine, trihexyphenidyl, selegiline and entacapone. Exemplary dopamine agonists include bromocriptine, pergolide, pramipexole, ropinirole, piribedil, cabergoline, apomorphine and lisuride. In other embodiments, such therapeutic agents are selected from the group consisting of aplindore, apomorphine, autologous stem cell therapy, AV 201, AV 45, CERE 120, creatine, DAR 100, fipamezole, ioflupane 1231, MK 0657, NLX P101, nitisinone, preladenant, rotigotine, safinamide, SPN 803, SYN 115, traxoprodil, ubidecarenone, V 1512 and XP 21279.

In certain embodiments, the present invention provides for a method of treating Restless Leg syndrome, comprising administering one or more agents that increase BBB permeability in combination with one or more therapeutic agents. In some embodiments, such therapeutic agents are selected from a group consisting of ropinirole, pramipexole, rotigotine, opioids, aplindore, gabapentin enacarbil, pregabalin and benzodiazepines.

In certain embodiments, the present invention provides for a method of treating schizophrenia, comprising administering one or more agents that increase BBB permeability in combination with one or more therapeutic agents. In some embodiments, such therapeutic agents are selected from a group consisting of amisulpride, aripiprazole, asenapine, chlorpromazine, chlorprothixene, clozapine, droperidol, flupenthixol, fluphenazine, haloperidol, iloperidone, levomepromazine, mesoridazine, olanzapine, paliperidone, periciazine, perphenazine, pimozide, prochlorperazine, promazine, promethazine, quetiapine, risperidone, sertindole, thioridazine, thiothixene, trifluoperazine, triflupromazine, ziprasidone, zotepine, adipiplon, ADX 71149, armodafinil, ATI 9242, AVN 211, AZD 8529, BL 1020, cariprazine, CM 2395, davunetide, DCCCyB, EVP 6124, GSK 1144814, idazoxan, iloperidone, ITI 007, JNJ 17305600, lisdexamfetamine, loxapine, lurasidone, MEM 3454, MK 0249, NSA 789, ocaperidone, ORG 25935, paliperidone, PF 217830, PF 2545920, PF 3463275, pimavanserin, R 1678, sabcomeline, SB 773812, TGOFO2N, tiprolisant and zuclopenthixol. In another embodiment, schizophrenia is mediated by mGluR2 agonists.

In certain embodiments, the present invention provides for a method of treating seizure disorders, comprising administering one or more agents that increase BBB permeability in combination with one or more therapeutic agents. In some embodiments, such therapeutic agents are selected from a group consisting of beclamide, brivaracetam, carbamazepine, carbatrol, clobazam, diastat, ethosuximide, felbamate, fosphenyloin, gabapentin, hydantoins, inovelon, lamotrigine, levetiracetam, mesuximide, neurotrin, nitrazepam, phenacemide, pheneturide, phenobarbitol, phenyloin, pregabalin, primidone, pyrimidindiones, vigabatrin, stiripentol, temazepam, tiagabine, topiramate, trileptal, valnoctamide, valproic acid, valproamide, lacosamide and zonisamide.

In certain embodiments, the present invention provides for a method of treating Tourette's syndrome, comprising administering one or more agents that increase BBB permeability in combination with one or more therapeutic agents. In some embodiments, such therapeutic agents are selected from a group consisting of typical antipsychotics, atypical psychotics, fluphenazine, haloperidol, pimozide, risperidonen ziprasidone and AFX 221.

In certain embodiments, the present invention provides for a method of treating viral infections, comprising administering one or more agents that increase BBB permeability in combination with one or more therapeutic agents. In some embodiments, such therapeutic agents are selected from a group consisting of arginine butyrate, famiciclovir, tenofovir, zanamivir, oseltamivir, valomaciclovir and valacyclovir.

In certain embodiments, the present invention provides for a method of treating Wernicke-Korsakoff syndrome, comprising administering one or more agents that increase BBB permeability in combination with one or more therapeutic agents. In one embodiment, such therapeutic agent is thiamine.

In certain embodiments, the present invention provides for a method of treating stroke, comprising administering one or more agents that increase BBB permeability in combination with one or more therapeutic agents. Such therapeutic agents are selected from a group consisting of apixaban, aspirin/phosphatidylcholine, betrixaban, BVI 007, desmoteplase, MK 0724, MP 124, NA 1, NEU 2000, oxygenated fluorocarbin nutrient emulsion, rivaroxaban, SUN N8075, tenecteplase, traxoprodil, TS 011, V 10153 and zonampanel.

In certain embodiments, the present invention provides for a method of treating personality disorders, comprising administering one or more agents that increase BBB permeability in combination with one or more therapeutic agents. Such therapeutic agents are selected from a group consisting of ADX 71149, quetiapine and olanzapine.

In certain embodiments, the present invention provides for a method of treating post-traumatic stress disorders, comprising administering one or more agents that increase BBB permeability in combination with one or more therapeutic agents. Such therapeutic agents are selected from a group consisting of cycloserine, MDMA, mirtazapine, nepicastat, topiramate and MK 0594.

In certain embodiments, the present invention provides for a method of treating panic disorders, comprising administering one or more agents that increase BBB permeability in combination with one or more therapeutic agents. Such therapeutic agents are selected from a group consisting of cycloserine, escitalopram and ORG 25935.

In certain embodiments, the present invention provides for a method of providing neuroprotection, comprising administering one or more agents that increase BBB permeability in combination with one or more therapeutic agents. In one such embodiment, the therapeutic agent is epoetin alfa.

In certain embodiments, the present invention provides for a method of treating neurological disorders, comprising administering one or more agents that increase BBB permeability in combination with one or more therapeutic agents. Such therapeutic agents are selected from a group consisting of A 0001, ABT 384, amantadine, KP 544, MK 5395, ORG 26041, ORG 50189, triacetyluridine and ubidecarenone.

In certain embodiments, the present invention provides for a method of treating neurodegenerative disorders, comprising administering one or more agents that increase BBB permeability in combination with one or more therapeutic agents. Such therapeutic agents include AV 133 and OSI 754.

In certain embodiments, the present invention provides for a method of treating female sexual dysfunction, comprising administering one or more agents that increase BBB permeability in combination with one or more therapeutic agents. Such therapeutic agents are selected from a group consisting of bremelanotide, flibanserin and testosterone.

In certain embodiments, the present invention provides for a method of treating cognition disorders, comprising administering one or more agents that increase BBB permeability in combination with one or more therapeutic agents. Such therapeutic agents are selected from a group consisting of ABT 560, AV 965, DAR 100, HTC 867, IPL 455903, levafetamine, LU AE 58054, nefiracetam, PF 3654746, PF 4447943, phenserine, PRX 07034, R-phenserine, SYN 114 and SYN 120.

In certain embodiments, the present invention provides for a method of treating CNS disorders, comprising administering one or more agents that increase BBB permeability in combination with one or more therapeutic agents. Such therapeutic agents are selected from a group consisting of ecopipam, isovaleramide, pivagabine esters, GSK 249320 and ALKS 33.

In certain embodiments, the present invention provides for a method of treating dementia, comprising administering one or more agents that increase BBB permeability in combination with one or more therapeutic agents. Such therapeutic agents are selected from a group consisting of ABT 560, ADS 8703 and nefiracetam.

In certain embodiments, the present invention provides for a method of treating Asperger's syndrome, comprising administering one or more agents that increase BBB permeability in combination with one or more therapeutic agents. One such therapeutic agent is aripiprazole.

In certain embodiments, the present invention provides for a method of treating an autoimmune disorder, comprising administering one or more agents that increase BBB permeability in combination with one or more therapeutic agents. Such therapeutic agents are selected from a group consisting of PRTX 100, semapimod, SGN 70 and VBY 129.

In some embodiments, the present invention provides for a method of treating a CNS disease in a subject, comprising the step of administering to said subject (a) an agent for increasing blood brain barrier permeability in a subject, in combination with (b) a pharmaceutical agent for treating the disease or disorder. Such CNS diseases are described in greater detail below.

In certain embodiments, the present invention provides for a method of treating Acid Lipase disease, comprising administering one or more agents that increase BBB permeability in combination with one or more therapeutic agents. In one such embodiment, such therapeutic agent is lypolytic enzyme.

In certain embodiments, the present invention provides for a method of treating ADHD, comprising administering one or more agents that increase BBB permeability in combination with one or more therapeutic agents. Such therapeutic agents are selected from a group consisting of methylphenidate, dexmethylphenidate, amphetamine-dextroamphetamine, lisdexamfetamine, AFX 221, amfetamine, aripiprazole, AZD 1446, clonidine, eltoprazine, GTS 21, ispronicline, KRL 401, LY 2216684, MK 0249, ORG 26576, pozanicline, SGS 742, sofinicline and SPN 811.

In certain embodiments, the present invention provides for a method of treating Alzheimer's disease, comprising administering one or more agents that increase BBB permeability in combination with one or more therapeutic agents. In some embodiments, such therapeutic agents mediate Alzheimer's disease through the inhibition of cholinesterase or Amyloid Precursor Protein (APP), the regulation of Presenilin 1, Presenilin 2, and/or BACE. In other embodiments, such therapeutic agents are NMDA agonists. Exemplary cholinesterase inhibitors are selected from a group consisting of donepezil, galantamine, and rivastigmine. Exemplary NMDA antagonists include memantine. In some embodiments, such therapeutic agents are EPOE, ABT 126, Exebryl-1®, PeptiClere, ASP 0777, Atorvastatin, ¹⁸F-AV 1, ¹⁸F-V 45, AV 965, AVN 101, AZD 103, AZD 4694, Begacestat, Bisnorcymserine, BMS 708163, CERE 110, CHF 074, Conjugated estrogens, CX 717, Davunetide, DEBIO 9902, Dimebolin, Docosahexanoic acid, E 2012, EGb 761, ELND 006, EVP 0334, EVP 6124, HPP 854, Huperzine A, Immunoglobulin, Indolepropionic acid derivatives, LY 2811376, LY 451395, MABT 5102A, MCD 386, MEM 1003, MEM 1414, MEM 3454, MK 0249, NGX 267, NIC 515, Nicergoline, NSA 789, PF 04494700, PF 3654764, Phenserine, Pittsburgh compound B, Pozanicline, PRX 03140, PRX 07034, R-phenserine, AFX 929, RN 1219, RVX 208, SAM 531, SB 742457, Semagacestat, SGS 742, T 817MA and V 950.

In certain embodiments, the present invention provides for a method of treating anxiety disorders, comprising administering one or more agents that increase BBB permeability in combination with one or more therapeutic agents. In some embodiments, such therapeutic agents are selected from a group consisting of alprazolam, clonazepam, diazepam, escitalopram, fluoxetine, gabapentin, hydroxyzine, imipramine, paroxetine, phenelzine, piperazines, pregabalin, sertraline, tranylcypromine, venlafaxine, ADX 71149, AST 117, AZD 2327, AZD 7268, AZD 7325, KP 157, Emicerfont, GABA A receptor agonists, GSK 586529, GSK 588045, Midazolam, PH 94B, SPN 805 and YKP 3089.

In certain embodiments, the present invention provides for a method of treating Barth syndrome, comprising administering one or more agents that increase BBB permeability in combination with one or more therapeutic agents. In one such embodiment, such therapeutic agent is acyltransferase.

In certain embodiments, the present invention provides for a method of treating bipolar disorder, comprising administering one or more agents that increase BBB permeability in combination with one or more therapeutic agents. In some embodiments, such therapeutic agents are selected from a group consisting of lithium carbonate, lamotrigine, sodium valproate, carbamazepine, quetiapine, chlorpromazine, topiramate, armodafinil and PF 4455242.

In certain embodiments, the present invention provides for a method of treating a cancer, comprising administering one or more agents that increase BBB permeability in combination with one or more therapeutic agents. In some embodiments, such therapeutic agents mediate brain cancer through the regulation of hormones, inhibition of angiogenesis, inhibition of kinases, inhibition of metabolism, inhibition of topoisomerases. In some embodiments, such therapeutic agents are alkylating antineoplastic agents or antitumor antibiotics. Exemplary angiogenesis inhibitors include bevacizumab. Exemplary kinase inhibitors include imatinib, temozolomide and gefitinib. Exemplary metabolism inhibitors include azathiopurine, mercaptopurine, vinca alkaloids, taxanes and podophyllotoxin. Exemplary topoisomerase inhibitors include irinotecan, topotecan, amsacrine and etoposide. Exemplary alkylating antineoplastic agents include cisplatin, carboplatin, oxaplatin, mechlorethamine, cyclophosphamide, chlorambucil and ifosamide. Exemplary antitumor antibiotics include dactinomycin, doxorubicin, epirubicin and bleomycin. In some embodiments, the therapeutic agent is selected from a group consisting of interferons, vesicular stomatitis virus, trastuzumab, rituximab 17-AAG, AFP-scan, AFX 9901, AGS 16M18, aldesleukin, ALT 801, AMG 479, antibody-drug conjugates, antineoplaston A10, antineoplaston AS2-1, arginine butyrate, ARRY 300, AVR 118, bleotecan, BIO 109, BIO 113, BLX 883, BMS 188797, BMS 310705, BMS 663513, calcitriol, pDNA cancer vaccine, cancer vaccines, carbendazim, CLT 001, CNDO 101, CPI 613, CS 7017, CZ 112, docetaxel, E 7820, EC 0225, EC 20, ENMD 1198, epirubicin, etoposide, F 50035, GDC 0152, GI 6207, GSK 1059615, GSK 1120212, GSK 2126458, IC 83, IGN 301, IMC 18F1, In 111 DAC, ixabepilone, KW 2450, KX2 391, LBY 135, LR 103, LY 2157299, LY 2523355, milataxel, MK 0752, MK 4101, MK 8033, MK 2461, MLN 9708, monoclonal antibody 3F8, NRX 4204, OPB 31121, OSI 7904L, palifosfamide, PBI 05204, PCI-27483, PD 332991, PF 3084014, PF 337210, PF 3446962, PF 3732010, PF 4217903, PF 4554878, PF 562271, PHA 848125, prednimustine, PX 12, QBI 139, RDEA 119, SG 2000, sirolimus, SNX 5422, TAK 285, TAK 701, TRC 105, veglin, VTX 2337, XL 139, XL 184, XL 228, YM 155, ZYC 300, DM-CHOC-PEN, AEE 788, AT 101, banoxantrone, benzylguanine, bevacizumab, BIBW 2992, brain cancer vaccines, BSI 201, CC 8490, CDX 110, cilengitide, cintredekin besudotox, contusugene ladenovec, CT 322, enzastaurin, erlotinib, interleukin-4(38-37)-PE38 KDEL, lenalidomide, lonafarnib, monoclonal antibody TNT-1, motexafin gadolinium, MPC 6827, poly ICLC, sodium phenylbutyrate, tandutinib and vorinostat.

In certain embodiments, the present invention provides for a method of treating borderline personality disorder, comprising administering one or more agents that increase BBB permeability in combination with one or more therapeutic agents. In some embodiments, such therapeutic are selected from a group consisting of atypical antipsychotics, antipsychotics, olanzapine, clozapine, quetiapine, risperidone, lithium carbonate and lamotrigine.

In certain embodiments, the present invention provides for a method of treating Canavan disease, comprising administering one or more agents that increase BBB permeability in combination with one or more therapeutic agents. In one such embodiment, such therapeutic agent is aspartoacyclase enzyme.

In certain embodiments, the present invention provides for a method of treating Dawson disease, comprising administering one or more agents that increase BBB permeability in combination with one or more therapeutic agents. In some embodiments, such therapeutic agents are antiviral agents.

In certain embodiments, the present invention provides for a method of treating depression, comprising administering one or more agents that increase BBB permeability in combination with one or more therapeutic agents. In some embodiments, such therapeutic agents mediate depression through selective serotonin reuptake inhibition or MAO inhibition. Exemplary SSRIs include escitalopram, fluoxetine, paroxetine, citalopram, bupropion and venlafaxine. Exemplary MAO inhibitors include selegiline, rasagiline, protriptyline, imipramine and clomipramine 1n other embodiments, such therapeutic agents are selected from the group consisting of ADX N05, agomelatine, AZD 2327, AZD 6765, AZD 7268, buspirone/melatonin, cariprazine, calvulanic acid, CPI 300, CX 157, desvenlafaxine, duloxetine, emicerfont, GSK 586529, GSK 588045, lisdexamfetamine, LU AA 21004, LY 2216684, mifepristone, nefiracetam, nemifitide, omega-3 ethylester, ORG 26576, ORG 34517, orvepitant, pexacerfont, reboxetine, quetiapine, risperidone, SEP 225289, SEP 227162, SEP 228432, tasimelteon, TC 5214, TGBA01AD, traxoprodil, trazodone, venlafaxine, deuterated venlafaxine, verucerfont and vilazodone.

In certain embodiments, the present invention provides for a method of treating an eating disorder, comprising administering one or more agents that increase BBB permeability in combination with one or more therapeutic agents. In some embodiments, such therapeutic agents include fluoxetine and paroxetine.

In certain embodiments, the present invention provides for a method of treating Fabry disease, comprising administering one or more agents that increase BBB permeability in combination with one or more therapeutic agents. In one such embodiment, such therapeutic is alpha-galactosidase A.

In certain embodiments, the present invention provides for a method of treating Hallervorden-Spatz disease, comprising administering one or more agents that increase BBB permeability in combination with one or more therapeutic agents. In one such embodiment, such therapeutic is pantothenate kinase 2.

In certain embodiments, the present invention provides for a method of treating headache, comprising administering one or more agents that increase BBB permeability in combination with one or more therapeutic agents. In some embodiments, such therapeutic agents are selected from a group consisting of acetaminophen, acetylsalicylic acid, diclofenac and ibuprofen.

In certain embodiments, the present invention provides for a method of treating HIV, comprising administering one or more agents that increase BBB permeability in combination with one or more therapeutic agents. In some embodiments, such therapeutic agents are selected from the group consisting of nelfinavir, ADVAX, AMZ 0026, anti-CCR5 monoclonal antibodies, ATI 0917, BAY 504798, carbendazim, dapivirine, elvitegravir/emtricitabine/tenofovir disoproxil fumarate/GS 9350, elvucitabine, emtricitabine/rilpivirine/tenofovir disoproxil fumarate, GSK 1247303, GSK 1265744, HIV adrenovector serotype Ad35 vaccine, HIV combination vaccines, HIV DNA vaccines, rgp120(SF2)/MF59 vaccine, ibalizumab, INCB 15050, INCB 9471, interferon-alpha-3, interleukin-7, KP 1461, lexgenleucel-T, lopinavir/ritonavir, nonakine, PBS119, peginterferon alpha-2a, PRO2000, procaine, PSI 5004, SP 01A, SB-728-T, SPD 756, SPI 256, SPL 7013, thalidomide, TR 291144, UC 781, V 526 and VRC-HIVADV014-00-VP.

In certain embodiments, the present invention provides for a method of treating Huntington's disease, comprising administering one or more agents that increase BBB permeability in combination with one or more therapeutic agents. In some embodiments, such therapeutic agents are selected from a group consisting of tetrabenazine, valproic acid, SSRIs, atypical antipsychotics, amantadine and remacemide.

In certain embodiments, the present invention provides for a method of treating Lewy Body disease, comprising administering one or more BBB modulators in combination with one or more therapeutic agents. In some embodiments, such therapeutic agents mediate Lewy Body disease through inhibition of cholinesterases. Exemplary cholinesterase inhibitors include donepezil, rivastigmine, galantamine, Sinemet®, clonazepam, methylphenidate, modafinil and riluzole.

In certain embodiments, the present invention provides for a method of treating Lou Gehrig's disease, comprising administering one or more agents that increase BBB permeability in combination with one or more therapeutic agents. In some embodiments, such therapeutic agents mediate Lou Gehrig's disease through ion channel blocking or the inhibition of protein kinase C. Exemplary protein kinase C inhibitors include arimoclomol, IGF-1, minocycline and KNS-760704. Other exemplary therapeutic agents which treat Lou Gehrig's disease are selected from the group consisting of AEOL 10150, Arimoclomol, Creatine monohydrate, Mecasermin rinfabate, NEU 2000, Olesoxime, PYM 50018 and SB 509.

In certain embodiments, the present invention provides for a method of treating Machado-Joseph disease, comprising administering one or more agents that increase BBB permeability in combination with one or more therapeutic agents. In some embodiments, such therapeutic agents include baclofen or levodopa.

In certain embodiments, the present invention provides for a method of treating narcolepsy, comprising administering one or more agents that increase BBB permeability in combination with one or more therapeutic agents. In some embodiments, such therapeutic agents mediate narcolepsy through the inhibition of norepinephrine reuptake. Exemplary norepinephrine reuptake inhibitors include atomoxetine, clomipramine, codeine, dextroamphetamine, gamma hydroxybutyrate, imipramine, methamphetamine, methylphenidate, modafinil, protriptyline, selegiline, KRL 102, CX 717, melatonin or tricyclic antidepressants.

In certain embodiments, the present invention provides for a method of treating obsessive-compulsive disorder, comprising administering one or more agents that increase BBB permeability in combination with one or more therapeutic agents. In some embodiments, such therapeutic agents are selected from a group consisting of atypical antidepressants, benzodiazepines, carbamazepine, chlorpromazine, escitalopram, fluoxetine, lamotrigine, N-acetylcysteine, olanzapine, paroxetine, quetiapine, topiramate, cycloserine, elzasonan, NPL 2003, and tricyclic antidepressants.

In certain embodiments, the present invention provides for a method of treating pain, comprising administering one or more agents that increase BBB permeability in combination with one or more therapeutic agents. In some embodiments, such therapeutic agents are selected from a group consisting of NSAIDs, COX-2 inhibitors, morphine, codeine, hydrocodone, diamorphine, meperidine, tramadol, buprenorphine, amitriptyline, paracetamol, ibuprofen, naproxen, opiates, CJ 15161, dronabinol/cannabidiol, fentanyl, JNJ 42160443, ketamine, NP 2, pamidronic acid, sufentanil, tanezumab, ALKS 33, ecopipam, isovaleramide, pivagabine esters, botulinum toxin A, CGRP antagonists, COL 144, dihydroergotamine, donepezil, FHPC 01, gabapentin, lacosamide, loxapine, LY 466195, naproxen sodium/sumatriptan, NGX 426, olcegepant, prochlorperazine, sumatriptan, telecagepant, tezampanel, tonabersat, ABT 102, ADL 5747, AGN 203818, AGN 323, ALGRX 4975, AMG 379, AMG 403, aspirin/omeprazole, aspirin/phosphatidylcholine, AZD 1386, bupivacaine, buprenorphine, capsaicin, CEP 28190, CPL 7075, DDS, dexmedetomidine, diclofenac, DPI 125, hydromorphone, ibuprofen/famotidine, ICA 105665, JNJ 38488502, ketoprofen, L 791515, lidocaine. LPCN 1029, MGX 001, MK 4409, morphine-6-glucuronide, morphine/oxycodone, oxycodone, oxycodone/naltrexone, oxycodone/niacin, PF 4136309, PF 3557156, PF 4191834, PF 4457845, PF 4856880, PF 4856881, PLX 5568, pregabalin, PTI 202, PTI 721, radiprodil, recombinant clostriadial neurotoxin protease, REGN 475, RPI 70, SAB 378, SCP 1, sufentanil/triazolam, SYN 116, tapentadol, thalidomide, URG 301, vanilloid receptor antagonists, zucapsaicin and topiramate.

In certain embodiments, the present invention provides for a method of treating Parkinson's disease, comprising administering one or more agents that increase BBB permeability in combination with one or more therapeutic agents. In some embodiments, such therapeutic agents mediate Parkinson's disease through the inhibition of dopa carboxylase, inhibition of COMT, inhibition of MAO-B, inhibition of alpha-synuclein, inhibition of LRRK2, inhibition of DJ-1. In other embodiments, Parkinson's disease is mediated by dopamine agonists or by delivery of Parkin or Pink1. Exemplary dopa carboxylase inhibitors include carbidopa, benserazide, cardidopa/levodopa, and benserazide/levodopa. Exemplary COMT inhibitors include tolcapone. Exemplary MAO-B inhibitors include selegine, rasagiline, amantadine, benzotropine, trihexyphenidyl, selegiline and entacapone. Exemplary dopamine agonists include bromocriptine, pergolide, pramipexole, ropinirole, piribedil, cabergoline, apomorphine and lisuride. In other embodiments, such therapeutic agents are selected from the group consisting of aplindore, apomorphine, autologous stem cell therapy, AV 201, AV 45, CERE 120, creatine, DAR 100, fipamezole, ioflupane 1231, MK 0657, NLX P101, nitisinone, preladenant, rotigotine, safinamide, SPN 803, SYN 115, traxoprodil, ubidecarenone, V 1512 and XP 21279.

In certain embodiments, the present invention provides for a method of treating Restless Leg syndrome, comprising administering one or more agents that increase BBB permeability in combination with one or more therapeutic agents. In some embodiments, such therapeutic agents are selected from a group consisting of ropinirole, pramipexole, rotigotine, opioids, aplindore, gabapentin enacarbil, pregabalin and benzodiazepines.

In certain embodiments, the present invention provides for a method of treating schizophrenia, comprising administering one or more agents that increase BBB permeability in combination with one or more therapeutic agents. In some embodiments, such therapeutic agents are selected from a group consisting of amisulpride, aripiprazole, asenapine, chlorpromazine, chlorprothixene, clozapine, droperidol, flupenthixol, fluphenazine, haloperidol, iloperidone, levomepromazine, mesoridazine, olanzapine, paliperidone, periciazine, perphenazine, pimozide, prochlorperazine, promazine, promethazine, quetiapine, risperidone, sertindole, thioridazine, thiothixene, trifluoperazine, triflupromazine, ziprasidone, zotepine, adipiplon, ADX 71149, armodafinil, ATI 9242, AVN 211, AZD 8529, BL 1020, cariprazine, CM 2395, davunetide, DCCCyB, EVP 6124, GSK 1144814, idazoxan, iloperidone, ITI 007, JNJ 17305600, lisdexamfetamine, loxapine, lurasidone, MEM 3454, MK 0249, NSA 789, ocaperidone, ORG 25935, paliperidone, PF 217830, PF 2545920, PF 3463275, pimavanserin, R 1678, sabcomeline, SB 773812, TGOFO2N, tiprolisant and zuclopenthixol. In another embodiment, schizophrenia is mediated by mGluR2 agonists.

In certain embodiments, the present invention provides for a method of treating a seizure disorder, comprising administering one or more agents that increase BBB permeability in combination with one or more therapeutic agents. In some embodiments, such therapeutic agents are selected from a group consisting of beclamide, brivaracetam, carbamazepine, carbatrol, clobazam, diastat, ethosuximide, felbamate, fosphenyloin, gabapentin, hydantoins, inovelon, lamotrigine, levetiracetam, mesuximide, neurotrin, nitrazepam, phenacemide, pheneturide, phenobarbitol, phenyloin, pregabalin, primidone, pyrimidindiones, vigabatrin, stiripentol, temazepam, tiagabine, topiramate, trileptal, valnoctamide, valproic acid, valproamide, lacosamide and zonisamide.

In certain embodiments, the present invention provides for a method of treating Tourette's syndrome, comprising administering one or more agents that increase BBB permeability in combination with one or more therapeutic agents. In some embodiments, such therapeutic agents are selected from a group consisting of typical antipsychotics, atypical psychotics, fluphenazine, haloperidol, pimozide, risperidonen ziprasidone and AFX 221.

In certain embodiments, the present invention provides for a method of treating a viral infection, comprising administering one or more agents that increase BBB permeability in combination with one or more therapeutic agents. In some embodiments, such therapeutic agents are selected from a group consisting of arginine butyrate, famiciclovir, tenofovir, zanamivir, oseltamivir, valomaciclovir and valacyclovir.

In certain embodiments, the present invention provides for a method of treating Wernicke-Korsakoff syndrome, comprising administering one or more agents that increase BBB permeability in combination with one or more therapeutic agents. In one embodiment, such therapeutic agent is thiamine.

In certain embodiments, the present invention provides for a method of treating stroke, comprising administering one or more agents that increase BBB permeability in combination with one or more therapeutic agents. Such therapeutic agents are selected from a group consisting of apixaban, aspirin/phosphatidylcholine, betrixaban, BVI 007, desmoteplase, MK 0724, MP 124, NA 1, NEU 2000, oxygenated fluorocarbin nutrient emulsion, rivaroxaban, SUN N8075, tenecteplase, traxoprodil, TS 011, V 10153 and zonampanel.

In certain embodiments, the present invention provides for a method of treating a personality disorder comprising administering one or more agents that increase BBB permeability in combination with one or more therapeutic agents. Such therapeutic agents are selected from a group consisting of ADX 71149, quetiapine and olanzapine.

In certain embodiments, the present invention provides for a method of treating a post-traumatic stress disorder, comprising administering one or more agents that increase BBB permeability in combination with one or more therapeutic agents. Such therapeutic agents are selected from a group consisting of cycloserine, MDMA, mirtazapine, nepicastat, topiramate and MK 0594.

In certain embodiments, the present invention provides for a method of treating a panic disorder, comprising administering one or more agents that increase BBB permeability in combination with one or more therapeutic agents. Such therapeutic agents are selected from a group consisting of cycloserine, escitalopram and ORG 25935.

In certain embodiments, the present invention provides for a method of providing neuroprotection, comprising administering one or more agents that increase BBB permeability in combination with one or more therapeutic agents. In one such embodiment, the therapeutic agent is epoetin alfa.

In certain embodiments, the present invention provides for a method of treating a neurological disorder, comprising administering one or more agents that increase BBB permeability in combination with one or more therapeutic agents. Such therapeutic agents are selected from a group consisting of A 0001, ABT 384, amantadine, KP 544, MK 5395, ORG 26041, ORG 50189, triacetyluridine and ubidecarenone.

In certain embodiments, the present invention provides for a method of treating a neurodegenerative disorder, comprising administering one or more agents that increase BBB permeability in combination with one or more therapeutic agents. Such therapeutic agents are AV 133 and OSI 754.

In certain embodiments, the present invention provides for a method of treating a female sexual dysfunction, comprising administering one or more agents that increase BBB permeability in combination with one or more therapeutic agents. Such therapeutic agents are selected from a group consisting of bremelanotide, flibanserin and testosterone.

In certain embodiments, the present invention provides for a method of treating a cognition disorder, comprising administering one or more agents that increase BBB permeability in combination with one or more therapeutic agents. Such therapeutic agents are selected from a group consisting of ABT 560, AV 965, DAR 100, HTC 867, IPL 455903, levafetamine, LU AE 58054, nefiracetam, PF 3654746, PF 4447943, phenserine, PRX 07034, R-phenserine, SYN 114 and SYN 120.

In certain embodiments, the present invention provides for a method of treating a CNS disorder, comprising administering one or more agents that increase BBB permeability in combination with one or more therapeutic agents. Such therapeutic agents are selected from a group consisting of ecopipam, isovaleramide, pivagabine esters, GSK 249320 and ALKS 33.

In certain embodiments, the present invention provides for a method of treating a dementia, comprising administering one or more agents that increase BBB permeability in combination with one or more therapeutic agents. Such therapeutic agents are selected from a group consisting of ABT 560, ADS 8703 and nefiracetam.

In certain embodiments, the present invention provides for a method of treating Asperger's syndrome, comprising administering one or more agents that increase BBB permeability in combination with one or more therapeutic agents. One such therapeutic agent is aripiprazole.

In certain embodiments, the present invention provides for a method of treating a autoimmune disorder, comprising administering one or more agents that increase BBB permeability in combination with one or more therapeutic agents. Such therapeutic agents are selected from a group consisting of PRTX 100, semapimod, SGN 70 and VBY 129.

Exemplary disorders and/or conditions suitable for treatment in accordance with the present invention include acquired epileptiform aphasia, acute disseminated encephalomyelitis, adrenoleukodystrophy, agenesis of the corpus callosum, agnosia, aicardi syndrome, Alexander disease, Alpers' disease, alternating hemiplegia, Alzheimer's disease, amyotrophic lateral sclerosis, anencephaly, Angelman syndrome, angiomatosis, anoxia, aphasia, apraxia, arachnoid cysts, arachnoiditis, Arnold-chiari malformation, arteriovenous malformation, Asperger's syndrome, ataxia telangiectasia, attention deficit hyperactivity disorder, autism, auditory processing disorder, autonomic dysfunction, back pain, Batten disease, Behcet's disease, Bell's palsy, benign essential blepharospasm, benign focal amyotrophy, benign intracranial hypertension, bilateral frontoparietal polymicrogyria, binswanger's disease, blepharospasm, Bloch-sulzberger syndrome, brachial plexus injury, brain abscess, brain damage, brain injury, brain tumor, spinal tumor, Brown-sequard syndrome, canavan disease, carpal tunnel syndrome (cts), causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, charcot-marie-tooth disease, chiari malformation, chorea, chronic inflammatory demyelinating polyneuropathy (“CIDP”), chronic pain, chronic regional pain syndrome, Coffin lowry syndrome, coma (including persistent vegetative state), congenital facial diplegia, corticobasal degeneration, cranial arteritis, craniosynostosis, Creutzfeldt Jakobdisease, cumulative trauma disorders, Cushing's syndrome, cytomegalic inclusion body disease (“CIBD”), cytomegalovirus infection, dandy-walker syndrome, Dawson disease, de morsier's syndrome, Dejerine-klumpke palsy, Dejerine-sottas disease, delayed sleep phase syndrome, dementia, dermatomyositis, developmental dyspraxia, diabetic neuropathy, diffuse sclerosis, dysautonomia, dyscalculia, dysgraphia, dyslexia, dystonia, early infantile epileptic encephalopathy, empty sella syndrome, encephalitis, encephalocele, encephalotrigeminal angiomatosis, encopresis, epilepsy, Erb's palsy, erythromelalgia, essential tremor, Fabry's disease, Fahr's syndrome, fainting, familial spastic paralysis, febrile seizures, fisher syndrome, Friedreich's ataxia, Gaucher's disease, Gerstmann's syndrome, giant cell arteritis, giant cell inclusion disease, globoid cell leukodystrophy, gray matter heterotopia, Guillain-bane syndrome, htiv-1 associated myelopathy, Hallervorden-spatz disease, head injury, headache, hemifacial spasm, hereditary spastic paraplegia, heredopathia atactica polyneuritiformis, herpes zoster oticus, herpes zoster, hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile refsum disease, infantile spasms, inflammatory myopathy, intracranial cyst, intracranial hypertension, Joubert syndrome, Kearns-sayre syndrome, Kennedy disease, kinsbourne syndrome, Klippel feil syndrome, Krabbe disease, Kugelberg-welander disease, kuru, lafora disease, Lambert-eaton myasthenic syndrome, Landau-kleffner syndrome, lateral medullary (Wallenberg) syndrome, learning disabilities, leigh's disease, Lennox-gastaut syndrome, Lesch-nyhan syndrome, leukodystrophy, lewy body dementia, lissencephaly, locked-in syndrome, Lou Gehrig's disease, lumbar disc disease, lyme disease—neurological sequelae, machado-joseph disease (spinocerebellar ataxia type 3), macrencephaly, megalencephaly, Melkersson-rosenthal syndrome, Meniere's disease, meningitis, Menkes disease, metachromatic leukodystrophy, microcephaly, migraine, Miller Fisher syndrome, mini-strokes, mitochondrial myopathies, mobius syndrome, monomelic amyotrophy, motor neurone disease, motor skills disorder, moyamoya disease, mucopolysaccharidoses, multi-infarct dementia, multifocal motor neuropathy, multiple sclerosis, multiple system atrophy with postural hypotension, muscular dystrophy, myalgic encephalomyelitis, myasthenia gravis, myelinoclastic diffuse sclerosis, myoclonic encephalopathy of infants, myoclonus, myopathy, myotubular myopathy, myotonia congenita, narcolepsy, neurofibromatosis, neuroleptic malignant syndrome, neurological manifestations of aids, neurological sequelae of lupus, neuromyotonia, neuronal ceroid lipofuscinosis, neuronal migration disorders, niemann-pick disease, non 24-hour sleep-wake syndrome, nonverbal learning disorder, O'sullivan-mcleod syndrome, occipital neuralgia, occult spinal dysraphism sequence, ohtahara syndrome, olivopontocerebellar atrophy, opsoclonus myoclonus syndrome, optic neuritis, orthostatic hypotension, overuse syndrome, palinopsia, paresthesia, Parkinson's disease, paramyotonia congenita, paraneoplastic diseases, paroxysmal attacks, parry-romberg syndrome (also known as rombergs syndrome), pelizaeus-merzbacher disease, periodic paralyses, peripheral neuropathy, persistent vegetative state, pervasive developmental disorders, photic sneeze reflex, phytanic acid storage disease, pick's disease, pinched nerve, pituitary tumors, pmg, polio, polymicrogyria, polymyositis, porencephaly, post-polio syndrome, postherpetic neuralgia (“PHN”), postinfectious encephalomyelitis, postural hypotension, Prader-willi syndrome, primary lateral sclerosis, prion diseases, progressive hemifacial atrophy (also known as Romberg's syndrome), progressive multifocal leukoencephalopathy, progressive sclerosing poliodystrophy, progressive supranuclear palsy, pseudotumor cerebri, ramsay-hunt syndrome (type I and type II), Rasmussen's encephalitis, reflex sympathetic dystrophy syndrome, refsum disease, repetitive motion disorders, repetitive stress injury, restless legs syndrome, retrovirus-associated myelopathy, rett syndrome, Reye's syndrome, Romberg's syndrome, rabies, Saint Vitus' dance, Sandhoff disease, schizophrenia, Schilder's disease, schizencephaly, sensory integration dysfunction, septo-optic dysplasia, shaken baby syndrome, shingles, Shy-drager syndrome, Sjögren's syndrome, sleep apnea, sleeping sickness, snatiation, Sotos syndrome, spasticity, spina bifida, spinal cord injury, spinal cord tumors, spinal muscular atrophy, spinal stenosis, Steele-richardson-olszewski syndrome, see progressive supranuclear palsy, spinocerebellar ataxia, stiff-person syndrome, stroke, Sturge-weber syndrome, subacute sclerosing panencephalitis, subcortical arteriosclerotic encephalopathy, superficial siderosis, sydenham's chorea, syncope, synesthesia, syringomyelia, tardive dyskinesia, Tay-sachs disease, temporal arteritis, tetanus, tethered spinal cord syndrome, Thomsen disease, thoracic outlet syndrome, tic douloureux, Todd's paralysis, Tourette syndrome, transient ischemic attack, transmissible spongiform encephalopathies, transverse myelitis, traumatic brain injury, tremor, trigeminal neuralgia, tropical spastic paraparesis, trypanosomiasis, tuberous sclerosis, vasculitis including temporal arteritis, Von Hippel-lindau disease (“VHL”), Viliuisk encephalomyelitis (“VE”), Wallenberg's syndrome, Werdnig-hoffman disease, west syndrome, whiplash, Williams syndrome, Wilson's disease, and Zellweger syndrome. It is thus appreciated that all CNS-related states and disorders could be treated through the BBB route of drug delivery.

Another aspect of the present invention relates to a method of delivering a macromolecule therapeutic agent to the brain of a subject. This method involves administering to the subject (a) an agent which activates both of A₁ and A_{2A} adenosine receptors and (b) the macromolecular therapeutic.

In certain embodiments, the macromolecular therapeutic agent may be a bioactive protein or peptide agent. Examples of such bioactive protein or peptides include a cell modulating peptide, a chemotactic peptide, an anticoagulant peptide, an antithrombotic peptide, an anti-tumor peptide, an anti-infectious peptide, a growth potentiating peptide, and an anti-inflammatory peptide. Examples of proteins include antibodies, enzymes, steroids, growth hormone and growth hormone-releasing hormone, gonadotropin-releasing hormone and its agonist and antagonist analogues, somatostatin and its analogues, gonadotropins, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. In some aspects of the invention, the BBB permeability is modulated by one or more methods herein above to deliver an antibiotic, or an anti-infectious therapeutic capable agent. Such anti-infectious agents reduce the activity of or kills a microorganism.

The nature of the peptide agent is not limited, other than comprising amino acid residues. The peptide agent can be a synthetic or a naturally occurring peptide, including a variant or derivative of a naturally occurring peptide. The peptide can be a linear peptide, cyclic peptide, constrained peptide, or a peptidomimetic. Methods for making cyclic peptides are well known in the art. For example, cyclization can be achieved in a head-to-tail manner, side chain to the N- or C-terminus residues, as well as cyclizations using linkers. The selectivity and activity of the cyclic peptide depends on the overall ring size of the cyclic peptide which controls its three dimensional structure. Cyclization thus provides a powerful tool for probing progression of disease states, as well as targeting specific self-aggregation states of diseased proteins.

In some embodiments, the peptide agent specifically binds to a target protein or structure associated with a neurological condition. In accordance with these embodiments, the invention provides agents useful for the selective targeting of a target protein or structure associated with a neurological condition, for diagnosis or therapy. Peptide agents useful in accordance with the present invention are described in, for example, U.S. Patent Application Publication 2009/0238754 to Wegrzyn et al., which is hereby incorporated by reference in its entirety.

In other embodiments, the peptide agent specifically binds to a target protein or structure associated with other neurological conditions, such as stroke, cerebrovascular disease, epilepsy, transmissible spongiform encephalopathy (TSE); Aβ-peptide in amyloid plaques of Alzheimer's disease (Aβ), cerebral amyloid angiopathy (CAA), and cerebral vascular disease (CVD); α-synuclein deposits in Lewy bodies of Parkinson's disease, tau in neurofibrillary tangles in frontal temporal dementia and Pick's disease; superoxide dismutase in amylotrophic lateral sclerosis; and Huntingtin in Huntington's disease and benign and cancerous brain tumors such as glioblastoma's, pituitary tumors, or meningiomas.

In some embodiments, the peptide agent undergoes a conformational shift other than the alpha-helical to beta-sheet shift discussed above, such as a beta-sheet to alpha-helical shift, an unstructured to beta-sheet shift, etc. Such peptide agents may undergo such conformational shifts upon interaction with target peptides or structures associated with a neurological condition.

In other embodiments, the peptide agent is an antibody that specifically binds to a target protein or structure associated with a neurological condition, such as a target protein or structure (such as a specific conformation or state of self-aggregation) associated with an amyloidogenic disease, such as the anti-amyloid antibody 6E10, and NG8. Other anti-amyloid antibodies are known in the art, as are antibodies that specifically bind to proteins or structures associated with other neurological conditions.

In certain embodiments, the macromolecular therapeutic agent is a monoclonal antibody. Suitable monoclonal antibodies include 6E10, PF-04360365, 131I-chTNT-1/B MAb, 131I-L19SIP, 177Lu-J591, ABT-874, AIN457, alemtuzumab, anti-PDGFR alpha monoclonal antibody IMC-3G3, astatine At 211 monoclonal antibody 8106, Bapineuzumab, Bevacizumab, cetuximab, cixutumumab, Daclizumab, Hu MiK-beta-1, HuMax-EGFr, iodine I 131 monoclonal antibody 3F8, iodine I 131 monoclonal antibody 8106, iodine I 131 monoclonal antibody 8H9, iodine I 131 monoclonal antibody TNT-1/B, LMB-7 immunotoxin, MAb-425, MGAWN1, Me1-14 F(ab′)2, M-T412, Natalizumab, Neuradiab, Nimotuzumab, Ofatumumab, Panitumumab, Ramucirumab, ranibizumab, SDZ MSL-109, Solanezumab, Trastuzumab, Ustekinumab, Zalutumumab, Tanezumab, Aflibercept, MEDI-578, REGN475, Muromonab-CD3, Abiximab, Rituximab, Basiliximab, Palivizumab, Infliximab, Gemtuzumab ozogamicin, Ibritumomab tiuxetan, Adalimumab, Omalizumab, Tositumomab, Tositumomab-I131, Efalizumab, Abciximab, Certolizumab pegol, Eculizumab, AMG-162, Zanolimumab, MDX-010, Anti0MRSA mAb, Pexelizumab, Mepolizumab, Epratuzumab, Anti-RSV mAb, Afelimomab, Catumaxomab, WX-G250, or combinations thereof.

In certain embodiments, the macromolecular therapeutic agent is a peptide detection agent. For example, peptide detection agents include fluorescent proteins, such as Green Flourescent Protein (GFP), streptavidin, enzymes, enzyme substrates, and other peptide detection agents known in the art.

In other embodiments, the macromolecular therapeutic agent includes peptide macromolecules and small peptides. For example, neurotrophic proteins are useful as peptide agents in the context of the methods described herein. Neurotrophic proteins include nerve growth factor (NGF), brain-derived neurotrophic factor (BDNF), neurotrophin-3 (NT-3), neurotrophin-4 (NT-4), neurotrophin-5 (NT-5), insulin-like growth factors (IGF-I and IGF-II), glial cell line derived neurotrophic factor (GDNF), fibroblast growth factor (FGF), ciliary neurotrophic factor (CNTF), epidermal growth factor (EGF), glia-derived nexin (GDN), transforming growth factor (TGF-α and TGF-β), interleukin, platelet-derived growth factor (PDGF) and S100β protein, as well as bioactive derivatives and analogues thereof.

Neuroactive peptides also include the subclasses of hypothalamic-releasing hormones, neurohypophyseal hormones, pituitary peptides, invertebrate peptides, gastrointestinal peptides, those peptides found in the heart, such as atrial naturetic peptide, and other neuroactive peptides. Hypothalamic releasing hormones include, for example, thyrotropin-releasing hormones, gonadotropin-releasing hormone, somatostatins, corticotropin-releasing hormone and growth hormone-releasing hormone. Neurohypophyseal hormones include, for example, compounds such as vasopressin, oxytocin, and neurophysins. Pituitary peptides include, for example, adrenocorticotropic hormone, β-endorphin, α-melanocyte-stimulating hormone, prolactin, luteinizing hormone, growth hormone, and thyrotropin. Suitable invertebrate peptides include, for example, FMRF amide, hydra head activator, proctolin, small cardiac peptides, myomodulins, buccolins, egg-laying hormone and bag cell peptides. Gastrointestinal peptides include, for example, vasoactive intestinal peptide, cholecystokinin, gastrin, neurotensin, methionineenkephalin, leucine-enkephalin, insulin and insulin-like growth factors I and II, glucagon, peptide histidine isoleucineamide, bombesin, motilin and secretins. Examples of other neuroactive peptides include angiotensin II, bradykinin, dynorphin, opiocortins, sleep peptide(s), calcitonin, CGRP (calcitonin gene-related peptide), neuropeptide Y, neuropeptide Yy, galanin, substance K (neurokinin), physalaemin, Kassinin, uperolein, eledoisin and atrial naturetic peptide.

In yet further embodiments, the macromolecular therapeutic agent is a protein associated with membranes of synaptic vesicles, such as calcium-binding proteins and other synaptic vesicle proteins. The subclass of calcium-binding proteins includes the cytoskeleton-associated proteins, such as caldesmon, annexins, calelectrin (mammalian), calelectrin (torpedo), calpactin I, calpactin complex, calpactin II, endonexin I, endonexin II, protein II, synexin I; and enzyme modulators, such as p65. Other synaptic vesicle proteins include inhibitors of mobilization (such as synapsin Ia,b and synapsin IIa,b), possible fusion proteins such as synaptophysin, and proteins of unknown function such as p29, VAMP-1,2 (synaptobrevin), VAT1, rab 3A, and rab 3B.

Macromolecular therapeutic agents also include α-, β- and γ-interferon, epoetin, Fligrastim, Sargramostin, CSF-GM, human-IL, TNF and other biotechnology drugs.

Macromolecular therapeutic agents also include peptides, proteins and antibodies obtained using recombinant biotechnology methods.

Macromolecular therapeutic agents also include “anti-amyloid agents” or “anti-amyloidogenic agents,” which directly or indirectly inhibit proteins from aggregating and/or forming amyloid plaques or deposits and/or promotes disaggregation or reduction of amyloid plaques or deposits. Anti-amyloid agents also include agents generally referred to in the art as “amyloid busters” or “plaque busters.” These include drugs which are peptidomimetic and interact with amyloid fibrils to slowly dissolve them. “Peptidomimetic” means that a biomolecule mimics the activity of another biologically active peptide molecule. “Amyloid busters” or “plaque busters” also include agents which absorb co-factors necessary for the amyloid fibrils to remain stable.

Anti-amyloid agents include antibodies and peptide probes, as described in PCT application PCT/US2007/016738 (WO 2008/013859) and U.S. patent application Ser. No. 11/828,953, the entire contents of which are incorporated herein by reference in their entirety. As described therein, a peptide probe for a given target protein specifically binds to that protein, and may preferentially bind to a specific structural form of the target protein. While not wanting to be bound by any theory, it is believed that binding of target protein by a peptide probe will prevent the formation of higher order assemblies of the target protein, thereby preventing or treating the disease associated with the target protein, and/or preventing further progression of the disease. For example, binding of a peptide probe to a monomer of the target protein will prevent self-aggregation of the target protein. Similarly, binding of a peptide probe to a soluble oligomer or an insoluble aggregate will prevent further aggregation and protofibril and fibril formation, while binding of a peptide probe to a protofibril or fibril will prevent further extension of that structure. In addition to blocking further aggregation, this binding also may shift the equilibrium back to a state more favorable to soluble monomers, further halting the progression of the disease and alleviating disease symptoms.

In another embodiment, the macromolecular therapeutic agent is about 150 kDa in size. In yet another embodiment, the therapeutic is up to about 10,000 Da in size, up to about 70,000 Da in size, or up to about 150 kDa in size. In still further embodiments the therapeutic is between about 10,000 and about 70,000 Da, between about 70,000 Da and 150 kDa, or between about 10,000 Da and about 150 kDa in size.

In some embodiments, a macromolecular therapeutic agent is a polynucleotide.

In certain embodiments, a polynucleotide is a plasmid DNA (pDNA). As used herein, pDNA is defined as a circular, double-stranded DNA that contains a DNA sequence (cDNA or complementary DNA) that is to be expressed in cells either in culture or in vivo. The size of pDNA can range from 3 kilo base pairs (kb) to greater than 50 kb. The cDNA that is contained within plasmid DNA is usually between 1-5 kb in length, but can be greater if larger genes are incorporated. pDNA may also incorporate other sequences that allow it to be properly and efficiently expressed in mammalian cells, as well as replicated in bacterial cells. In some embodiments, pDNA expresses a therapeutic gene in cell culture, animals, or humans that possess a defective or missing gene that is responsible for disease.

In some embodiments, a polynucleotide is capable of silencing gene expression via RNA interference (RNAi). As defined herein, RNAi is a cellular mechanism that suppresses gene expression during translation and/or hinders the transcription of genes through destruction of messenger RNA (mRNA). Without wishing to be bound by any particular theory, it is believed that endogenous double-stranded RNA located in the cell are processed into 20-25 nt short-interfering RNA (siRNA) by the enzyme Dicer. siRNA subsequently binds to the RISC complex (RNA-induced silencing nuclease complex), and the guide strand of the siRNA anneals to the target mRNA. The nuclease activity of the RISC complex then cleaves the mRNA, which is subsequently degraded (Nat. Rev. Mol. Cell. Biol., 2007, 8, 23).

In some embodiments, a polynucleotide is a siRNA. As used herein, siRNA is defined as a linear, double-stranded RNA that is 20-25 nucleotides (nt) in length and possesses a 2 nt, 3′ overhang on each end which can induce gene knockdown in cell culture or in vivo via RNAi. In some embodiments of the invention, an siRNA suppresses disease-relevant gene expression in cell culture, animals, or humans.

In some embodiments, a polynucleotide is pDNA that expresses a short-hairpin RNA (shRNA). As used herein, shRNA is a linear, double-stranded RNA, possessing a tight hairpin turn, which is synthesized in cells through transfection and expression of a exogenous pDNA. Without wishing to be bound by any particular theory, it is believed that the shRNA hairpin structure is cleaved to produce siRNA, which mediates gene silencing via RNA interference. In some embodiments of the invention, a shRNA suppresses gene expression in cell culture, animals, or humans that are responsible for a disease via RNAi.

In some embodiments, a polynucleotide is a microRNA (miRNA). As used herein, miRNA is a linear, single-stranded RNA that ranges between 21-23 nt in length and regulates gene expression via RNAi (Cell, 2004, 116, 281). In some embodiments, an miRNA suppresses gene expression in cell culture, animals, or humans that are responsible for a disease via RNAi.

In some embodiments, a polynucleotide is a messenger RNA (mRNA). As used herein, mRNA is defined as a linear, single stranded RNA molecule, which is responsible for translation of genes (from DNA) into proteins. In some embodiments, an mRNA is encoded from a plasmid cDNA to serve as the template for protein translation. In some embodiments, an mRNA translates therapeutic proteins, in vitro and/or in vivo, which can treat disease.

In some embodiments of the invention, a polynucleotide is an antisense RNA (aRNA). As used herein, aRNA is a linear, single-stranded RNA that is complementary to a targeted mRNA located in a cell. Without wishing to be bound by any particular theory, it is believed that aRNA inhibits translation of a complementary mRNA by pairing with it and obstructing the cellular translation machinery. It is believed that the mechanism of action for aRNA is different from RNAi because the paired mRNA is not destroyed. In some embodiments, an aRNA suppresses gene expression in cell culture, animals, or humans that are responsible for a disease by binding mRNA and physically obstructing translation.

In one embodiment, the agent that activates both of the A₁ and A_{2A} adenosine receptors is administered before the therapeutic macromolecule. In further embodiments, the agent that activates both of the A₁ and A_{2A} adenosine receptors may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic macromolecule agent.

In another embodiment, the agent or agents that activate both of the A₁ and A_{2A} adenosine receptors is administered simultaneously with the therapeutic agent or therapeutic macromolecule.

Another aspect of the present invention relates to a method for treating a CNS disease, disorder, or condition in a subject. This method involves administering to the subject at least one agent which activates both of the A₁ and the A_{2A} adenosine receptors and a therapeutic agent.

Another aspect of the present invention relates to a method of temporarily increasing the permeability of the blood brain barrier of a subject. This method includes selecting a subject in need of a temporary increase in permeability of the blood brain barrier, providing an agent which activates either the A₁ or the A_{2A} adenosine receptor, and administering to the selected subject either the A₁ or the A_{2A} adenosine receptor activating agent under conditions effective to temporarily increase the permeability of the blood brain barrier.

In one embodiment, the agent that activates the A₁ or the A_{2A} adenosine receptor is administered before the therapeutic agent. In further embodiments, the agent that activates the A₁ or the A_{2A} adenosine receptor may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent.

Yet another aspect of the present invention relates to a method of remodeling an actin cytoskeleton of a BBB endothelial cell. This method involves contacting an endothelial cell with one or more agents that activates both of the A₁ and the A_{2A} adenosine receptors.

The actin cytoskeleton is vital for the maintenance of cell shape. Endothelial barrier permeability can be affected by reorganization of the actin cytoskeleton. The actin cytoskeleton is organized into three distinct structures: the cortical actin rim, actomyosin stress fibers, and actin cross-linking of the membrane skeleton (Prasain et al., “The Actin Cytoskeleton in Endothelial Cell Phenotypes,” Microvasc. Res. 77:53-63 (2009), which is hereby incorporated by reference in its entirety). These structures have unique roles in controlling endothelial cell shape.

According to one embodiment, the actin cytoskeleton remodeling increases space between endothelial cells and increases BBB permeability.

Suitable A₁ and A_{2A} adenosine receptor activators are disclosed above.

In one embodiment according to this aspect of the present invention, the activation of both of the A₁ and A_{2A} adenosine receptors is synergistic with respect to BBB permeability. In yet another embodiment, the activation of both of the A₁ and A_{2A} adenosine receptors is additive with respect to BBB permeability.

### EXEMPLIFICATION

Assaying a provided combination therapy, or composition thereof, can be performed using methods known in the art. Such methods include those described in Advanced Drug Delivery Rev's 36(2-3): 165-179 (April 1999); Pharm. and Exp. Ther. 303(3): 928-936 (December 2002); and J. Drug Target June 2001; 9(3): 209-21.

An exemplary in vivo model is performed in the following manner. Toxicity studies are routinely conducted to determine a nondebilitating, sublethal dose of the test compound. This dose is coadministered via the mouse tail vein along with a tracer cocktail containing 3H-sucrose and 1251-BSA. The animals are perfused 15 or 60 minutes later, and the brains are removed, weighed, and assayed by liquid scintillation spectrometry. The disintegrations per minute per wet weight is averaged for a minimum of four animals per experimental group. Standard error of the mean (SEM) values are less than 10 percent. The between-experiment error for the control groups is also less than 10 percent. The fold increase in tracer content is calculated by dividing the average treated DPM by that of the control DPM. Compounds that cause an increase of 1.5 fold or more are considered candidates for testing in the behavioral assays. These assays are designed to demonstrate delivery of a drug into the brain parenchyma at levels sufficient to have a therapeutic effect. Morphine and the naturally occurring peptides, endorphin and enkephalin, bind to μ-opioid receptors in the brain and suppress the sensation of pain. This analgesic effect can be demonstrated with mice in the hot-plate assay. Mice are placed on a surface uniformly heated to 55° C. The time it takes for the mouse to respond to the heat stimulus by licking its paws is measured. Morphine (MW 700) delivered intravenously at doses of 1 to 10 mg/kg has an analgesic effect in that it increases the latency of response to the heat stimulus measured 15 minutes after injection. The latency is expressed as percentanalgesia. The purpose of these experiments is to test the ability of putative BBB openers to shift the morphine dose-analgesic response curve in the leftward direction, Indicating enhanced delivery of morphine to the brain parenchyma as reflected by increased paw-lick latency time. Similar experiments are conducted using the less permeant but significantly more costly enkephalin.

### Example 1

### Mice

Cd73^{-/-} mice have been previously described (Thompson et al., “Crucial Role for Ecto-5′-Nucleotidase (CD73) in Vascular Leakage During Hypoxia,” J. Exp. Med. 200:1395-1405 (2004), which is hereby incorporated by reference in its entirety) and have been backcrossed to C57BL/6 for 14 generations. Cd73 mice have no overt susceptibility to infection and appear normal based on the size and cellular composition of their lymphoid organs and their T and B cell responses in in vivo and in vitro assays (Thompson et al., “Crucial Role for Ecto-5′-Nucleotidase (CD73) in Vascular Leakage During Hypoxia,” J. Exp. Med. 200:1395-1405 (2004), which is hereby incorporated by reference in its entirety). C57BL/6 and tcrα^{-/-} mice on the C57BL/6 background were purchased from The Jackson Laboratories. Mice were bred and housed under specific pathogen-free conditions at Cornell University or the University of Turku. For adenosine receptor blockade experiments, mice were given drinking water supplemented with 0.6 g/L of caffeine (Sigma) or 2 mg/kg SCH58261 (1 mg/kg s.c. and 1 mg/kg i.p.) in DMSO (45% vol. in PBS) or 45% DMSO alone starting 1 day before EAE induction and continuing throughout the experiment. All procedures performed on mice were approved by the relevant animal review committee.

### Example 2

### EAE Induction and Scoring

EAE was induced by subjecting mice to the myelin oligodendrocyte glycoprotein (“MOG”) EAE-inducing regimen as described in Swanborg, “Experimental Autoimmune Encephalomyelitis in Rodents as a Model for Human Demyelinating Disease,” Clin. Immunol. Immunopathol. 77:4-13 (1995) and Bynoe et al., “Epicutaneous Immunization with Autoantigenic Peptides Induces T Suppressor Cells that Prevent Experimental Allergic Encephalomyelitis,” Immunity 19:317-328 (2003), which are hereby incorporated by reference in their entirety. Briefly, a 1:1 emulsion of MOG₃₅₋₅₅ peptide (3 mg/ml in PBS) (Invitrogen) and complete Freund's adjuvant (CFA, Sigma) was injected subcutaneously (50 μl) into each flank. Pertussis toxin (PTX, 20 ng) (Biological Laboratories Inc.) was given intravenously (200 μl in PBS) at the time of immunization and again 2 days later. Mice were scored daily for EAE based on disease symptom severity; 0=no disease, 0.5-1=weak/limp tail, 2=limp tail and partial hind limb paralysis, 3=total hind limb paralysis, 4=both hind limb and fore limb paralysis, 5=death. Mice with a score of 4 were euthanized.

### Example 3

### T Cell Preparations and Adoptive Transfer

Mice were primed with MOG₃₅₋₅₅ peptide in CFA without PTX. After one week, lymphocytes were harvested from spleen and lymph nodes and incubated with ACK buffer (0.15M NH₄Cl, 1 mM KHCO₃, 0.1 mM EDTA, pH 7.3) to lyse red blood cells. Cells were incubated with antibodies to CD8 (TIB-105), IA^{b,d,v,p,q,r} (212.A1), FcR (2.4-G2), B220 (TIB-164), NK1.1 (HB191) and then BioMag goat anti-mouse IgG, IgM, and goat anti-rat IgG (Qiagen). After negative magnetic enrichment, CD4 cells were used either directly or further sorted into specific subpopulations. For sorting, cells were stained with antibodies to CD4 (RM4-5) and CD73 (TY/23), and in some experiments CD25 (PC61), and then isolated utilizing a FACSAria (BD Biosciences). Post-sort purity was routinely >99%. For adoptive transfer, total CD4⁺ or sorted T cells were washed and resuspended in sterile PBS. Recipient mice received ≦2.5×10⁶ cells i.v. in 200 μl of sterile PBS.

### Example 4

### Flow Cytometry

Cell suspensions were stained with fluorochrome-conjugated antibodies for CD4 (RM4-5), CD73 (TY/23), or FoxP3 (FJK-16s). Intracellular FoxP3 staining was carried out according to the manufacturer's instructions (eBioscience). Stained cells were acquired on a FACSCalibur (BD Biosciences). Data were analyzed with FlowJo software (Tree Star).

### Example 5

### T cell Cytokine Assay

Sorted T cells from MOG-immunized mice were cultured in the presence of irradiated C57BL/6 splenocytes with 0 or 10 μM MOG peptide. Supernatants were collected at 18 hrs and analyzed utilizing the Bio-plex cytokine (Biorad) assay for IL-2, IL-4, IL-5, IL-10, IL-13, IL-17, IL-1β, and TNFα.

### Example 6

### Immunohistochemistry (“IHC”)

Anesthetized mice were perfused with PBS, and brains, spleens, and spinal cords were isolated and snap frozen in Tissue Tek-OCT medium. Five μm sections (brains in a sagittal orientation) were affixed to Supefrost/Plus slides (Fisher), fixed in acetone, and stored at −80° C. For immunostaining, slides were thawed and treated with 0.03% H₂O₂ in PBS to block endogenous peroxidase, blocked with Casein (Vector) in normal goat serum (Zymed), and then incubated with anti-CD45 (YW62.3), anti-CD4 (RM4-5), or anti-ICAM-1 (3E2). Slides were incubated with biotinylated goat anti-rat Ig (Jackson ImmunoResearch) and streptavidin-HRP (Zymed) and developed with an AEC (Red) substrate kit (Zymed) and a hematoxylin counterstain. Cover slips were mounted with Fluoromount-G and photographed under light (Zeiss).

### Example 7

### Real Time q-PCR

Using Trizol (Invitrogen), RNA was isolated from the Z310 choroid plexus cell line (Zheng et al., “Establishment and Characterization of an Immortalized Z310 Choroidal Epithelial Cell Line from Murine Choroid Plexus,” Brain Res. 958:371-380 (2002), which is hereby incorporated by reference in its entirety). cDNA was synthesized using a Reverse-iT kit (ABGene). Primers (available upon request) specific for ARs were used to determine gene expression levels and standardized to the GAPDH housekeeping gene levels using a SYBR-Green kit (ABGene) run on an ABI 7500 real time PCR system. Melt curve analyses were performed to measure the specificity for each qPCR product.

### Example 8

### Evaluation of the Role of CD73 in EAE

Due to the immunomodulatory and immunosuppressive properties of adenosine, the role of CD73 in EAE was evaluated. Based on a report of exacerbated EAE in A1 adenosine receptor (AR)-deficient mice (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety), cd73^{-/-} mice that are unable to catalyze the production of extracellular adenosine were expected to experience severe EAE. Surprisingly, cd73^{-/-} mice were highly resistant to the induction of EAE. However, CD4⁺ T cells from cd73^{-/-} mice do possess the capacity to generate an immune response against CNS antigens and cause severe EAE when adoptively transferred into cd73^{+/+} T cell-deficient mice. CD73⁺CD4⁺ T cells from wild type mice also caused disease when transferred into cd73^{-/-} recipients, suggesting that CD73 expression, either on lymphocytes or in the CNS, is required for lymphocyte entry into the brain during EAE. Since cd73^{+/+} mice were protected from EAE induction by the broad spectrum AR antagonist caffeine and the A_{2A} AR specific antagonist SCH58261, this data suggests that the extracellular adenosine generated by CD73, and not CD73 itself, regulates the entry of lymphocytes into the CNS during EAE. These results are the first to demonstrate a role for CD73 and adenosine in regulating the development of EAE.

### Example 9

### Cd73^{-/-} Mice are Resistant to EAE Induction

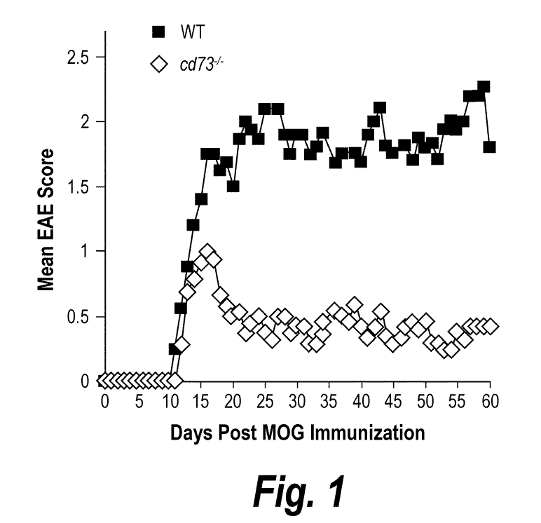

To determine if CD73 plays a role in controlling inflammation during EAE progression, cd73^{-/-} and wild type (cd73^{+/+}) mice were subjected to the myelin oligodendrocyte glycoprotein (“MOG”) EAE-inducing regimen (Swanborg, “Experimental Autoimmune Encephalomyelitis in Rodents as a Model for Human Demyelinating Disease,” Clin. Immunol. Immunopathol. 77:4-13 (1995); Bynoe et al., “Epicutaneous Immunization with Autoantigenic Peptides Induces T Suppressor Cells that Prevent Experimental Allergic Encephalomyelitis,” Immunity 19:317-328 (2003), which are hereby incorporated by reference in their entirety). Daily monitoring for EAE development revealed that cd73^{-/-} mice consistently displayed dramatically reduced disease severity compared to their wild type counterparts (FIG. 1). By three weeks after disease induction, cd73^{-/-} mice had an average EAE score of only 0.5 (weak tail) compared to 2.0 (limp tail and partial hind limb paralysis) for wild type mice (FIG. 1).

### Example 10

### CD4⁺ T Cells From cd73^{-/-} Mice Respond to MOG Immunization

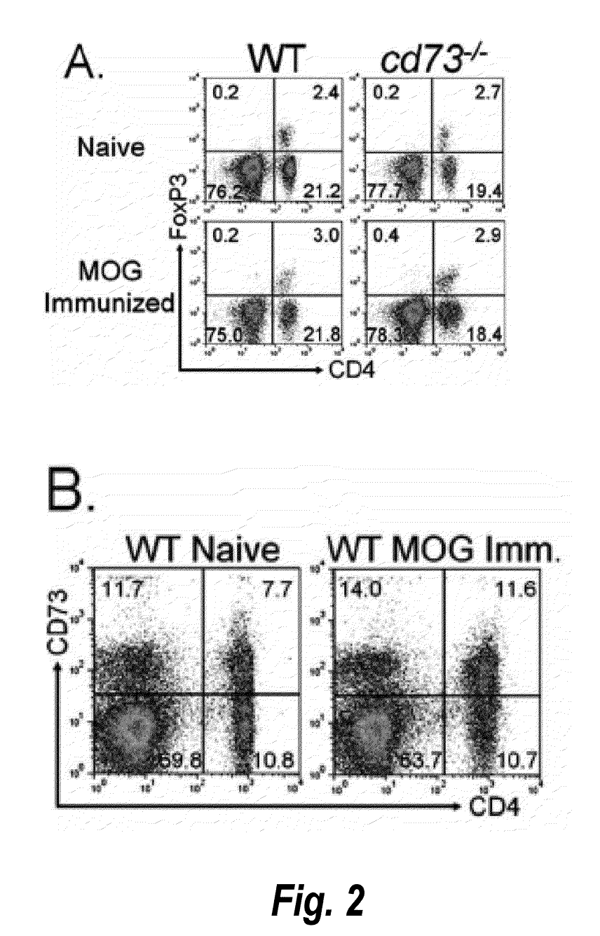

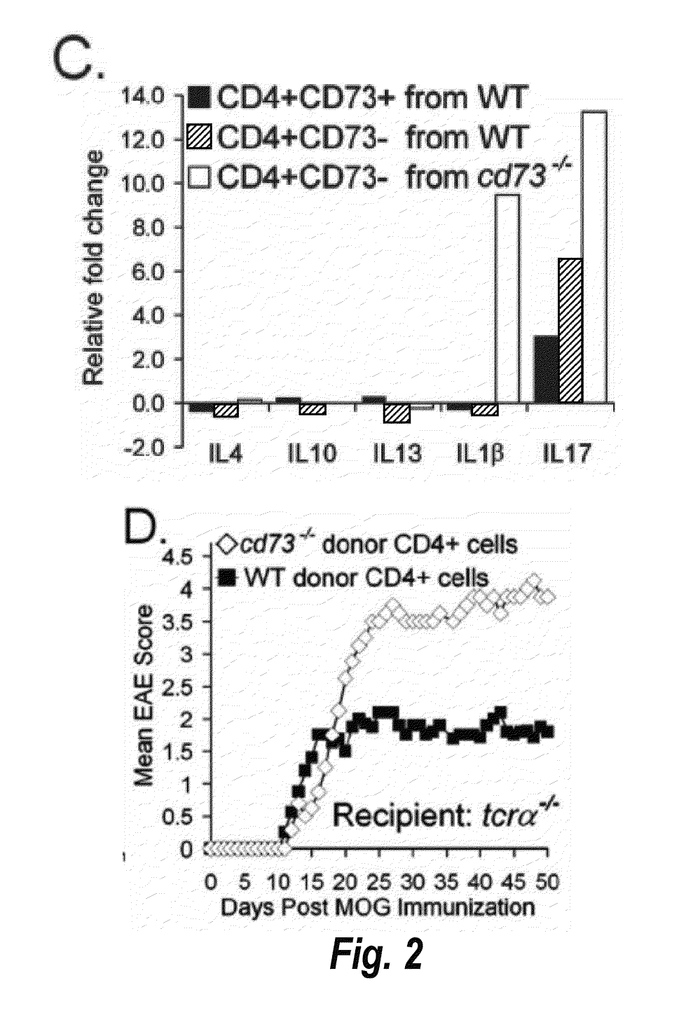

It was then asked whether the resistance of cd73^{-/-} mice to EAE induction could be explained by either an enhanced ability of cd73^{-/-} lymphocytes to suppress an immune response or an inability of these lymphocytes to respond to MOG stimulation. Naturally occurring CD4⁺CD25⁺FoxP3⁺ T cells, or Tregs, can regulate actively-induced EAE (Kohm et al., “Cutting Edge: CD4+CD25+ Regulatory T Cells Suppress Antigen-Specific Autoreactive Immune Responses and Central Nervous System Inflammation During Active Experimental Autoimmune Encephalomyelitis,” J. Immunol. 169:4712-4716 (2002), which is hereby incorporated by reference in its entirety). As Tregs have recently been shown to express CD73 and some reports suggest that the enzymatic activity of CD73 is needed for Treg function (Kobie et al., “T Regulatory and Primed Uncommitted CD4 T Cells Express CD73, Which Suppresses Effector CD4 T Cells by Converting 5′-Adenosine Monophosphate to Adenosine,” J. Immunol. 177:6780-6786); Deaglio et al., “Adenosine Generation Catalyzed by CD39 and CD73 Expressed on Regulatory T Cells Mediates Immune Suppression,” J. Exp. Med. 204:1257-1265 (2007), which are hereby incorporated by reference in their entirety), it was asked whether the number and suppressive activity of Tregs were normal in cd73^{-/-} mice. As shown in FIG. 2A, there were no significant differences in the frequencies of CD4⁺FoxP3⁺ Tregs in wild type and cd73 mice, either before or after EAE induction. Similarly, the percentage of CD4⁺ T cells that expressed CD73 changed only modestly after EAE induction in wild type mice (FIG. 2B). Additionally, no significant difference was observed in the suppressive capacity of wild type and cd73^{-/-} Tregs to inhibit MOG antigen-specific CD4⁺ effector T cell proliferation. To determine whether cd73^{-/-} T cells can respond when stimulated with MOG peptide, the capacity of these cells to proliferate and produce cytokines was assessed. CD4⁺ T cells from MOG-immunized cd73^{-/-} and wild type mice displayed similar degrees of in vitro proliferation in response to varying concentrations of MOG peptide. However, CD4⁺ T cells from MOG-immunized cd73 mice secreted higher levels of IL-17 and IL-1β following in vitro MOG stimulation, compared to those of wild type CD73CD4⁺ or CD73⁻CD4⁺ T cells (FIG. 2C). Elevated levels of IL-17 are associated with MS (Matusevicius et al., “Interleukin-17 mRNA Expression in Blood and CSF Mononuclear Cells is Augmented in Multiple Sclerosis,” Mult. Scler. 5:101-104 (1999), which is hereby incorporated by reference in its entirety) and EAE development (Komiyama et al., “IL-17 Plays an Important Role in the Development of Experimental Autoimmune Encephalomyelitis,” J. Immunol. 177:566-573 (2006), which is hereby incorporated by reference in its entirety), while high levels of the proinflammatory IL-1β cytokine are a risk factor for MS (de Jong et al., “Production of IL-1beta and IL-1Ra as Risk Factors for Susceptibility and Progression of Relapse-Onset Multiple Sclerosis,” J. Neuroimmunol. 126:172-179 (2002), which is hereby incorporated by reference in its entirety) and an enhancer of IL-17 production (Sutton et al., “A Crucial Role for Interleukin (IL)-1 in the Induction of IL-17-Producing T Cells That Mediate Autoimmune Encephalomyelitis,” J. Exp. Med. 203:1685-1691 (2006), which is hereby incorporated by reference in its entirety). No difference in IL-2, IL-4, IL-5, IL-10, IL-13, INF-γ and TNF-α secretion was observed between wild type and cd73^{-/-} T cells following MOG stimulation (FIG. 2C). Overall, the results from these assays suggest that cd73 T cells can respond well to MOG immunization.

It was then determined whether T cells from cd73^{-/-} mice possess the ability to cause EAE. To test this, CD4⁺ T cells from the spleen and lymph nodes of MOG immunized cd73^{-/-} mice were evaluated for their ability to induce EAE after transfer into tcrα^{-/-} (cd73^{+/+}) recipient mice. Tcrα^{-/-} a mice lack endogenous T cells and cannot develop EAE on their own (Elliott et al., “Mice Lacking Alpha Beta+T Cells are Resistant to the Induction of Experimental Autoimmune Encephalomyelitis,” J. Neuroimmunol. 70:139-144 (1996), which is hereby incorporated by reference in its entirety). Cd73^{+/+}tcrα^{-/-} recipient mice that received CD4⁺ T cells from cd73^{-/-} donors developed markedly more severe disease compared to those that received wild type CD4⁺ T cells (FIG. 2D). Wild type and cd73^{-/-} donor CD4⁺ T cells displayed equal degrees of expansion following transfer into cd73^{+/+} tcrα^{-/-} recipient mice. Thus, CD4⁺ T cells from cd73^{-/-} mice are not only capable of inducing EAE, but cause more severe EAE than those derived from wild type mice when transferred into cd73^{+/+} tcrα^{-/-} mice. These results are consistent with in vitro assays in which cd73^{-/-} CD4⁺ T cells secreted elevated levels of IL-17 and IL-1β (which are known to exacerbate EAE) in response to MOG stimulation (FIG. 2C) and suggest that cd73^{-/-} mice are resistant to MOG-induced EAE because of a lack of CD73^{-/-} expression in non-hematopoietic cells (most likely lack of expression in the CNS).

### Example 11

### Cd73^{-/-} Mice Exhibit Little/No Lymphocyte Infiltration into the CNS Following EAE Induction

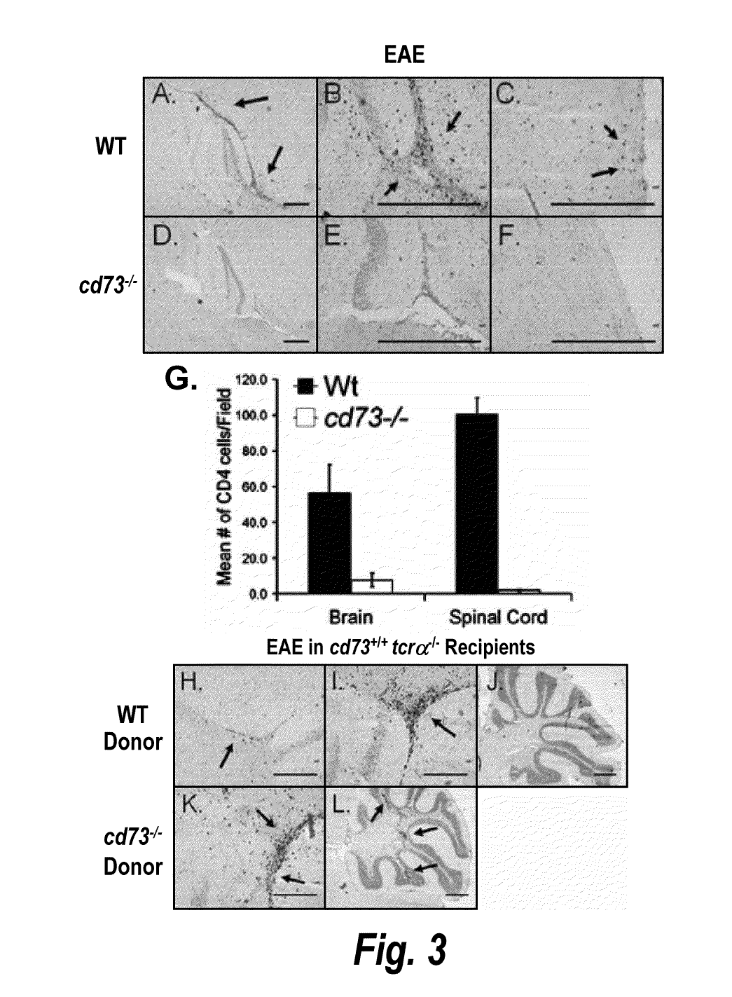

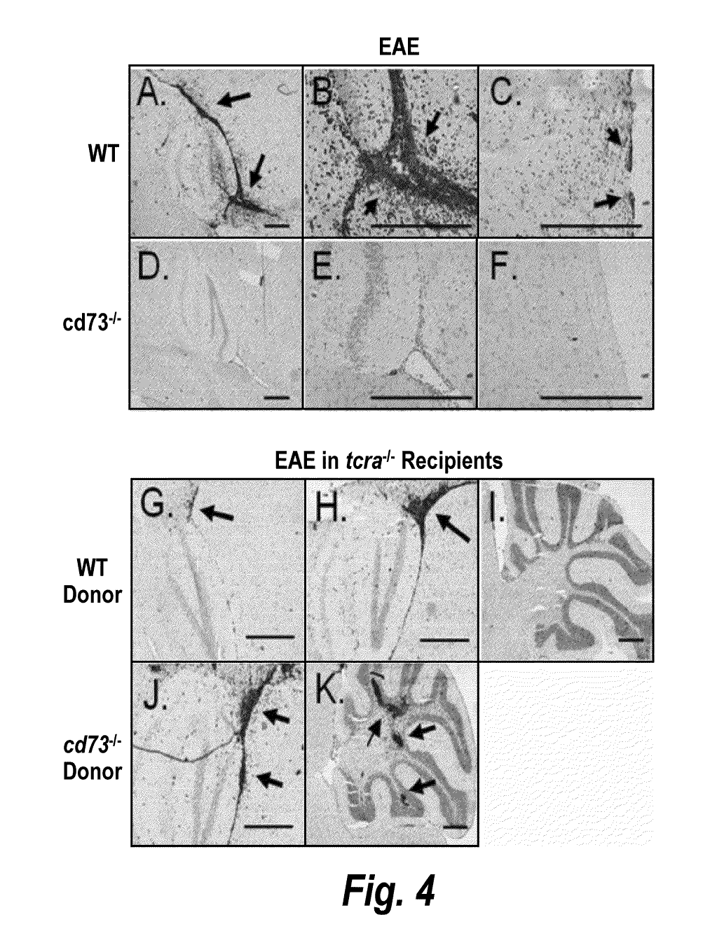

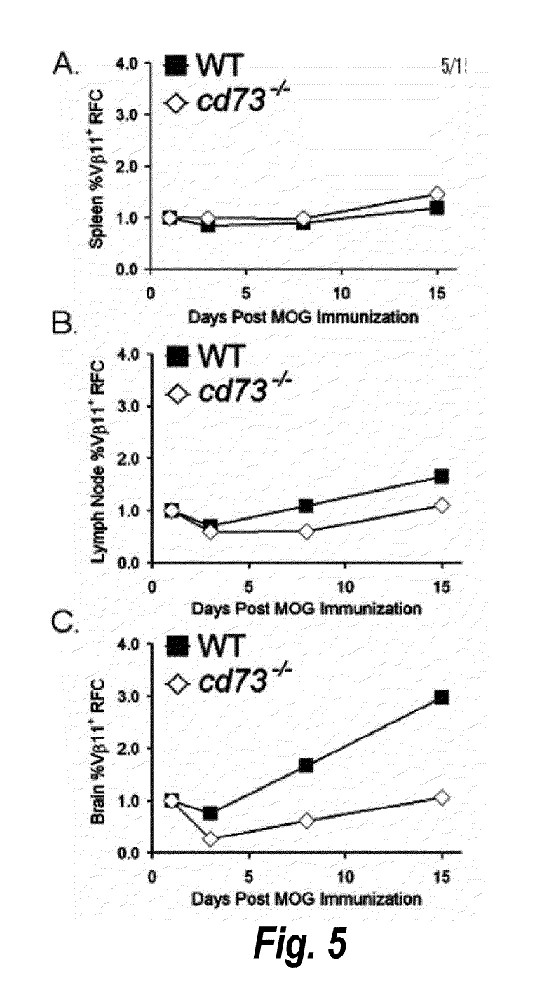

EAE is primarily a CD4⁺ T cell mediated disease (Montero et al., “Regulation of Experimental Autoimmune Encephalomyelitis by CD4+, CD25+ and CD8+ T Cells: Analysis Using Depleting Antibodies,” J. Autoimmun. 23:1-7 (2004), which is hereby incorporated by reference in its entirety) and during EAE progression, lymphocytes must first gain access into the CNS in order to mount their inflammatory response against CNS antigens, resulting in axonal demyelination and paralysis (Brown et al., “Time Course and Distribution of Inflammatory and Neurodegenerative Events Suggest Structural Bases for the Pathogenesis of Experimental Autoimmune Encephalomyelitis,” J. Comp. Neurol. 502:236-260 (2007), which is hereby incorporated by reference in its entirety). To determine if CNS lymphocyte infiltration is observed following EAE induction in cd73^{-/-} mice, brain and spinal cord sections were examined for the presence of CD4⁺ T cells and CD45⁺ cells by immunohistochemistry. Cd73^{-/-} mice displayed a dramatically lower frequency of CD4⁺ (FIGS. 3D-G) and CD45⁺ (FIG. 4 [Suppl. FIG. 1]) lymphocytes in the brain and spinal cord compared to wild type mice (FIGS. 3A-C, G) at day 13 post MOG immunization. Additionally, in lymphocyte tracking experiments where MOG-specific T cells from 2d2 TCR transgenic mice (Bettelli et al., “Myelin Oligodendrocyte Glycoprotein-Specific T Cell Receptor Transgenic Mice Develop Spontaneous Autoimmune Optic Neuritis,” J. Exp. Med. 197:1073-1081 (2003), which is hereby incorporated by reference in its entirety) were transferred into either wild type or cd73^{-/-} mice with concomitant EAE induction, the percentage of 2d2 cells in the CNS increased several fold with time in wild type recipient mice, but not at all in cd73^{-/-} recipients (FIG. 5). Overall, these results suggest that the observed protection against EAE induction in cd73^{-/-} mice is associated with considerably reduced CNS lymphocyte infiltration. Nevertheless, CD4⁺ T cells from MOG-immunized cd73^{-/-} mice possessed the ability to gain access to the CNS when transferred into cd73^{+/+}tcrα^{-/-} mice that were concomitantly induced to develop EAE (FIGS. 3K and 3L). In fact, earlier and more extensive CNS CD4⁺ lymphocyte infiltration was observed in cd73^{+/+}tcrα^{-/-} mice that received cd73^{-/-} CD4⁺ T cells (FIGS. 3K,L) than in those that received wild type CD4⁺ T cells (FIGS. 3H-J). Therefore, these results demonstrate that donor T cells from cd73^{-/-} mice have the ability to infiltrate the CNS of cd73^{+/+} recipient mice.

### Example 12

### CD73 Must be Expressed Either on Lymphocytes or in the CNS for Efficient EAE Development

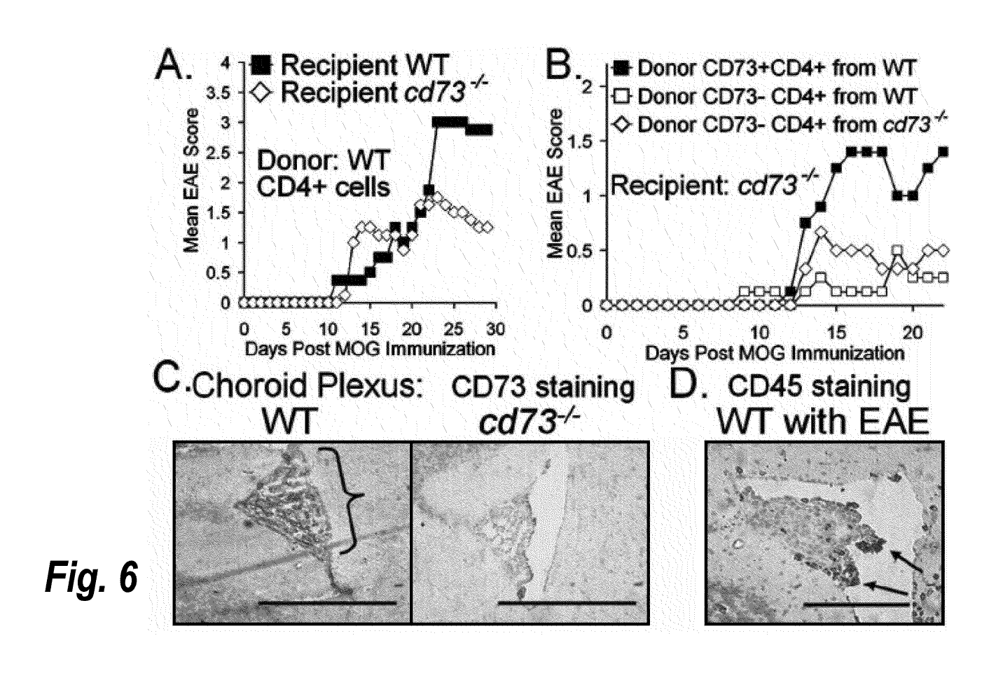

It was next asked whether CD73 expression on CD4⁺ T cells can compensate for a lack of CD73 expression in the CNS and allow the development of EAE. Therefore, CD4⁺ T cells were adoptively transferred from MOG-immunized wild type mice into cd73^{-/-} recipients, concomitantly induced EAE, and compared disease activity with that of similarly treated wild type recipients (FIG. 6A). While wild type recipients developed disease following EAE induction as expected, cd73^{-/-} recipients also developed prominent EAE with an average disease score of 1.5 by three weeks after disease induction. This was much higher than the 0.5 average score that cd73^{-/-} mice normally showed at this same time point (FIG. 1). To further define the association of CD4⁺ T cell CD73 expression with EAE susceptibility, sorted CD73⁺CD4⁺ and CD73CD4⁺ T cells from immunized wild type mice, or total CD4⁺ (CD73⁻) T cells from immunized cd73 mice, were transferred into cd73 recipients with concomitant EAE induction (FIG. 6B). Cd73^{-/-} mice that received CD73⁺CD4⁺ T cells from wild type mice developed EAE with an average score of approximately 1.5 at three weeks post induction. Conversely, cd73 mice that received wild type derived CD73⁻CD4⁺ T cells did not develop significant EAE. Additionally, CD4⁺ cells from cd73^{-/-} mice, which have the ability to cause severe EAE in CD73-expressing tcrα^{-/-} mice (FIG. 2D), were also incapable of potentiating EAE in recipient cd73^{-/-} mice (FIG. 6B). Therefore, although CD73 expression on T cells can partially compensate for a lack of CD73 expression in non-hematopoietic cells, EAE is most efficiently induced when CD73 is expressed in both compartments.

The identity of the CD73-expressing non-hematopoietic cells that promote the development of EAE is not known. Vascular endothelial cells at the BBB were considered as likely candidates, as many types of endothelial cells express CD73 (Yamashita et al., “CD73 Expression and Fyn-Dependent Signaling on Murine Lymphocytes,” Eur. J. Immunol. 28:2981-2990 (1998), which is hereby incorporated by reference in its entirety). However, immunohistochemistry revealed that mouse brain endothelial cells are CD73⁻. During these experiments, it was observed that CD73 is, however, highly expressed in the brain on the choroid plexus (FIG. 6C), which is an entry point into the CNS for lymphocytes during EAE progression (Brown et al., “Time Course and Distribution of Inflammatory and Neurodegenerative Events Suggest Structural Bases for the Pathogenesis of Experimental Autoimmune Encephalomyelitis,” J. Comp. Neurol. 502:236-260 (2007), which is hereby incorporated by reference in its entirety). FIG. 4D shows infiltrating lymphocytes in association with the choroid plexus of wild type mice 12 days post-EAE induction. Minimal CD73 staining was also observed on submeningeal regions of the spinal cord. Taken together, these results suggest that CD73 expression, whether on T cells or in the CNS (perhaps on the choroid plexus), is necessary for efficient EAE development.

### Example 13

### Adenosine Receptor Antagonists Protect Mice Against EAE Induction

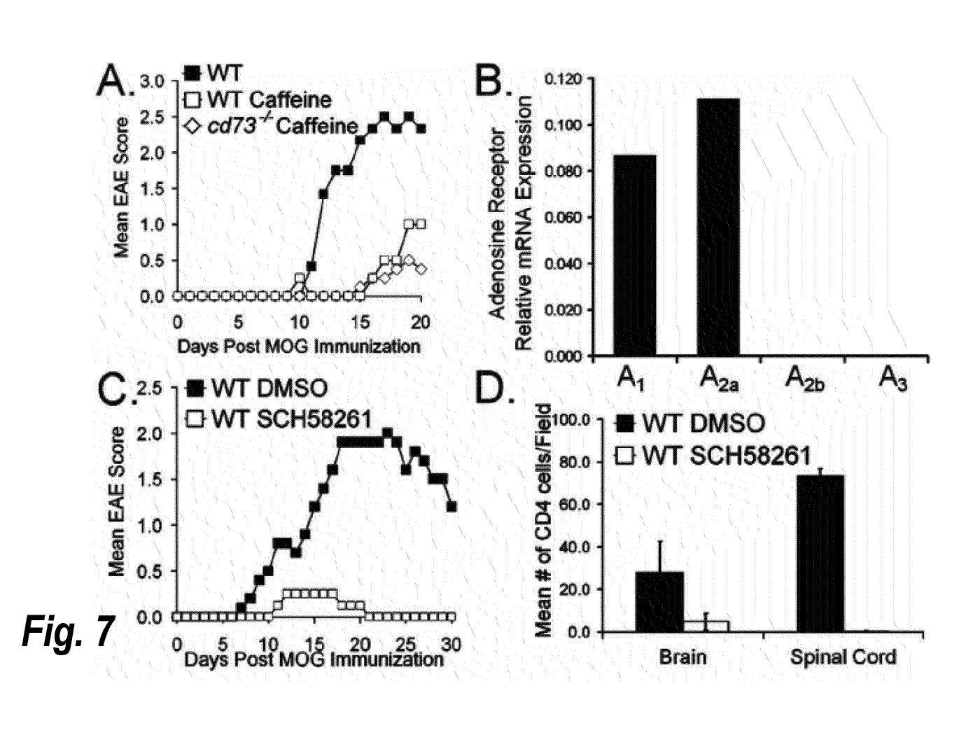

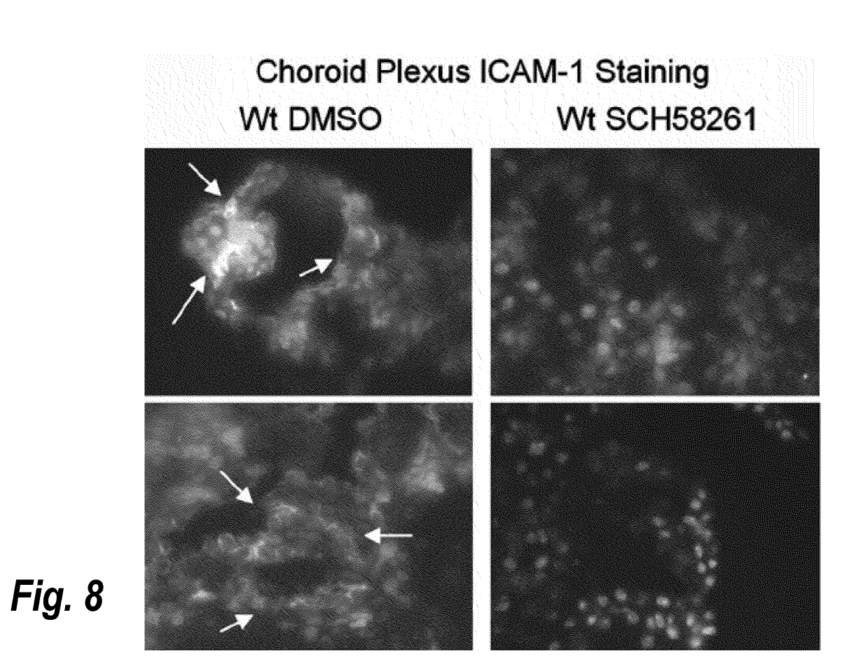

As CD73 catalyzes the breakdown of AMP to adenosine and ARs are expressed in the CNS (Tsutsui et al., “A1 Adenosine Receptor Upregulation and Activation Attenuates Neuroinflammation and Demyelination in a Model of Multiple Sclerosis,” J. Neurosci. 24:1521-1529 (2004)); Rosi et al., The Influence of Brain Inflammation Upon Neuronal Adenosine A2B Receptors,” J. Neurochem. 86:220-227 (2003), which are hereby incorporated by reference in their entirety), it was next determined if AR signaling is important during EAE progression. Wild type and cd73^{-/-} mice were treated with the broad spectrum AR antagonist caffeine (Dall'Igna et al., “Caffeine as a Neuroprotective Adenosine Receptor Antagonist,” Ann. Pharmacother. 38:717-718 (2004), which is hereby incorporated by reference in its entirety) at 0.6 g/L in their drinking water, which corresponds to an approximate dose of 4.0 mg/mouse of caffeine per day (Johansson et al., “A1 and A2A Adenosine Receptors and A1 mRNA in Mouse Brain: Effect of Long-Term Caffeine Treatment,” Brain Res. 762:153-164 (1997), which is hereby incorporated by reference in its entirety), 1 day prior to and throughout the duration of the EAE experiment (FIG. 7A). Wild type mice that received caffeine were dramatically protected against EAE development (FIG. 7A), comparable to previously published results (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety). As expected, cd73^{-/-} mice that received caffeine did not develop EAE (FIG. 7A). Since CD73 is highly expressed on the choroid plexus (FIG. 6C), it was next determined if ARs are also expressed on the choroid plexus. Utilizing the Z310 murine choroid plexus cell line (Zheng et al., “Establishment and Characterization of an Immortalized Z310 Choroidal Epithelial Cell Line from Murine Choroid Plexus,” Brain Res. 958:371-380 (2002), which is hereby incorporated by reference in its entirety), only mRNA for the A1 and A2A adenosine receptor subtypes were detected by qPCR (FIG. 7B). As A1AR^{-/-} mice have been previously shown to develop severe EAE following disease induction (Tsutsui et al., “A₁ Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety), it was asked if treatment of wild type mice with SCH58261 (Melani et al., “The Selective A_{2A} Receptor Antagonist SCH 58261 Protects From Neurological Deficit, Brain Damage and Activation of p38 MAPK in Rat Focal Cerebral Ischemia,” Brain Res. 1073-1074:470-480 (2006), which is hereby incorporated by reference in its entirety), an AR antagonist specific for the A_{2A} subtype, could protect against EAE development. Wild type mice were given 1 mg/kg of SCH58261 in DMSO or DMSO alone both i.p. and s.c. (for a total of 2 mg/kg) 1 day prior to EAE induction and daily for 30 days throughout the course of the experiment (FIG. 7C). Wild type mice that received SCH58261 were dramatically protected against EAE development compared to wild type mice that received DMSO alone (FIG. 7C). Additionally, wild type mice given SCH58261 displayed a significantly lower frequency of CD4⁺ lymphocytes in the brain and spinal cord compared to DMSO treated wild type mice at day 15 post-EAE induction (FIG. 7D). As studies have shown that adhesion molecules (such as ICAM-1, VCAM-1, and MadCAM-1) on the choroid plexus play a role in the pathogenesis of EAE (Engelhardt et al., “Involvement of the Choroid Plexus in Central Nervous System Inflammation,” Microsc. Res. Tech. 52:112-129 (2001), which is hereby incorporated by reference in its entirety), it was determined if SCH58261 treatment affected adhesion molecule expression on the choroid plexus following EAE induction. Comparison of the choroid plexus from DMSO and SCH58261 treated wild type mice shows that A2A AR blockade prevented the up regulation of ICAM-1 that normally occurs during EAE progression (FIG. 8).

Based on these results, it was concluded that the inability of cd73^{-/-} mice to catalyze the generation of extracellular adenosine explains their failure to efficiently develop EAE following MOG immunization and that CD73 expression and A2A AR signaling at the choroid plexus are requirements for EAE progression.

The goal of this study was to evaluate the role of CD73 in EAE, an animal model for MS. As CD73 catalyzes the formation of extracellular adenosine which is usually immunosuppressive (Boors et al., “Adenosine 5′-Triphosphate and Adenosine as Endogenous Signaling Molecules in Immunity and Inflammation,” Pharmacol. Ther. 112:358-404 (2006), which is hereby incorporated by reference in its entirety) and A1AR^{-/-} mice exhibit severe EAE (Tsutsui et al., “A₁ Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety), applicants predicted that cd73^{-/-} mice would also develop severe EAE. However, cd73^{-/-} mice were highly resistant to EAE induction, a surprising finding considering the plethora of studies demonstrating that cd73^{-/-} mice are more prone to inflammation. For example, cd73^{-/-} mice are more susceptible to bleomycin-induced lung injury (Volmer et al., “Ecto-5′-Nucleotidase (CD73)-Mediated Adenosine Production is Tissue Protective in a Model of Bleomycin-Induced Lung Injury,” J. Immunol. 176:4449-4458 (2006), which is hereby incorporated by reference in its entirety) and are more prone to vascular inflammation and neointima formation (Zernecke et al., “CD73/ecto-5′-Nucleotidase Protects Against Vascular Inflammation and Neointima Formation,” Circulation 113:2120-2127 (2006), which is hereby incorporated by reference in its entirety). Consistent with these reports, applicants showed that cd73^{-/-} T cells produced higher levels of the EAE-associated proinflammatory cytokines IL-1β and IL-17 following MOG stimulation. Furthermore, the adoptive transfer of cd73^{-/-} T cells to tcrα^{-/-} mice, which lack T cells but express CD73 throughout their periphery, resulted in severe CNS inflammation following MOG immunization, consistent with a role for adenosine as an anti-inflammatory mediator. It is interesting to note that IFN-β treatment, one of the most effective therapies for MS, has been shown to up regulate CD73 expression on endothelial cells both in vitro and in vivo (Airas et al., “Mechanism of Action of IFN-Beta in the Treatment of Multiple Sclerosis: A Special Reference to CD73 and Adenosine,” Ann. N.Y. Acad. Sci. 1110:641-648 (2007), which is hereby incorporated by reference in its entirety). Thus, although the mechanism by which IFN-β benefits MS patients is incompletely understood, increased production of adenosine accompanied by its known anti-inflammatory and neuroprotective effects could be a factor.

Consistent with their resistance to EAE induction, cd73^{-/-} mice had a lower frequency of cells infiltrating the CNS during EAE compared to wild type mice. This was also an unexpected finding, as CD73-generated adenosine has previously been shown to restrict the migration of neutrophils across vascular endothelium during hypoxia and of lymphocytes across high endothelial venules of draining lymph nodes (Thompson et al., “Crucial Role for Ecto-5′-Nucleotidase (CD73) in Vascular Leakage During Hypoxia,” J. Exp. Med. 200:1395-1405 (2004), which is hereby incorporated by reference in its entirety). Applicants' data, in contrast, suggest that CD73, and the extracellular adenosine generated by CD73, are needed for the efficient passage of pathogenic T cells into the CNS. Therefore, the role that CD73 and adenosine play in CNS lymphocyte infiltration during EAE is more profound than their role in modulation of neuroinflammation.

It is important to know where CD73 must be expressed for T cell migration into the CNS. CD73 is found on subsets of T cells (Yamashita et al., “CD73 Expression and Fyn-Dependent Signaling on Murine Lymphocytes,” Eur. J. Immunol. 28:2981-2990 (1998), which is hereby incorporated by reference in its entirety) as well as on some epithelial (Strohmeier et al., “Surface Expression, Polarization, and Functional Significance of CD73 in Human Intestinal Epithelia,” J. Clin. Invest. 99:2588-2601 (1997), which is hereby incorporated by reference in its entirety) and endothelial cells (Yamashita et al., “CD73 Expression and Fyn-Dependent Signaling on Murine Lymphocytes,” Eur. J. Immunol. 28:2981-2990 (1998), which is hereby incorporated by reference in its entirety). The data presented here clearly demonstrates that although cd73^{-/-} T cells respond well to MOG immunization, they cannot enter the CNS unless CD73 is expressed in non-hematopoietic tissues (i.e. cd73^{+/+}tcrα^{-/-} mice which develop EAE after adoptive transfer of CD4⁺ T cells from cd73^{-/-} mice). A lack of CD73 on non-hematopoietic cells can also be compensated for, in part, by CD73 expression on T cells (i.e., cd73^{-/-} mice become susceptible to EAE when CD73⁺, but not CD73⁻, CD4⁺ T cells are adoptively transferred). While BBB endothelial cells as a relevant source of CD73 in the CNS were considered, because CD73 is expressed on human brain endothelial cells (Airas et al., “Mechanism of Action of IFN-Beta in the Treatment of Multiple Sclerosis: A Special Reference to CD73 and Adenosine,” Ann. N.Y. Acad. Sci. 1110:641-648 (2007), which is hereby incorporated by reference in its entirety), immunohistochemistry revealed that mouse brain endothelial cells are CD73⁻. However, CD73 was found to be highly expressed on choroid plexus epithelial cells, which form the barrier between the blood and the cerebrospinal fluid (CSF) and have a role in regulating lymphocyte immunosurveillance in the CNS (Steffen et al., “CAM-1, VCAM-1, and MAdCAM-1 are Expressed on Choroid Plexus Epithelium but Not Endothelium and Mediate Binding of Lymphocytes In Vitro,” Am. J. Pathol. 148:1819-1838 (1996), which is hereby incorporated by reference in its entirety). The choroid plexus has also been suggested to be the entry point for T cells during the initiation of EAE progression (Brown et al., “Time Course and Distribution of Inflammatory and Neurodegenerative Events Suggest Structural Bases for the Pathogenesis of Experimental Autoimmune Encephalomyelitis,” J. Comp. Neurol. 502:236-260 (2007), which is hereby incorporated by reference in its entirety). While the role of lymphocyte-brain endothelial cell interactions via VLA-4NCAM-1 binding in both EAE and MS is well-documented (Rice et al., “Anti-Alpha4 Integrin Therapy for Multiple Sclerosis Mechanisms and Rationale,” Neurology 64:1336-1342 (2005), which is hereby incorporated by reference in its entirety), perhaps lymphocyte trafficking across the endothelial BBB is more important for disease maintenance and progression than for disease initiation, at least in EAE.

The next issue is how CD73 facilitates the migration of T cells into the CNS. Earlier work showed that lymphocyte CD73 can promote the binding of human lymphocytes to endothelial cells in an LFA-1-dependent fashion (Airas et al., “CD73 Engagement Promotes Lymphocyte Binding to Endothelial Cells Via a Lymphocyte Function-Associated Antigen-1-dependent Mechanism,” J. Immunol. 165:5411-5417 (2000), which is hereby incorporated by reference in its entirety). This does not appear to be the function of CD73 in EAE, however, becuase CD73-deficient T cells can enter the CNS and cause severe disease in cd73^{+/+}tcrα^{-/-} mice (FIG. 2D). Alternatively, CD73 can function as an enzyme to produce extracellular adenosine, a ligand for cell surface ARs. It is this latter function that is relevant for the current work given that AR blockade with caffeine or SCH58261 can protect mice from EAE. While the broad spectrum AR antagonist caffeine also inhibits certain phosphodiesterases (Choi et al., “Caffeine and Theophylline Analogues: Correlation of Behavioral Effects With Activity as Adenosine Receptor Antagonists and as Phosphodiesterase Inhibitors,” Life Sci. 43:387-398 (1988), which is hereby incorporated by reference in its entirety), its modulation of EAE progression is most likely through its effect on AR signaling (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety). This notion is supported by the fact that SCH58261 also prevents EAE progression by specifically inhibiting A2A AR signaling. As CD73 and the A1 and A2A AR subtypes are expressed on the choroid plexus, extracellular adenosine produced by CD73 at the choroid plexus can signal in an autocrine fashion.

Adenosine signaling most likely regulates the expression of adhesion molecules at the choroid plexus. Studies have shown that the up regulation of the adhesion molecules ICAM-1, VCAM-1, and MadCAM-1 at the choroid plexus are associated with EAE progression (Engelhardt et al., Involvement of the Choroid Plexus in Central Nervous System Inflammation,” Microsc. Res. Tech. 52:112-129 (2001), which is hereby incorporated by reference in its entirety). As mice treated with the A2A AR antagonist SCH58261 do not experience increased choroid plexus ICAM-1 expression (FIG. 8), as normally occurs following EAE induction (Engelhardt et al., “Involvement of the Choroid Plexus in Central Nervous System Inflammation,” Microsc. Res. Tech. 52:112-129 (2001), which is hereby incorporated by reference in its entirety), the present results suggest that A2A AR signaling increases ICAM-1 during EAE progression.

In summary, this data shows that CD73 plays a critical role in the progression of EAE. Mice that lack CD73 are protected from the degenerative symptoms and CNS inflammation that are associated with EAE induction. This is the first study to demonstrate a requirement for CD73 expression and AR signaling for the efficient entry of lymphocytes into the CNS during EAE. The data presented here may mark the first steps of a journey that will lead to new therapies for MS and other neuroinflammatory diseases.

### Example 14

### The BBB Can be Modulated Through Activation of the Adenosine Receptors

The objective of this experiment was to determine if the blood brain barrier could be modulated by activation of adenosine receptors. NECA is a non-selective adenosine receptor agonist, with similar affinities for A₁, A_{2A} and A₃ adenosine receptors and a low affinity for the A_{2b} adenosine receptor. In order to determine if activation of adenosine receptors would induce extravasation of Evans Blue dye across the blood brain barrier (BBB), mice were treated with: NECA, a non-selective adenosine receptor agonist (n=5, 100 μl 0.01 nM); SCH58261, an A_{2A} adenosine receptor specific antagonist (n=5, 1 mg/kg); pertussis toxin, an agent known to induce BBB leakiness and as such used as a positive control (n=7, 200 μl); and, PBS as a vehicle control (n=5, 100 μl). CD73^{-/-} mice, which lack the ability to produce extracellular adenosine, were also treated with NECA (n=4, 100 μl 0.01 nM). Treatments were administered as a single i.v. injection one hour prior to i.v. injection of 200 μl 1% Evans Blue dye (2 μg total dye injected). Four hours after administration of Evans Blue, mice were anesthetized with a ketamine/xylazine mix and perfused via the left ventricle with ice cold PBS. Brains were harvested and homogenized in n,n-dimethylformamide (DMF) at 5 μl/mg (v:w). Tissue was incubated for 72 hours at room temperature in DMF to extract the dye. Following extraction, the tissue/solvent mixture was centrifuged at 500×g for 30 minutes and 100 μl of supernatant was read on a BioTex spectrophotometer at 620 nm. Data is expressed as μg Evans Blue/ml DMF.

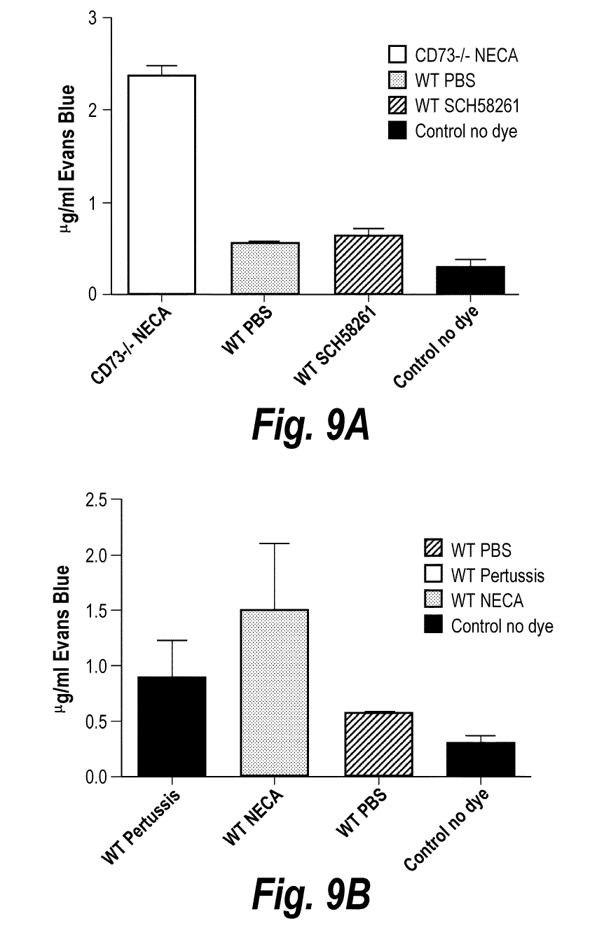

Treating mice with the general adenosine receptor agonist NECA can induce migration of dye across the blood brain barrier. This suggests that this barrier can be modulated through activation of the adenosine receptors. In FIG. 9A, CD73^{-/-} mice, which lack extracellular adenosine and thus cannot adequately signal through adenosine receptors, were treated with NECA, resulting in an almost five fold increase in dye migration vs. the PBS control. SCH58261 was used as a negative control since applicants have shown that blocking of the A_{2A} adenosine receptor using this antagonist can prevent lymphocyte entry into the brain (Mills et al., “CD73 is Required for Efficient Entry of Lymphocytes into the Central Nervous System During Experimental Autoimmune Encephalomyelitis,” Proc. Natl. Acad. Sci. 105(27):9325-9330 (2008), which is hereby incorporated by reference in its entirety). In FIG. 9B, WT mice treated with NECA also show an increase over control mice. Pertussis is used as a positive control, as it is known to induce blood brain barrier leakiness in the mouse EAE model.

### Example 15

### The A_{2A} and A_{2b} Adenosine Receptors are Expressed on the Human Endothelial Cell Line hCMEC/D3

In order to establish an in vitro blood brain barrier (BBB), the human brain endothelial cell line hCMEC/D3 (Weksler et al., “Blood-brain Barrier-specific Properties of a Human Adult Brain Endothelial Cell Line,” J. Neurochem. 19(13):1872-4 (2005); Poller et al., “The Human Brain Endothelial Cell Line hCMEC/D3 as a Human Blood-brain Barrier Model for Drug Transport Studies,” J. Neurochem. 107(5):1358-1368 (2008), which are hereby incorporated by reference in their entirety) was obtained, which has been previously described as having BBB properties. Here, expression pattern of adenosine receptors on these cells was established.

hCMEC/D3 cells were grown to confluence, harvested and RNA was extracted using TRIzol reagent (Invitrogen, Carlsbad, Calif.) according to the manufacturer's instructions. cDNA was synthesized using a Verso cDNA kit (Thermo Scientific, Waltham, Mass.), and Real Time PCR was performed using Power SYBR Green (Applied Biosystems, Foster City, Calif.).

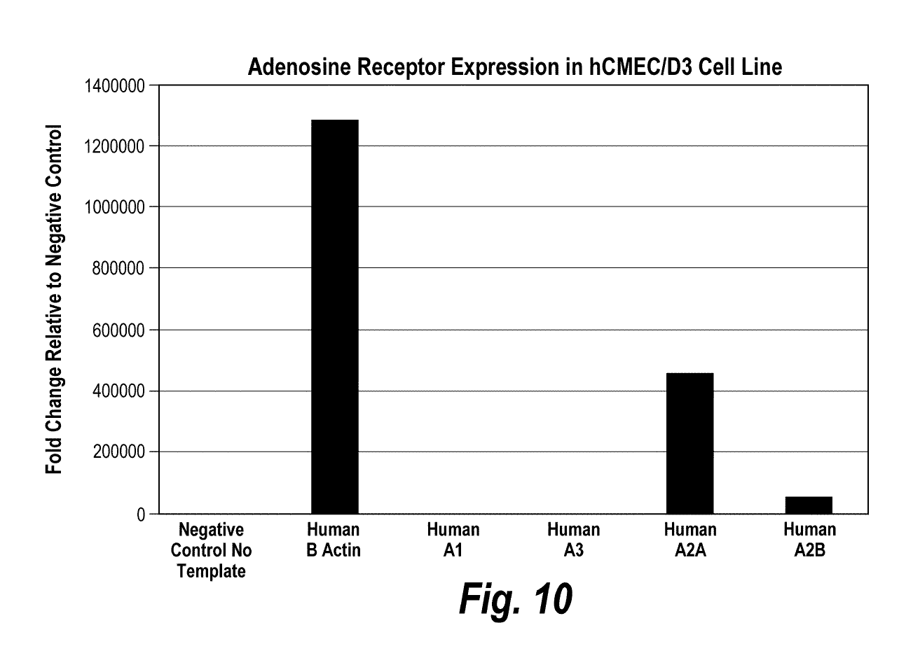

As shown in FIG. 10, the A_{2A} and A_{2b} adenosine receptors were found to be expressed on the human endothelial cell line hCMEC/D3.

### Example 16

### Adenosine Receptor Stimulation of Brain Endothelial Cells Promotes Lymphocyte Migration Through the BBB

The blood brain barrier (“BBB”) is comprised of endothelial cells. During late stages of EAE, lymphocytes are known to cross the BBB. In order to determine if adenosine receptor stimulation of brain endothelial cells could promote lymphocyte migration through the BBB, an in vitro BBB was established. The human brain endothelial cell line hCMEC/D3 (Weksler et al., “Blood-brain Barrier-specific Properties of a Human Adult Brain Endothelial Cell Line,” J. Neurochem. 19(13):1872-4 (2005); Poller et al., “The Human Brain Endothelial Cell Line hCMEC/D3 as a Human Blood-brain Barrier Model for Drug Transport Studies,” J. Neurochem. 107(5):1358-1368 (2008), which are hereby incorporated by reference in their entirety) was obtained, which has been previously described as having BBB properties.

hCMEC/D3 cells were seeded onto Transwell and allowed to grow to confluencey. 2×10⁶ Jurkat cells were added to the upper chamber with or without NECA (general adenosine receptor [AR] agonist), CCPA (A₁ AR agonist), CGS 21860 (A_{2A} AR agonist), or DMSO vehicle. After 24 hours, migrated cells in the lower chamber were counted. Values are relative to the number of cells that migrate through non-HCMECD3 seeded transwells.

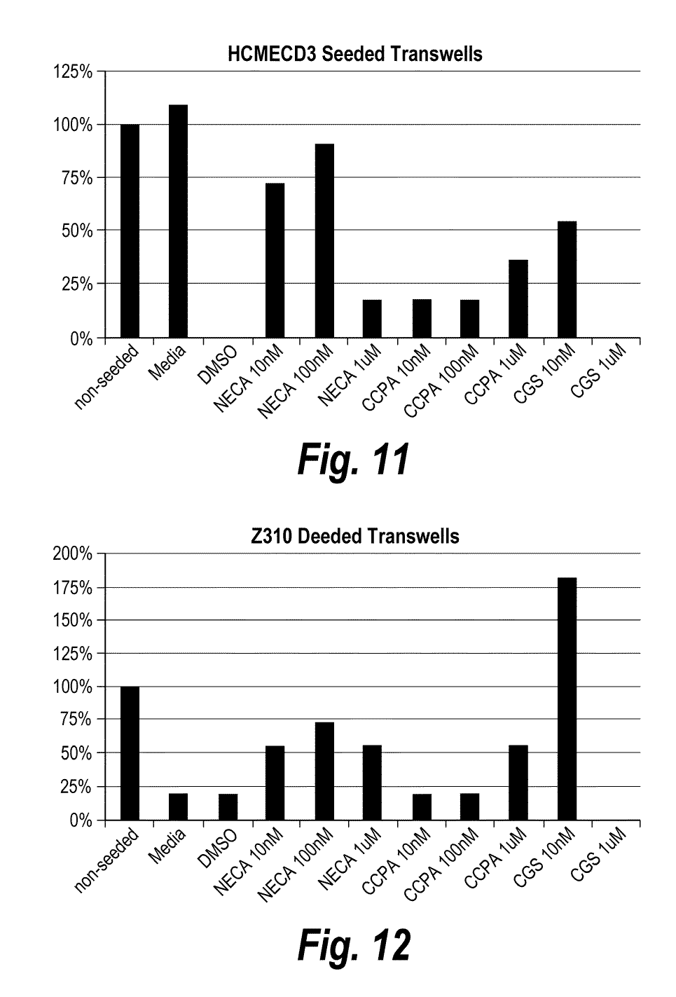

As shown in FIG. 11, NECA, a broad spectrum adenosine receptor agonist, induced some migration. CGS, the A_{2A} adenosine receptor agonist, promoted lymphocyte migration across the in vitro BBB when used at a lower concentration. CCPA, the A₁ agonist, induced lymphocyte migration at high levels possibly due to activation of the A_{2A} adenosine receptor, which has a lower affinity for CCPA and thus is only activated at higher levels of CCPA.

### Example 17

### A_{2A} Adenosine Receptor Activation Promotes Lymphocyte Migration Across the CP

The choroid plexus (“CP”) controls lymphocyte migration into the CNS. The CP expresses the A₁ and A_{2A} adenosine receptors. EAE is prevented in mice when A2A adenosine receptor activity is blocked. EAE is enhanced when the A₁ adenosine receptor is missing. It was hypothesized that A2A adenosine receptor activation promotes lymphocyte migration across the CP. Z310 cells are a murine choroid plexus cell line.

To test the hypothesis, Transwell membranes were seeded with Z310 cells and allowed to grow to confluencey. 2×10⁶ Jurkat cells were added to the upper chamber with or with out NECA (n=1, general AR agonist), CCPA (n=1, AAR agonist), CGS 21860 (n=1, A_{2A}AR agonist), or DMSO vehicle (n=1). After 24 hours, migrated cells in the lower chamber were counted. Values are relative to the number of cells that migrate through non-Z310 seeded transwells and the results are shown in FIG. 12.

As shown in FIG. 12, NECA, a broad spectrum adenosine receptor agonist, induced migration. CGS, the A_{2A} adenosine receptor agonist, promoted lymphocyte migration across the CP. CCPA, the A₁ agonist, induced lymphocyte migration at high levels possibly due to activation of the A_{2A} adenosine receptor, which has a lower affinity for CCPA and as such is only activated at high levels of CCPA.

### Example 18

### Human Brain Endothelial Cells are Sensitive to Adenosine Receptor Induced cAMP Regulation

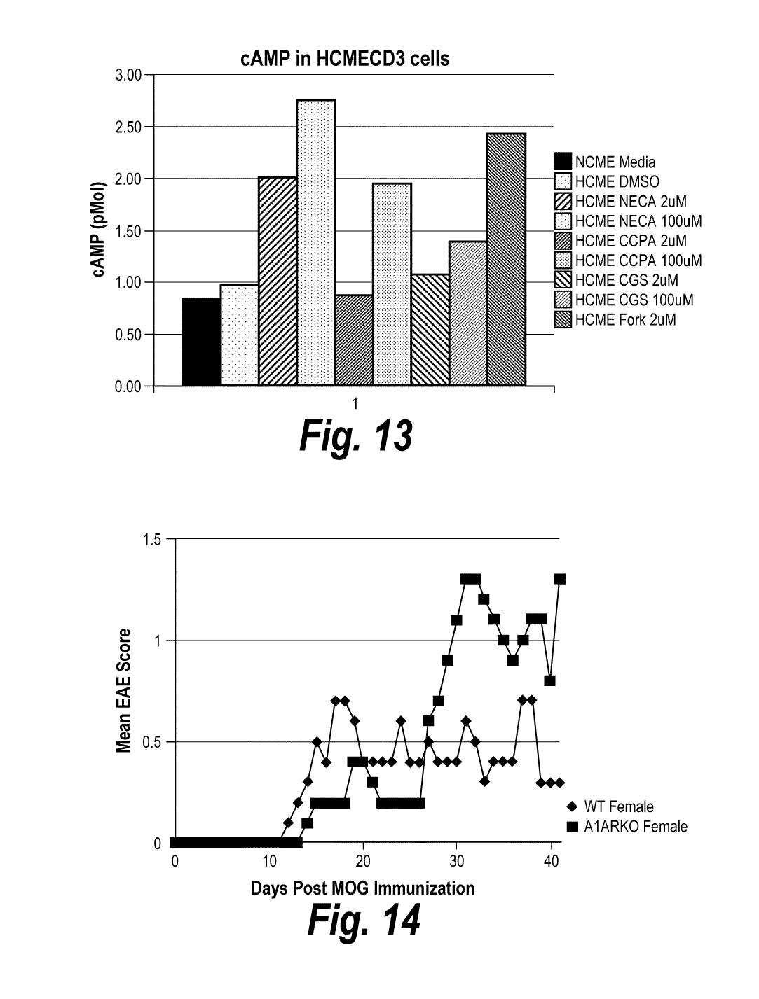

Adenosine receptor activation regulates cAMP levels in cells. In order to determine the sensitivity of human brain endothelial cells to adenosine receptor induced cAMP regulation, human brain endothelial cells were cultured with adenosine receptor agonists at various concentrations, followed by cAMP level analysis, as shown in FIG. 13.

HCMECD3 cells were grown to confluencey on 24 well plates. As adenosine receptor (“AR”) stimulation is known to influence cAMP levels, cells were treated with or without various concentrations of NECA (general AR agonist), CCPA (A₁ AR agonist), CGS 21860 (A_{2A} AR agonist), DMSO vehicle, or Forksolin (induces cAMP). After 15 minutes, lysis buffer was added and the cells were frozen at −80 C to stop the reaction. Duplicate samples were used for each condition. cAMP levels were assayed using a cAMP Screen kit (Applied Biosystems, Foster City, Calif.).

As shown in FIG. 13, the broad spectrum adenosine receptor agonist NECA increased cAMP levels, verifying that these cells can respond to adenosine receptor signaling. High levels of CCPA, the A₁ adenosine receptor agonist, increased cAMP levels, again perhaps due to activation of the A_{2A} adenosine receptor, which has a lower affinity for CCPA and as such is only activated at high levels of CCPA. CGS, the A_{2A} adenosine receptor agonist slightly increased cAMP levels in the human brain endothelial cell line.

### Example 19

### Female A1 Adenosine Receptor Knockout Mice Develop More Severe EAE Than Wild Type

A₁ and A_{2A} adenosine receptors are expressed on the choroid plexus. A_{2A} adenosine receptor antagonists protect mice from EAE. Are mice that lack the A₁ adenosine receptor prone to development of more severe EAE than wild type controls? To answer this question, disease profiles of wild type and A₁ adenosine receptor null mice were compared.

Female A₁ adenosine receptor knockout (A1ARKO, n=5) and wild type (WT, n=5) mice were immunized with CFA/MOG₃₅₋₅₅+PTX on Dec. 2, 2008 and scored daily for 41 days. As the results in FIG. 14 illustrate, A₁ARKO mice develop more severe EAE than WT, and also develop disease at a faster rate than WT.

### Example 20

### Brains From Wild Type Mice Fed an Adenosine Receptor Antagonist Have Higher Levels of FITC-Dextran Than Brains from CD73^{-/-} Mice Fed an Adenosine Receptor Antagonist

In order to examine the effects of caffeine, a general adenosine receptor antagonist, on blood brain barrier permeability, mice were fed caffeine for several days and then injected with FITC Dextran, commonly used to assess endothelial permeability.

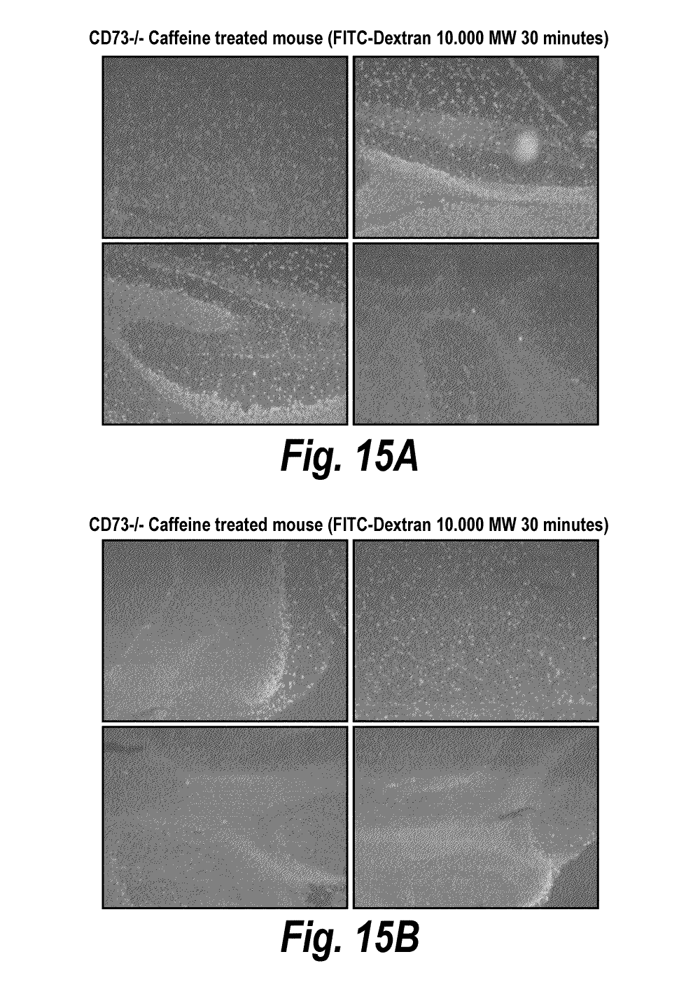

More particularly, mice were fed 0.6 g/l caffeine (Sigma, St. Louis, Mo.) in water or regular water ad lib for five days. Mice were injected IP with FITC Dextran (10,000 MW, Molecular Probes, Eugene, Oreg.) and after 30 minutes mice were perfused with ice cold PBS via the left ventricle. Brains were removed and snap frozen in OCT (Tissue Tek, Torrance, Calif.) and stored at −80° C. until sectioning. Tissue sections (5 μm) were stained with hematoxylin for light microscopy and with DAPI for a fluorescent counterstain. The results are shown in FIG. 15.

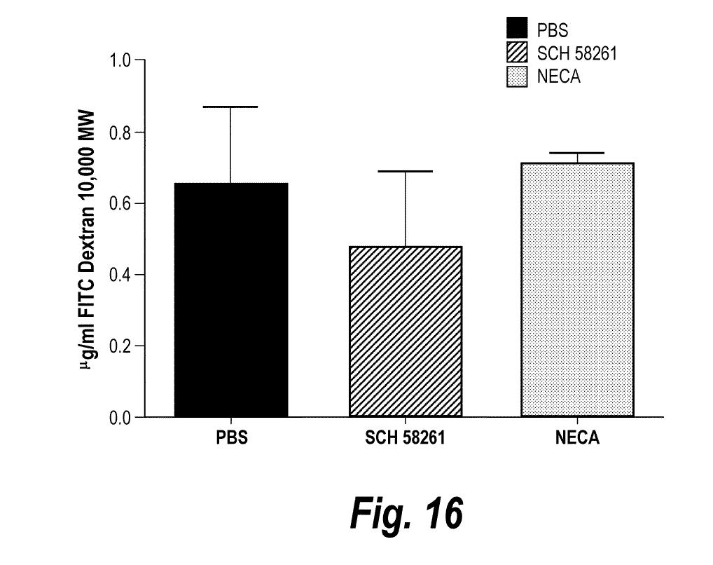

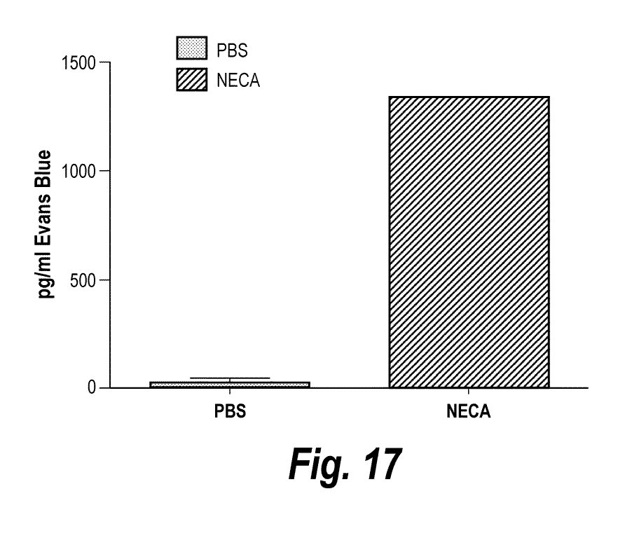

As shown in FIG. 15A, visualization of brain sections from CD73^{-/-} mice fed caffeine displayed a much less intense green color than wild type mice, indicating less FITC-Dextran extravasation across the blood brain barrier. Brain sections from wild type mice displayed an intensely green background (FIG. 15B) that is indicative of more FITC-dextran extravasation across the blood brain barrier. FIG. 16 shows the results for wild-type mice in graphical form.

### Example 21

### Adenosine Receptor Agonist NECA Increases Evans Blue Dye Extravasation Across the Blood Brain Barrier

The objective of this experiment was to determine if the blood brain barrier could be modulated by activation of adenosine receptors. NECA is a non-selective adenosine receptor agonist, with similar affinities for A₁, A_{2A} and A₃ adenosine receptors and a low affinity for the A_{2B} adenosine receptor.

In order to determine if activation of adenosine receptors would induce extravazation of Evans Blue dye across the blood brain barrier (BBB), mice were first treated on day one with NECA, a non-selective adenosine receptor agonist (n=2, 100 μl 0.01 nM); and, PBS as a vehicle control (n=2, 100 μl). On day 2 mice were then immunized with CFA-MOG₃₅₋₅₅ and pertussis to induce EAE. Then NECA or PBS was administered every other day on day 3, day 5, day 7 and day 9. On day 10, mice were injected intravenously with 200 μl 1% Evans Blue dye (2 μg total dye injected). Six hours after administration of Evans Blue, mice were anesthetized with a ketamine/xylazine mix and perfused via the left ventricle with ice cold PBS. Brains were harvested and homogenized in n,n-dimethylformamide (DMF) at 5 μl/mg (v:w). Tissue was incubated for 72 hours at room temperature in DMF to extract the dye. Following extraction, the tissue/solvent mixture was centrifuged at 500×g for 30 minutes and 100 μl of supernatant was read on a BioTex spectrophotometer at 620 nm. Data is expressed as pg Evans Blue/ml DMF and is shown in FIG. 17.

This experiment demonstrates that treatment of mice with the general adenosine receptor agonist NECA induces migration of Evans Blue dye into the CNS in mice immunized for EAE. This suggests that the blood brain barrier in the EAE model can be modulated through activation of the adenosine receptors. WT EAE mice treated with NECA show an increase in BBB permeability over PBS control EAE mice.

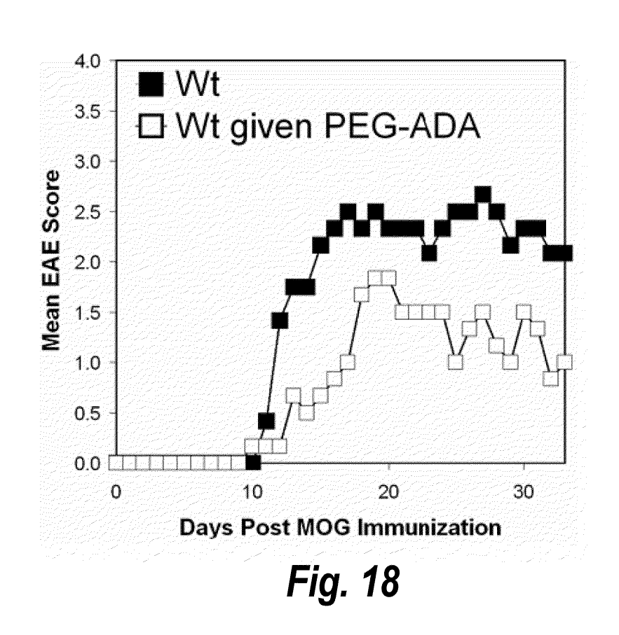

FIG. 18 shows the results in graphical form of an addition experiment that demonstrate PEGylated adenosine deaminase (“PEG-ADA”) treatment inhibits the development of EAE in wild-type mice. EAE was induced, disease activity was monitored daily, and mean EAE score was calculated in wild-type mice given either control PBS vehicle alone or 15 units/kg body weight of PEG-ADA i.p. every 4 days. Closed squares represent wild-type mice given PBS vehicle (n=3); open squares respresent wild-type mice given PEG-ADA (n=3). These results demonstrate that adenosine deaminase treatment and adenosine receptor blockade protect wild type mice against EAE induction.

### Example 22

### Mouse and Rat Models

C57BL/6 mice from Jackson Laboratories were used as wild types. All mice used were aged 7-9 weeks and weighed between 20-25 g. All rats were aged 8 weeks and weighed 200-220 g. Mice and rats were bred and housed under specific pathogen-free conditions.

### Example 23

### Administration of Drugs and Dextrans

The adenosine receptor agonists NECA, CCPA and CGS 21860 were purchased from Tocris. Each was dissolved in DMSO then diluted in PBS to the desired concentration; in most cases final DMSO concentrations were <0.5% (vol/vol). For vehicle controls, DMSO was diluted in PBS to the same concentration. Dehydrated dextrans labeled with either FITC or Texas Red were purchased from Invitrogen and re-suspended in PBS to 10 mg ml⁻¹. All experiments involving dextran injection used 0.5 mg dextran in PBS. In experiments where drug and dextran were injected concomitantly, 0.5 mg of dextran was mixed with the drug to the desired concentration in a final volume of 200 μl. All injections were retro-orbital i.v. with a 27-gauge needle. In the SCH 58261 experiment, groups were administered vehicle or SCH 58261 for 4 d. Vehicle/drug and dextrans were injected on day 5 and tissues were collected 3 h after vehicle/drug administration. In Lexiscan experiments, Lexiscan was administered i.v. with 3 injections, 5 min apart and tissues were collected at 15 min unless otherwise indicated.

### Example 24

### Treatment and Tissue Collection

In dose-response experiments and experiments with the A₁ AR and A_{2A} AR knock-out mice, drugs and dextrans were injected concomitantly. After 3 h, the mice were anesthetized with ketamine/xylazine and subjected to a nose cone containing isoflurane. They were perfused with 25-50 ml ice-cold PBS through the left ventricle of the heart then decapitated. Their brains were removed, weighed and frozen for later analysis.

### Example 25

### Fluorimetric Analysis of Dextrans in Brains

Ice-cold 50 mM Tris-Cl (pH 7.6) was added to frozen brains (100 μl per 100 mg brain) and were to thawed on ice. They were homogenized manually with ˜45 strokes of a dounce homogenizer in plastic 1.5 ml microfuge tubes then spun at 16.1 g in a microfuge for 30 min at room temperature (rt). The supernatants were transferred to new tubes and an equal volume absolute methanol was added. The samples were spun again at 16.1 g for 30 min at rt. Supernatant (200 μl) was transferred to a Corning costar 96 well black polystyrene assay plate (clear bottom). Additionally, a series of standards containing 0.001-10 μg ml⁻¹ dextran in 50% Tris-Cl/50% absolute methanol (vol/vol) was added to each plate. Absolute concentrations of dextrans were derived from these standard curves. Fluorimetric analysis was performed on a BioTek Synergy 4. FITC-dextran was detected at 488/519 (excitation/emission) and Texas Red-dextran was detected at 592/618.

### Example 26

### Cell Culture and qRT-PCR

The bEnd.3 mouse brain endothelial cell line was obtained from the ATCC and grown in ATCC formulated DMEM supplemented with 10% FBS. Using Trizol (Invitrogen) extraction, RNA was isolated from bEnd.3 cells. cDNA was synthesized using Multiscribe reverse transcriptase (Applied Biosystems). Primers (available upon request) specific for adenosine receptors and CD73 were used to determine gene expression levels and standardized to the TBP housekeeping gene levels using Kapa Sybr Fast (Kapa Biosystems) run on a BioRad CFX96 real time qPCR system. Melt curve analyses were performed to measure the specificity for each qPCR product.

### Example 27

### Injection and anti-β-Amyloid Antibodies and Immunofluorescent Microscopy

Wild type and transgenic (AD) mice were given 0.80 μg NECA i.v. After 3 h, 400 μg antibody to β-amyloid (200 μl of 2 mg ml⁻¹; clone 6E10, Covance) was administered i.v. and the mice rested for 90 min. They were then anesthetized and perfused as described above and their brains were placed in OTC and flash-frozen for later sectioning. Sagital sections (6 μm) were fixed in acetone for 10 min, then washed in PBS. Sections were blocked with casein for 20 min then incubated with 1:50 dilution of Cy5-goat anti-mouse (polyclonal, 1 mg ml⁻¹, Abcam) for 20 min then washed 3 times in PBS. Sections were then dried and mounted with Vectashield Hardset mounting media with DAPI (Vector Laboratories). Images were obtained on a Zeiss Axio Imager M1 fluorescent microscope.

### Example 28

### Analysis Confirms that NECA Increases BBB Permeability to Macromolecules

The data analyzed and the assumptions made in this study the use of a strong non-parametric statistic like the Mann-Whitney U Test. Statistical differences with the Mann-Whitney U test are indicated where P≦0.05.

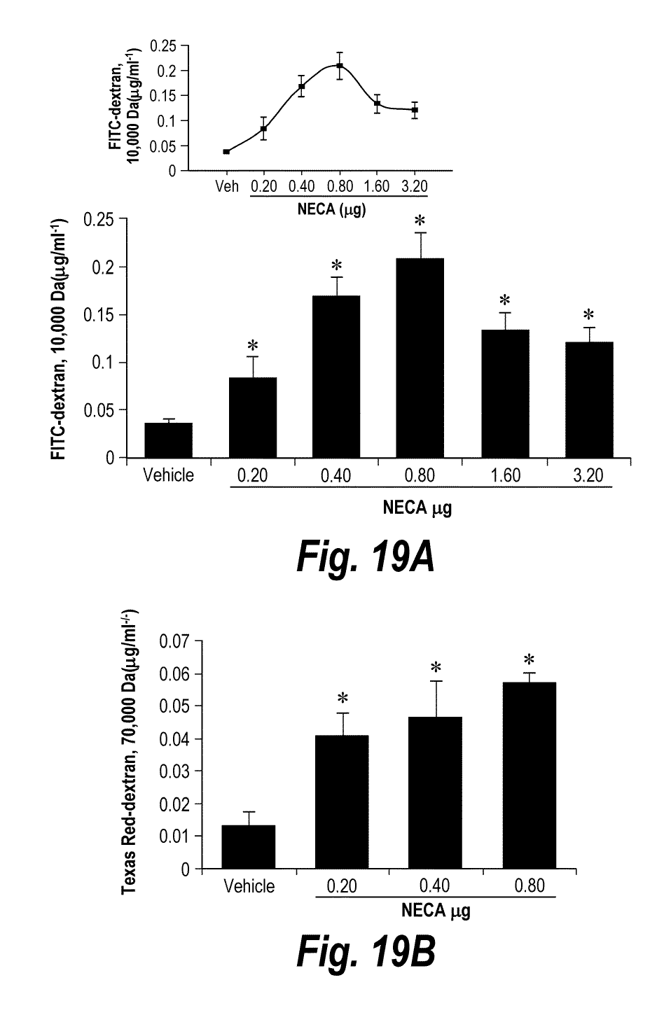

It was established that i.v. administration of NECA, which activates all ARs (A₁, A_{2A}, A_{2B}, A₃), resulted in a dose-dependent increase in extravasation of i.v.-administered fluorescently-labeled dextrans into the CNS of mice (FIG. 19). Importantly, it was observed that varying the dose of NECA resulted in a dose-dependent increase in CNS entry of both 10,000 Da dextrans (FIG. 19A) and 70,000 Da dextrans (FIG. 19B) compared to treatment with vehicle alone. Maximum entry of dextrans into the CNS was observed with 0.80 μg (100 μl of 25 μM) NECA. Higher concentrations of NECA had no additional effect or show diminished efficacy, possibly due to receptor desensitization (Ferguson et al., “Subtype-Specific Kinetics of Inhibitory Adenosine Receptor Internalization are Determined by Sensitivity to Phosphorylation by G Protein-coupled Receptor Kinases,” Mol. Pharmacol. 57:546-52 (2000), which is hereby incorporated by reference in its entirety). These results demonstrate that adenosine receptor activation increases BBB permeability.

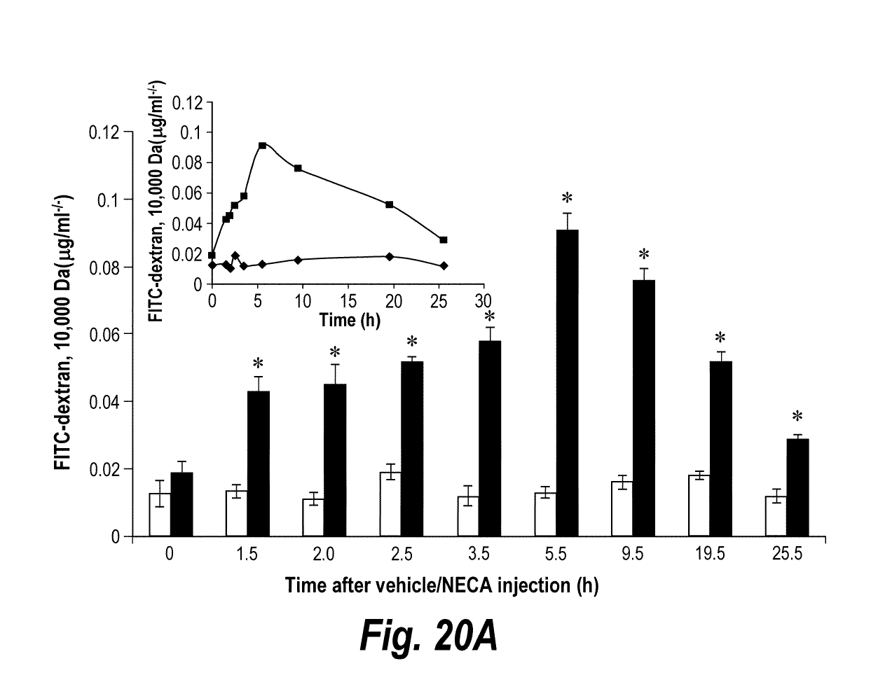

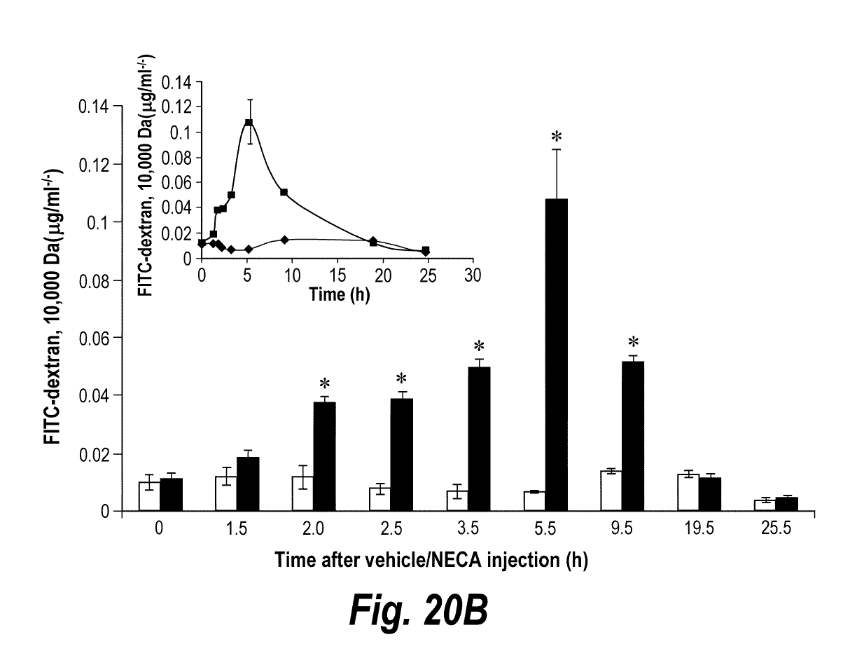

It was next determined the duration of BBB permeability after NECA administration and whether the process is reversible. Increased barrier permeability following NECA treatment is temporally discrete (FIG. 20A), with maximum entry of labeled dextran into the CNS observed between 4-6 h post-treatment. These data represent accumulation of FITC-dextran in the brain over time, since the dextran and NECA were administered at time zero (T₀). In a second experiment (FIG. 20B), dextran was administered at indicated times after NECA administration. These data represent dextran entry into the brain 90 min after dextran injection. At 8 h post-NECA treatment (9.5 h collection time), detectable levels of dextran in the brain were decreased from the maximum and by 18 h post-treatment (19.5 h collection time) the levels returned to baseline, as dextrans administered 18 h after NECA treatment were not detectable in the brain at significant levels (FIG. 20B). These results demonstrate that i.v. NECA administration results in a temporally discrete period of increased barrier permeability that returns to baseline.

### Example 29

### A₁ and A_{2A} ARs control BBB permeability

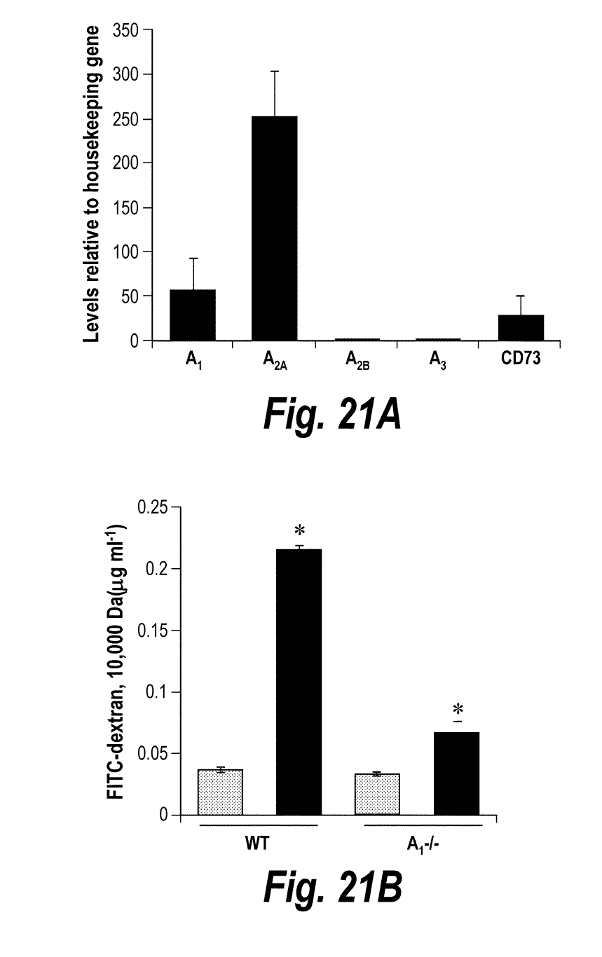

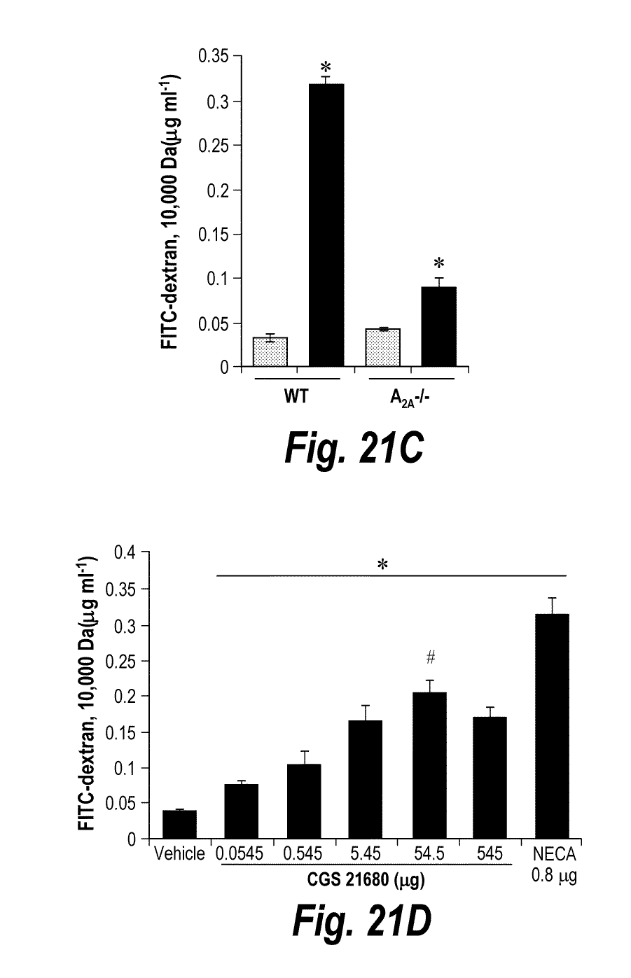

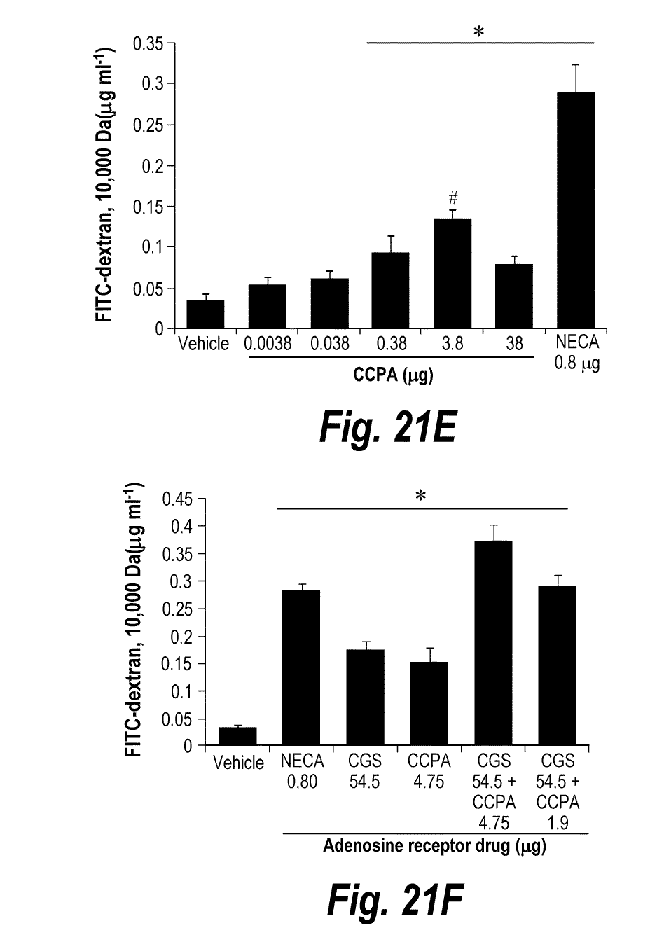

Four AR subtypes are expressed in mammals: A₁, A_{2A}, A_{2B}, and A₃ (Sebastiao et al., “Adenosine Receptors and the Central Nervous System,” Handb. Exp. Pharmacol. 471-534 (2009), which is hereby incorporated by reference in its entirety). To determine which ARs might function in barrier permeability, the levels of mRNA expression of each receptor subtype was examined in mouse brain endothelial cells. Expression of A₁ and A_{2A} receptors, but not A_{2B} or A₃ receptors, was detected in this cell line (FIG. 21A). Additionally, expression of CD73 and CD39, the two ecto-enzymes required for the catalysis of extracellular adenosine from ATP (CD39 data not shown), was observed on cultured mouse brain endothelial cells.

To investigate the functional contribution of A₁ and A_{2A} receptors in AR-mediated changes in BBB permeability, this effect was studied in mice lacking these receptors. Importantly, there were no significant differences in the basal levels of BBB permeability to 10,000 Da dextrans between WT, A₁^{-/-} and A_{2A}^{-/-} mice (FIGS. 21B and 21C). Following i.v. administration of NECA, both A₁^{-/-} and A_{2A}^{-/-} mice showed significantly lower levels of i.v.-administered dextrans in their brains compared to wild type mice (FIGS. 21B and 21C). These data suggest that modulation of barrier permeability is, at least in part, mediated by these two AR subtypes. To confirm these results, the specific A₁ agonist 2-chloro-N⁶-cyclopentyladenosine (CCPA) and the specific A_{2A} agonist 4-[2-[[6-Amino-9-(N-ethyl-b-D-ribofuranuronamidosyl)-9H-purin-2-yl]amino]ethyl]benzenepropanoic acid (CGS 21680) were administered to wild type mice. Both CGS 21680 (FIG. 21D) and CCPA (FIG. 21E) treatment resulted in increased dextran entry into the CNS and while this increase is substantial compared to vehicle treatment it was significantly lower than that observed after NECA administration. However, when used in combination, CCPA and CGS 21680 recapitulated the effect of increased dextran entry into the CNS that was observed with NECA treatment (FIG. 21F). These results confirmed that modulation of adenosine receptors facilitates entry of molecules into the CNS.

### Example 30

### The Specific A2A Agonist Lexiscan increases BBB Permeability

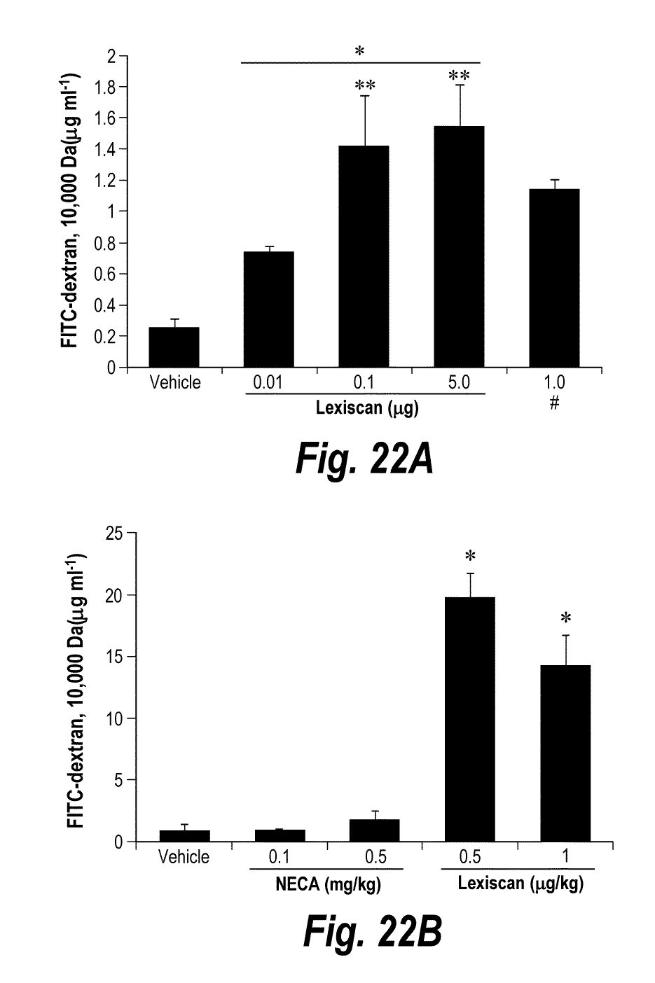

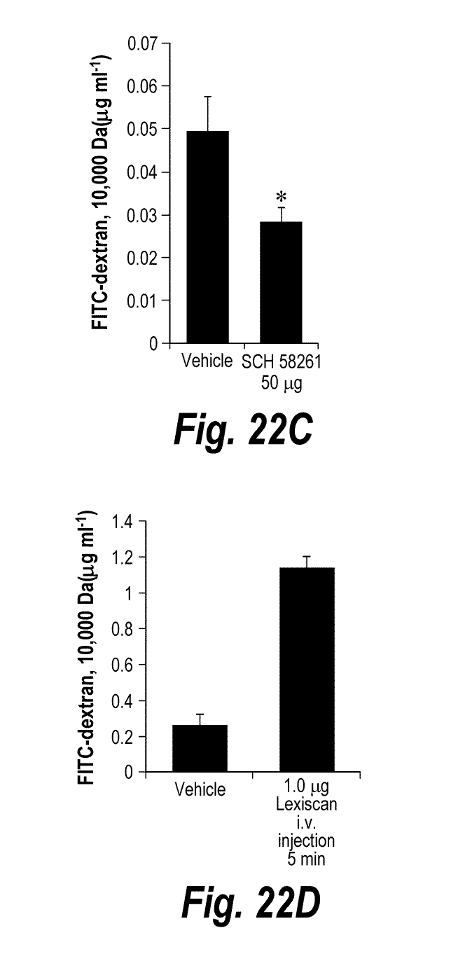

To expand the possible therapeutic use of AR agonism to facilitate CNS entry of i.v.-administered compounds, a commercially-available, FDA-approved AR agonist was tested in the experimental paradigm. The specific A_{2A} AR agonist Lexiscan, which has been successfully used in myocardial perfusion imaging (Iskandrian et al., “Adenosine Versus Regadenoson Comparative Evaluation in Myocardial Perfusion Imaging: Results of the ADVANCE Phase 3 Multicenter International Trial,” J. Nucl. Cardiol. 14:645-58 (2007), which is hereby incorporated by reference in its entirety), did indeed increase BBB permeability to 10,000 Da dextrans after i.v. administration (FIG. 22A) in mice. Interestingly, FITC-dextran was detectable in the brain after 5 min following a single Lexiscan injection. Additionally, i.v. administration of Lexiscan also increased BBB permeability in rats (FIG. 22B). The magnitude of increased BBB permeability after Lexiscan administration was much greater than the magnitude of increased permeability after NECA administration. These results demonstrate that in addition to the broad AR agonist, NECA, and the specific A₁ and A_{2A} AR agonists, CCPA and CGS 21680, used in this study, the FDA-approved A_{2A} agonist Lexiscan can increase BBB permeability to macromolecules.

### Example 31

### A_{2A} Antagonism Decreases BBB Permeability

It was further hypothesized that if agonism of A₁ and A_{2A} receptors increases barrier permeability, then AR antagonism might decrease barrier permeability and prevent molecules from entering the CNS. It was previously observed that in WT mice, blockade of the A_{2A} adenosine receptor inhibited leukocyte migration into the CNS (Mills et al., “CD73 is Required for Efficient Entry of Lymphocytes Into the Central Nervous System During Experimental Autoimmune Encephalomyelitis,” Proc Natl Acad Sci USA 105: 9325-30 (2008), which is hereby incorporated by reference in its entirety). This hypothesis was tested with a specific A_{2A} AR antagonist. Intraperitoneal administration of the A_{2A} AR antagonist 2-(2-Furanyl)-7-(2-phenylethyl)-7H-pyrazolo[4,3-e][1,2,4]triazolo[1,5-c]pyrimidin-5-amine (SCH 58261) resulted in significantly decreased entry of 10,000 Da FITC-dextran into WT mice brains (FIG. 22C). This data supports that blocking AR signaling tightens or closes the BBB.

### Example 32

### Antibodies to β-amyloid enter the brain after NECA administration

The most challenging therapeutic agents to get across the BBB are macromolecules such as antibodies, due to their enormous size (˜150 kDa). It was asked whether adenosine receptor modulation with NECA can facilitate the entry of antibodies into the CNS. To test this, a double [amyloid precursor protein (APP)/presenilin (PSEN)] transgenic mouse model of AD [strain B6.Cg-Tg(APPswe,PSEN1dE9)85Dbo/J] was used. These mice accumulate similar β-amyloid (Aβ) plaques that are a hallmark of AD (Mineur et al., “Genetic Mouse Models of Alzheimer's Disease,” Neural. Plast. 12:299-310 (2005), which is hereby incorporated by reference in its entirety).

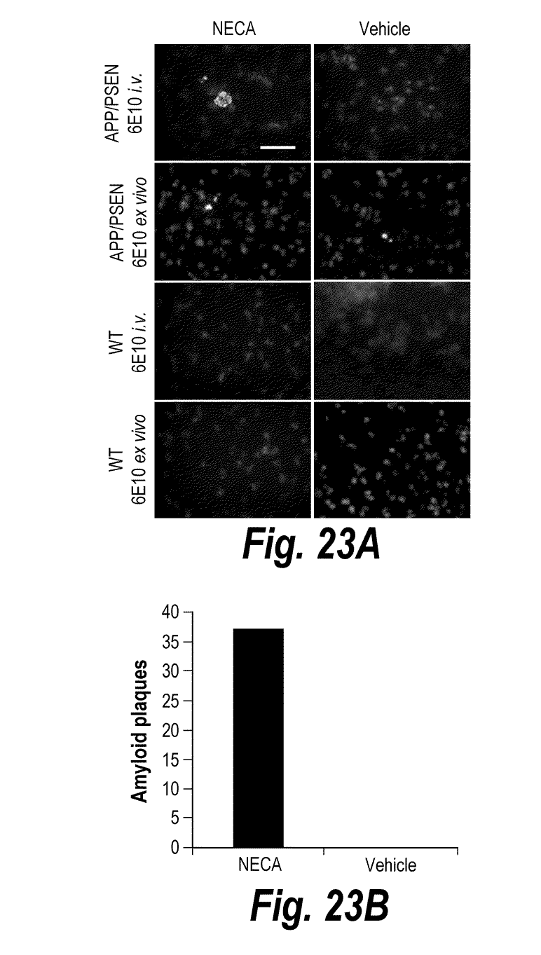

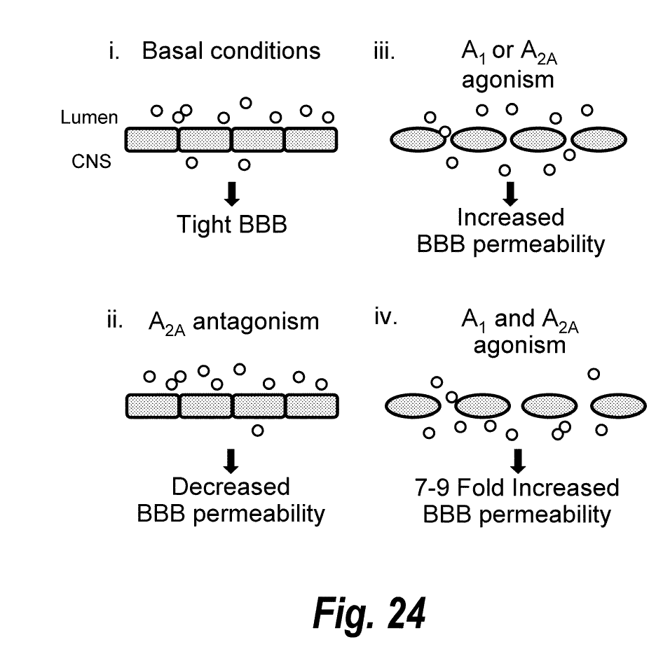

The monoclonal antibody 6E10 (Covance) has been shown to significantly reduce Aβ plaque burden in a mouse model of AD when administered by intracerebroventricular injection (Thakker et al., “Intracerebroventricular Amyloid-beta Antibodies Reduce Cerebral Amyloid Angiopathy and Associated Micro-hemorrhages in Aged Tg2576 Mice,” Proc. Natl. Acad. Sci. USA 106:4501-6 (2009), which is hereby incorporated by reference in its entirety). Three hours after i.v. NECA administration, the 6E10 antibody i.v. was administered. After 90 min, brains were collected, sectioned and stained with a secondary Cy5-labeled antibody. Binding of 6E10 antibody to Aβ plaques was observed throughout the brains of NECA-treated mice, with a concentration of Aβ plaques in the hippocampal region (FIG. 23A). No binding of 6E10 antibody was observed in mice treated with vehicle alone (FIGS. 23A and 23B). These results demonstrate that antibody to β-amyloid administered i.v. can cross the BBB after AR agonism.

### Example 32

### Adenosine Signaling Induces Actomyosin Stress Fiber Formation in Endothelial Cells

Since actomyosin stress fibers are necessary for inducing contraction in cell shape (Hotulainen et al., “Stress Fibers are Generated by Two Distinct Actin Assembly Mechanisms in Motile Cells,” J. Cell. Biol. 173:383-94 (2006), which is hereby incorporated by reference in its entirety), it was hypothesized that adenosine receptor signaling results in actin stress fiber induction.

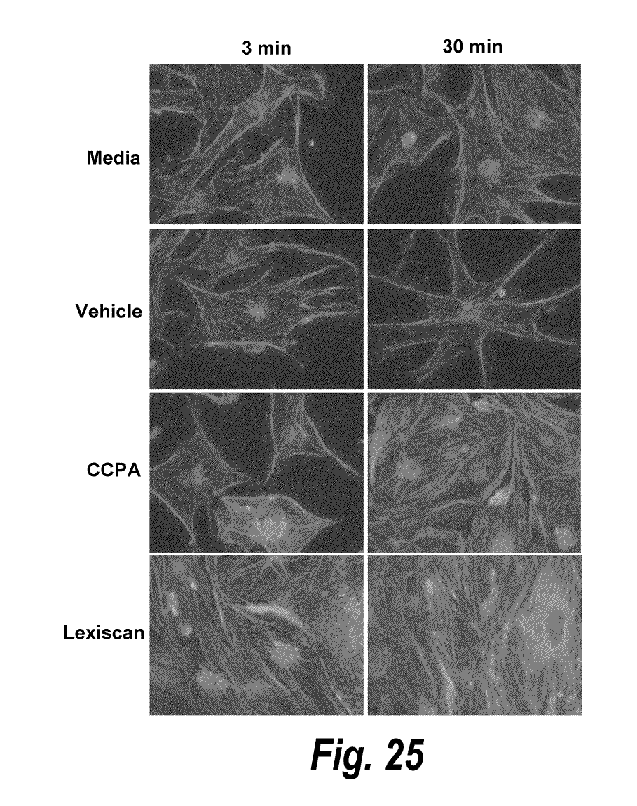

To test this brain endothelial cells were treated with either CCPA (to agonize A₁ adenosine receptors) or Lexiscan (to agonize the A_{2A} adenosine receptor). The induction of actin stress fibers was observed upon A₁ and A_{zA} agonist treatment as compared to treatment with vehicle alone, as shown in FIG. 25.

Although embodiments are depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

We claim:

1. A method for delivering a small molecule therapeutic agent to the brain of a subject, comprising administering to said subject: (a) an agent which activates both of A₁ and A₂ₐ adenosine receptors; and (b) a small molecule therapeutic agent.

2. The method according to claim 1, wherein the agent which activates both A₁ and A₂ₐ adenosine receptors is an agonist of both A₁ and A₂ₐ adenosine receptors.

3. The method according to claim 2, wherein the agonist of both A₁ and A₂ₐ receptors is selected from adenosine, NECA, AMP-579, metrifudil, and 8-butylaminoadenosine (BAA).

4. The method according to claim 1, wherein the activation of both A₁ and A₂ₐ receptors is synergistic with respect to blood brain barrier permeability.

5. The method according to claim 1, wherein the activation of both A₁ and A₂ₐ receptors is additive with respect to blood brain barrier permeability.

6. A method for delivering a small molecule therapeutic agent to the brain of a subject, comprising administering to said subject: (a) an A₁ adenosine receptor agonist and an A₂ₐ adenosine receptor agonist; and (b) small molecule therapeutic agent.

7. The method according to claim 6, wherein the A₁ adenosine receptor agonist and A₂ₐ adenosine receptor agonist are A₁-selective and A₂ₐ-selective adenosine receptor agonists.

8. The method according to claim 6, wherein the A₁ adenosine receptor agonist and A₂ₐ adenosine receptor agonist are formulated in a single unit dosage form.

9. The method according to claim 6, wherein the A₁ adenosine receptor agonist and A₂ₐ adenosine receptor agonist are administered simultaneously.

10. The method according to claim 6, wherein the A₁ adenosine receptor agonist and A₂ₐ adenosine receptor agonist are administered sequentially.

11. The method according to claim 7, wherein the A₁-selective adenosine receptor agonist is selected from CCPA, 8-cyclopentyl-1,3-dipropylxanthine, R-phenylisopropyl adenosine and N6-cyclopentyladenosine.

12. The method according to claim 7, wherein the A₂ₐ-selective adenosine receptor agonist is selected from regadenoson (Lexiscan®), apadenoson, binodenoson, CGS 21680 (4-[2-[[6-Amino-9-(N-ethyl-b-D-ribofuranuronamidosyl)-9H-purin-2yl]amino]ethyl]benzenepropanoic acid), ATL-146e, YT-146 (2-(1-octynyl)adenosine) and DPMA (N⁶-(2-(3,5-dimethoxyphenyl)-2-(2-methylphenyl)ethyl)adenosine).

13. The method according to claim 1, wherein the small molecule therapeutic agent is selected from acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenyloin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methotrexate, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, paroxetine, pergolide, periciazine, perphenazine, phenacemide, phenelzine, phenobarbitol, phenturide, phenyloin, pimozide, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine or zuclopenthixol.

14. The method according to claim 2, wherein the A₁/A₂ₐ adenosine receptor agonist is administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the small molecule therapeutic agent.

15. A method for administering a therapeutic agent across the choroid plexus of a subject comprising administering to the subject (a) the therapeutic agent; and (b) an agent for increasing the permeability of the choroid plexus in a subject.

16. The method according to claim 15, wherein the therapeutic agent is a small molecule or a polynucleotide.

17. The method according to claim 16, wherein the small molecule is selected from acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenyloin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methotrexate, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, paroxetine, pergolide, periciazine, perphenazine, phenacemide, phenelzine, phenobarbitol, phenturide, phenyloin, pimozide, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine or zuclopenthixol.

18. The method according to claim 16, wherein the polynucleotide is selected from plasmid DNA (pDNA), short-interfering RNA (siRNA), short-hairpin RNA (shRNA), microRNA (miRNA), messenger RNA (mRNA) and antisense RNA (aRNA).

19. The method according to claim 16, wherein the agent for increasing the permeability of the choroid plexus is an A₁ adenosine receptor antagonist.

20. The method according to claim 19, wherein the A₁ adenosine receptor antagonist is selected from caffeine, theophylline, cyclopentyltheophylline (CPT), 8-cyclopentyl-1,3-dimethylxanthine, 8-cyclopentyl-1,3-dipropylxanthine (DPCPX), 8-phenyl-1,3-dipropylxanthine, bamifylline, BG-9719, BG-9928, FK-453, FK-838, rolofylline (KW-3902), N-0861, CGS-15943 (9-chloro-2-(2-furanyl)-[1,2,4]-triazolo[1,5-c]-quinazolin-5-amine, and PSB 36 (1-butyl-8-(hexahydro-2,5-methanopentalen-3a-(1H)-yl-3,7-dihydro-3-(3-hydroxypropyl)-1H-purine-2,6-dione).

21. The method according to claim 19, wherein the A₁ adenosine receptor antagonist is administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent.

22. A method for treating a CNS disease, disorder, or condition in a subject, comprising administering to said subject (a) at least one agent which activates both of A₁ and A₂ₐ adenosine receptors; and (b) a therapeutic agent.

23. The method according to claim 22, wherein the therapeutic agent is a small molecule or a polynucleotide.

24. The method according to claim 23, wherein the CNS disease, disorder, or condition is selected from a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome.

25. The method according to claim 23, wherein the small molecule is selected from acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenyloin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methotrexate, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, paroxetine, pergolide, periciazine, perphenazine, phenacemide, phenelzine, phenobarbitol, phenturide, phenyloin, pimozide, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine or zuclopenthixol.

26. The method according to claim 23, wherein the polynucleotide is selected from plasmid DNA (pDNA), short-interfering RNA (siRNA), short-hairpin RNA (shRNA), microRNA (miRNA), messenger RNA (mRNA) and antisense RNA (aRNA).

27. The method according to claim 22, wherein the activation of both A₁ and A₂ₐ receptors is synergistic with respect to blood brain barrier permeability.

28. The method according to claim 22, wherein the activation of both A₁ and A₂ₐ receptors is additive with respect to blood brain barrier permeability.

29. A method for treating a CNS disease, disorder, or condition in a subject, comprising administering to said subject (a) an A₁ adenosine receptor agonist and an A₂ₐ adenosine receptor agonist; and (b) a small molecule therapeutic agent.

30. The method according to claim 29, wherein the A₁ adenosine receptor agonist and A₂ₐ adenosine receptor agonist are A₁-selective and A₂-selective adenosine receptor agonists.

31. The method according to claim 29, wherein the A₁ adenosine receptor agonist and A₂ₐ adenosine receptor agonist are formulated in a single unit dosage form.

32. The method according to claim 29, wherein the A₁ adenosine receptor agonist and A₂ₐ adenosine receptor agonist are administered simultaneously.

33. The method according to claim 29, wherein the A₁ adenosine receptor agonist and A₂ₐ adenosine receptor agonist are administered sequentially.

34. The method according to claim 29, wherein the A₁ adenosine receptor agonist and A₂ₐ adenosine receptor agonist are administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the small molecule therapeutic agent.

35. A method for delivering a therapeutic agent to the brain of a subject, comprising administering to said subject: (a) an A₂ₐ adenosine receptor agonist; and (b) the therapeutic agent.

36. The method according to claim 35, wherein the A₂ₐ adenosine receptor agonist is selected from regadenoson (Lexiscan®), apadenoson, binodenoson, CGS 21680 (4-[2-[[6-Amino-9-(N-ethyl-b-D-ribofuranuronamidosyl)-9H-purin-2yl]amino]ethyl]benzenepropanoic acid), ATL-146e, YT-146 (2-(1-octynyl)adenosine) and DPMA (N⁶-(2-(3,5-dimethoxyphenyl)-2-(2-methylphenyl)ethyl)adenosine).

37. The method according to claim 35, wherein the therapeutic agent is a small molecule or a polynucleotide.

38. The method according to claim 37, wherein the small molecule is selected from acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenyloin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methotrexate, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, paroxetine, pergolide, periciazine, perphenazine, phenacemide, phenelzine, phenobarbitol, phenturide, phenyloin, pimozide, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine or zuclopenthixol.

39. The method according to claim 37, wherein the polynucleotide is selected from plasmid DNA (pDNA), short-interfering RNA (siRNA), short-hairpin RNA (shRNA), microRNA (miRNA), messenger RNA (mRNA) and antisense RNA (aRNA).

40. The method according to claim 35, wherein the A₂ₐ adenosine receptor agonist is administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent.

41. A method for treating a CNS disease, disorder, or condition in a subject, comprising administering to said subject (a) an A₂ₐ adenosine receptor agonist; and (b) a therapeutic agent.

42. The method according to claim 41, wherein the therapeutic agent is a small molecule or a polynucleotide.

43. The method according to claim 42, wherein the CNS disease, disorder, or condition is selected from a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome.

44. The method according to claim 42, wherein the small molecule is selected from acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenyloin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methotrexate, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, paroxetine, pergolide, periciazine, perphenazine, phenacemide, phenelzine, phenobarbitol, phenturide, phenyloin, pimozide, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine or zuclopenthixol.

45. The method according to claim 42, wherein the polynucleotide is selected from plasmid DNA (pDNA), short-interfering RNA (siRNA), short-hairpin RNA (shRNA), microRNA (miRNA), messenger RNA (mRNA) and antisense RNA (aRNA).
